(19) United States
(12) Patent Application Publication
(10) Pub. No.: US 2014/0057838 A1
(43) Pub. Date: Feb. 27, 2014

(54) OSTEOBLAST-EXPRESSED LIPOCALIN 2 REGULATES GLUCOSE METABOLISM

(71) Applicant: Stavroula Kousteni, Glen Ridge, NJ (US)

(72) Inventor: Stavroula Kousteni, Glen Ridge, NJ (US)

(73) Assignee: THE TRUSTEES OF COLUMBIA UNIVERSITY IN THE CITY OF NEW YORK, NEW YORK, NY (US)

(21) Appl. No.: 14/111,324

(22) PCT Filed: Apr. 11, 2012
(86) PCT No.: PCT/US12/33164

§ 371 Date: Nov. 12, 2013

Related U.S. Application Data
(60) Provisional application No. 61/474,184, filed on Apr. 11, 2011.

(51) Int. Cl. A61K 38/17; A61K 45/06

(52) U.S. Cl. CPC A61K 38/1709; A61K 45/06
USPC 514/4.9; 514/6.9; 514/7.3; 514/4.8; 514/7.4; 514/16.5; 435/375; 435/366

(57) ABSTRACT

Diseases including diabetes, metabolic syndrome, and obesity or obesity-related diseases are due to impairment in glucose metabolism. The skeleton has been shown to regulate energy metabolism and play a role in glucose metabolism. The present invention relates to methods for treating or preventing diseases such as diabetes, metabolic syndrome, and obesity or obesity-related by administering a therapeutically effective amount of osteoblast-expressed Lcn-2 or a biologically active fragment.

### CROSS-REFERENCE TO RELATED APPLICATIONS

This application claims benefit of U.S. Provisional Application Ser. No. 60/474,184, entitled “Osteoblast-Expressed Lipocalin 2 Regulates Glucose Metabolism,” filed Apr. 11, 2011, the entire contents of which are hereby incorporated by reference as if fully set forth herein, under 35 U.S.C. §119(e).

### STATEMENT OF GOVERNMENTAL INTEREST

This invention was made with Government support under Contract No. AR055931 awarded by the National Institutes of Health. The Government has certain rights in the invention.

### BACKGROUND OF THE INVENTION

Diseases associated with glucose metabolism impairment such as diabetes, metabolic syndrome, and obesity, are severe conditions connected with high mortality. Among United States residents aged 65 years and older, 10.9 million, or 26.9%, had diabetes in 2010.¹ About 215,000 people younger than 20 years had diabetes (type 1 or type 2) in the United States in 2010.¹ Nearly 1.9 million people aged 20 years or older were newly diagnosed with diabetes in 2010 in the United States.¹ Diabetes is the leading cause of kidney failure, nontraumatic lower-limb amputations, and new cases of blindness among adults in the United States.¹ As a major cause of heart disease and stroke, diabetes is the seventh leading cause of death in the United States.¹ National obesity trends are less encouraging. More than one-third of United States adults (35.7%) are obese.² Approximately 17% (or 12.5 million) of children and adolescents aged 2-19 years are obese.

During the past 20 years, there has been a dramatic increase in obesity in the United States and rates remain high. In 2010, no state had a prevalence of obesity less than 20%.² Not only do both diabetes and obesity threaten a significant portion of the United States population, this health crisis is a considerable financial burden as well. There is no shortage of research in the United States in an effort to combat diabetes, metabolic syndrome, obesity, and obesity-related diseases.

Recent evidence suggests that the skeleton plays a role in energy metabolism. Through the osteoblast-secreted hormone osteocalcin, the skeleton regulates energy metabolism and hematopoiesis and is associated with insulin secretion, insulin sensitivity, and energy expenditure. Skeletal functions have been shown to expand beyond regulation of bone mass homeostasis to affect whole body physiology. Therefore, there is a great need to have a greater understanding of how the body regulates glucose as well as a need for new methods and compositions to treat diseases associated with impairment of glucose metabolism such as diabetes, metabolic syndrome, and obesity.

### SUMMARY OF THE INVENTION

It has been discovered that osteoblast-expressed lipocalin-2 (Lcn-2) is a potent regulator of myogenesis and muscle sensitivity and plays a role in glucose metabolism. Therefore, administration of Lcn-2 or a biologically active fragment or variant is effective in treating disorders or diseases where glucose metabolism is impaired. A first set of embodiments of the invention is directed to methods for identifying a subject having a disorder such as type 1 diabetes or type 2 diabetes, metabolic syndrome, obesity or obesity-related disease, and administering to the subject a therapeutically effective amount of Lcn-2 or a biologically active fragment or variant thereby treating or preventing the disorder. In certain embodiments, the subject is a human. Where Lcn-2 or a biologically active fragment or variant is administered, it is administered in an amount effective to produce an effect selected from the group comprising an increase in pancreatic β-cell proliferation, an increase in insulin expression, an increase in insulin sensitivity, an increase in glucose tolerance, a decrease in weight gain, a decrease in fat mass, an increase in weight loss, an increase in bone mass, an increase in adiponectin serum levels, and a decrease in serum resistin levels. Lcn-2 or a biologically active fragment or variant can also be administered in combination with an agent known to treat the disorder. Other embodiments include therapeutically effective amounts of Lcn-2 or a biologically active fragment or variant. An indicative does range in humans would be from about 4 to 60 micrograms/kg (4.05 to 40.5 micrograms/kg for adults and 6 to 60 micrograms/kg for children).

In other embodiments of the invention, methods are directed to administering Lcn-2 or a biologically active fragment or variant to a subject in an amount that causes an effect selected from the group consisting of a significant increase in pancreatic β-cell proliferation, insulin expression, insulin sensitivity, glucose tolerance, a decrease weight gain, a decrease in fat mass, an increase weight loss, an increase in bone mass, an increase in adiponectin serum levels, and a decrease in serum resistin levels. The subject can be a human.

Where disorders of glucose metabolism such as diabetes, metabolic syndrome, and obesity, or obesity-related diseases are being treated with a therapeutically effective amount of Lcn-2 or a biologically active fragment or variant in a subject, an embodiment of the method further comprises co-administration of one or more agents (e.g., anti-diabetic drugs) or drugs to treat complications or conditions associated with diabetes either in the same or in separate pharmaceutical compositions and kits, either on the same day or on different days. Certain embodiments are directed to formulations of Lcn-2 alone or with other active agents (such as anti-diabetic drugs) that have a therapeutic use to treat or prevent one or more of the disorders described. Therapeutically effective amounts of Lcn-2 or a biologically active fragments or variants may be administered with resistin, adiponectin, and therapeutic oligonucleotides that reduce the expression or biological activity of FoxO1. In certain embodiments of the invention, the therapeutic amount produces an effect selected from the group consisting of a significant increase in pancreatic β-cell proliferation, insulin expression, insulin sensitivity, glucose tolerance, a decrease weight gain, a decrease in fat mass, an increase weight loss, an increase in bone mass, an increase in adiponectin serum levels, and a decrease in serum resistin levels. The subject can be a human.

Another embodiment is directed to a method comprising identifying a subject having or at risk of developing type 1 or type 2 diabetes, metabolic syndrome, obesity, or obesity-related diseases and administering to the subject a therapeutically effective amount of an agent that reduces Forkhead Box Protein O1 (FoxO1) expression or FoxO1 activity (either alone or together with Lcn-2), preferably in osteoblasts, in an amount that increases serum Lcn-2 levels. In certain embodiments, the agent is an isolated nucleic acid that is selected from the group consisting of cDNA, antisense DNA, antisense RNA, micro RNA (miRNA), ribozymes, and small interfering RNA (siRNA). The nucleic acid is sufficiently complementary to the gene or mRNA encoding FoxO1 to permit specific hybridization to the gene or mRNA, wherein the hybridization prevents or reduces expression of FoxO1 in osteoblasts. The miRNA can be selected from the group consisting of miR-182, miR-96, miR-183, and miR-135b.

Other embodiments are directed to methods comprising identifying a subject having or at risk of developing a disorder of the bone in a subject by administering therapeutically effective amounts of Lcn-2 or biologically active fragments or variants, or an inhibitory oligonucleotide that reduces Fox01 expression or FoxO1 activity. Such methods are effective in treating or preventing the bone disorder. The bone disorder includes osteoporosis, osteopenia, osteomyelitis, and osteoarthritis. Bone disorders may be the result of bone loss due to any disease or treatment for disease causing bone loss, including, but not limited to treatment for cancer.

In certain other embodiments, methods are directed to identifying a subject having or at risk of developing a muscle disorder and administering a therapeutically effective amount of Lcn-2 or a biologically active fragment or variant and/or or an inhibitory oligonucleotide that reduces FoxO1 expression or activity to the subject in an amount that increases or maintain myogenesis and thereby treats or prevents the muscle disorder. The muscle disorder is selected from the group consisting of muscle atrophy, muscular dystrophy, fibromyalgia, myositis, polymyositis, myopathy, rhabdomyolysis, inflammatory muscle disease, MCAD, and other fatty acid oxidation disorders and caritine/acylcarnitine translocase deficiency or CACT.

Other embodiments of the invention include methods for increasing beta-cell area or beta-cell numbers in pancreatic cells in a subject in need, by contacting pancreatic beta cells in vivo or in vitro with a therapeutically effective amount of an agent. Preferably, the agent is Lcn-2 or a biologically active fragment or variant. The beta cells are human cells.

These and other features, aspects, and advantages of the present invention will become better understood with regard to the following description, appended claims, and accompanying figures.

### BRIEF DESCRIPTION OF THE DRAWINGS

The present invention is illustrated by way of example, and not by way of limitation, in the figures and in which like reference numerals refer to similar elements and in which:

FIG. 1 (FIG. 1): Lcn-2 expression is upregulated in the femur of 1-month old female FoxO1_{osb}−/− mice as measured by the level of relative mRNA expression.

FIG. 2 (FIG. 2): Lcn-2 serum levels increase in FoxO1_{osb}−/− mice of different genetic backgrounds, i.e. C57BL6 and Agouti.

FIG. 3 (FIG. 3): Lcn-2 is preferentially expressed in bone (the femur) as compared to white adipose tissue as measured by relative Lcn-2 expression levels.

FIG. 4 (FIG. 4): Lcn-2 is preferentially expressed in osteoblasts as compared to adipocytes as measured by Lcn-2 relative mRNA expression levels.

FIG. 5 (FIG. 5): Lcn-2 is preferentially expressed in bone marrow derived stromal cells as compared to adipocytes as measured by Lcn-2 relative mRNA expression levels.

FIG. 6 (FIG. 6): Recombinant Lcn-2 increases Ins1 (A) and Ins2 (B) expression in INS-1 cells.

FIG. 7 (FIG. 7): Recombinant Lcn-2 increases expression of markers of cell proliferation in INS-1 cells: (A) cyclin D2 and (B) cdk-4.

FIG. 8 (FIG. 8): Recombinant Lcn-2 increases the expression of PPARα (a key regulator of fatty acid oxidation in skeletal muscle) in C2C12 myocytes.

FIG. 9 (FIG. 9): Recombinant Lcn-2 increases the expression of PGC-1α (PGC-α is a coactivator of PPARα, an insulin target, highly expressed in metabolically active tissues including brown fat, skeletal muscle and heart and involved in mitochondrial myogenesis and increased mitochondrial respiration) in C2C12 myocytes.

FIG. 10 (FIG. 10): Recombinant Lcn-2 increases the expression of Nrf-1 (Pgc-1 target) in C2C12 myocytes.

FIG. 11 (FIG. 11): Recombinant Lcn-2 increases the expression of Mcad (Pgc-1 target) in C2C12 myocytes.

FIG. 12 (FIG. 12): Lcn-2 is an osteoblast-specific secreted molecule. Gene expression analysis by quantitative real time PCR in a variety of mouse tissues indicated that Lcn-2 is specifically expressed by bone cells, with a 10-fold lower expression by the testis.

FIG. 13 (FIG. 13): Recombinant Lcn-2 increases the expression of Mrf-4 (muscle regulatory factor-4 involved in regulation of myogenesis) in C2C12 myocytes.

FIG. 14 (FIG. 14): Recombinant Lcn-2 increases the expression of the insulin-sensitizing hormone Adiponectin in 3T3-L1 adipocytes.

FIG. 15 (FIG. 15): Recombinant Lcn-2 decreases the expression of Resistin in 3T3-L1 adipocytes.

FIG. 16 (FIG. 16): At 8 weeks following Lcn-2 treatment, glucose tolerance testing (GTT) indicated that mice treated with Lcn-2 had improved glucose tolerance with response reaching a peak at 150 ng/g. Blood glucose levels (mg/dL) were measured at 0, 15, 30, 60, and 120 minutes after injection.

FIG. 17 (FIG. 17): The improvement of glucose tolerance was also evident at 12 weeks following initiation of Lcn-2 treatment. Blood glucose levels (mg/dL) were measured at 15, 30, 60, and 120 minutes.

FIG. 18 (FIG. 18): Recombinant Lcn-2 treated mice demonstrated higher insulin levels after glucose challenge at every time point measured. Glucose-stimulated insulin secretion (GSIS) testing was performed at week 10 of treatment. Serum insulin levels (ng/ml) were measured 0, 10, 20, 30, and 40 minutes after glucose injection.

FIG. 19 (FIG. 19): Histological analysis of pancreatic sections at 20× magnification shows pancreas-insulin staining that in agreement with the increased levels of serum levels in Lcn-2-treated mice, these same animals showed increased β-cell area and β-cell numbers at 150 ng/g/day.

FIG. 20 (FIG. 20): Histological analysis of pancreatic sections at 40× magnification shows pancreas-insulin staining that in agreement with the increased levels of serum levels in Lcn-2-treated mice, these same animals showed increased β-cell area and β-cell numbers at 150 ng/g/day.

FIG. 21 (FIG. 21): Increase in (A) islet number (1/MM²) and (B) B cell area (%) for recombinant Len-2 treated mice.

FIG. 22 (FIG. 22): Lcn-2-treated mice demonstrated improved insulin sensitivity as examined by an insulin tolerance test (ITT) at 11 weeks as measured by % initial glucose at 0, 15, 30, 60, 90, and 120 minutes for vehicle and 150 ng/g/day.

FIG. 23 (FIG. 23): Starting at 4 weeks and throughout treatment at 8 weeks, 12, weeks, and 16 weeks, Lcn-2-treated mice demonstrated lower fat mass as compared to untreated animals. Aging mice progressively gained fat mass. Mice treated with Lcn-2 gained a lot less with a progressive increase in the difference between treated and untreated animals. Fat mass (g) measurements were performed using an MRI machine.

FIG. 24 (FIG. 24): Lcn-2-treated mice had a decreased fat mass (g) compared to untreated mice measured at 4 weeks, 8 weeks, 12 weeks, and 16 weeks.

FIG. 25 (A)-(B) (FIG. 25) (A)-(B): Lcn-2-treated mice had an increased lean mass (g) compared to the untreated mice measured at 4 weeks, 8 weeks, 12 weeks, and 16 weeks.

FIG. 26 (A)-(E) (FIG. 26) (A)-(E): Lcn-2 treatment of mice increases energy expenditure and activity; calorimetric measurements also indicated that Lcn-2 increased energy expenditure (measure by the volume of O₂ and CO₂, and by heat production) and also increased activity.

FIG. 27 (A)-(B) (FIG. 27) (A)-(B): Lcn-2 treatment of mice decreased food intake (g/24 h) measured at 50 ng/d/day, 150 ng/d/day, and 500 ng/d/day.

FIG. 28(A)-(C) (FIG. 28) (A)-(C): After 18 weeks of treatment, Lcn-2 suppresses the expression of adipogenic/lipogenic genes in white adipose tissue. Expression (relative mRNA levels) of lipolytic genes (A) Triglyceride lipase (Tgl); (B) Perillipin; and (C) Lipoprotein lipase (Lpl) measured at 50 ng/g/day, 150 ng/g/day, and 500 ng/g/day was decreased.

FIG. 29 (A)-(B) (FIG. 29) (A)-(B): Lcn-2 suppresses the expression (relative mRNA levels) of two major genes promoting adipocyte differentiation: (A) C/EBPα and (B) PPARγ in white adipose tissue measured at 50 ng/g/day, 150 ng/g/day, and 500 ng/g/day.

FIG. 30 (FIG. 30): Lcn-2 treated mice demonstrated a decrease in the expression (relative mRNA levels) of RBP-4 (Retinol binding protein 4) measured at 50 ng/g/day, 150 ng/g/day, and 500 ng/g/day in white adipose tissue. RBP-4 is an adipokine shown to promote insulin resistance and associated with obesity and diabetes in mice and humans.

FIG. 31 (A)-(E) (FIG. 31) (A)-(E): In muscle, Len-2 treatment increased the expression (relative mRNA levels) of genes for Acyl-CoA that promote myogenesis and fatty acid beta-oxidation as measured in four muscles: (A) Tibialis anterior; (B) Extensor digitorum longus; (C) soleus; (D) quadriceps; and (E) all four muscles combined measured at 50 ng/g/day, 150 ng/g/day, and 500 ng/g/day.

FIG. 32(A)-(E) (FIG. 32) (A)-(E): In muscle, Lcn-2 treatment increased the expression (relative mRNA levels) of genes for Mcad as measured in four muscles: (A) Tibialis anterior; (B) Extensor digitorum longus; (C) soleus; (D) quadriceps; and (E) all four muscles combined measured at 50 ng/d/day, 150 ng/g/day, and 500 ng/g/day.

FIG. 33 (A)-(E) (FIG. 33) (A)-(E): In muscle, Lcn-2 treatment increased the expression (relative mRNA levels) of genes for NRF-1 as measured in four muscles, (A) Tibialis anterior; (B) Extensor digitorum longus; (C) soleus; (D) quadriceps; and (E) all four muscles combined measured at 50 ng/d/day, 150 ng/g/day, and 500 ng/g/day.

FIG. 34 (A)-(E) (FIG. 34) (A)-(E): In muscle, Lcn-2 treatment increased the expression (relative mRNA levels) of genes for UCP2 (mitochondrial uncoupling protein 2) as measured in four muscle: (A) Tibialis anterior; (B) Extensor digitorum longus; (C) soleus; (D) quadriceps; and (E) all four muscles combined measured at 50 ng/d/day, 150 ng/g/day, and 500 ng/g/day.

FIG. 35 (A)-(E) (FIG. 35) (A)-(E): In muscle, Lcn-2 treatment increased the expression (relative mRNA levels) of genes for myoG as measured in four muscle: (A) Tibialis anterior; (B) Extensor digitorum longus; (C) soleus; (D) quadriceps; and (E) all four muscles combined measured at 50 ng/d/day, 150 ng/g/day, and 500 ng/g/day.

FIG. 36 (A)-(E) (FIG. 36) (A)-(E): In muscle, Lcn-2 treatment increased the expression (relative mRNA levels) of genes for myoD as measured in four muscle: (A) Tibialis anterior; (B) Extensor digitorum longus; (C) soleus; (D) quadriceps; and (E) all four muscles combined measured at 50 ng/d/day, 150 ng/g/day, and 500 ng/g/day.

FIG. 37 (A)-(E) (FIG. 37) (A)-(E): In muscle, Lcn-2 treatment increased the expression (relative mRNA levels) of genes for myf-5 as measured in four muscle: (A) Tibialis anterior; (B) Extensor digitorum longus; (C) soleus; (D) quadriceps; and (E) all four muscles combined measured at 50 ng/d/day, 150 ng/g/day, and 500 ng/g/day.

FIG. 38 (A)-(E) (FIG. 38) (A)-(E): In muscle, Lcn-2 treatment increased the expression (relative mRNA levels) of genes for mrf-4 as measured in four muscle: (A) Tibialis anterior; (B) Extensor digitorum longus; (C) soleus; (D) quadriceps; and (E) all four muscles combined measured at 50 ng/d/day, 150 ng/g/day, and 500 ng/g/day.

FIG. 39 (FIG. 39): Lcn-2 treatment did not cause any inflammatory responses at concentrations 50 ng/g/day, 150 ng/g/day, and 500 ng/g/day as shown by the lack of any changes in relative mRNA expression levels of cytokines (A) TNFα; (B) IL-1α; (C) IL-1β; and (D) IL-6 in the liver.

FIG. 40 (FIG. 40): A decreased serum Lcn-2 level (serum BP2 ng/ml) was observed in mice with 50% osteoblast ablation.

FIG. 41 (FIG. 41): A decreased Lcn-2 expression on OCN+/CD146+ peripheral osteoblasts of type 2 diabetic patients was observed in comparison with healthy controls.

In the Summary of the Invention above, in the Detailed Description of the Invention, and the claims below, as well as the accompanying figures, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally. For the purposes of explanation, numerous specific details are set forth in order to provide a thorough understanding of the present invention. It will be apparent, however, to one skilled in the art that the present invention may be practiced without these specific details.

### DETAILED DESCRIPTION

It has now been discovered that Lcn-2 induces a dramatic increase in pancreatic cell proliferation and insulin production. Lcn-2 is also a potent regulator of myogenesis and muscle sensitivity. Administration of Lcn-2—both in vitro and in vivo—resulted in a dramatic increase in insulin 1 and insulin 2 production and secretion in Ins 1 pancreatic β cells and an increase in lean muscle mass, an increase in expression of genes promoting myogenesis, fatty acid oxidation and mitochondrial activity in muscle. These results have strong therapeutic implications for treating diseases or disorders associated with impaired glucose metabolism.

### DEFINITIONS

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lan, Antibodies: A Laboratory Manual, Cold Spring Harbor Laboratory Press, Cold Spring Harbor, N.Y. (1990); Principles of Neural Science, 4th ed., Eric R. Kandel, James H. Schwart, Thomas M. Jessell editors. McGraw-Hill/Appleton & Lange: New York, N.Y. (2000). Unless defined otherwise, all technical and scientific terms used herein have the same meaning as commonly understood by one of ordinary skill in the art.

The terms “individual” “subject” or “patient” are used interchangeably and means any mammalian subject for whom diagnosis, treatment, or therapy is desired, particularly humans. A “subject” as used herein generally refers to any living multicellular organism. Subjects include, but are not limited to animals (e.g., cows, pigs, horses, sheep, dogs, and cats) and plants, including hominoids (e.g., humans, chimpanzees, and monkeys). The term includes transgenic and cloned species. The term “patient” refers to both human and veterinary subjects.

The term “Administering” means “delivering in a manner which is affected or performed using any of the various methods and delivery systems known to those skilled in the art. Administering can be performed, for example, orally, or intravenously, via implant, transmucosally, transdermally, intradermally, intramuscularly, subcutaneously, or intraperitoneally. Administering can also be performed, for example, once, a plurality of times, and/or over one or more extended periods.

The terms “enumerated disease” or “enumerated disorders,” refer to type 1 or type 2 diabetes or glucose intolerance, obesity, obesity-related diseases, or metabolic syndrome and muscle diseases that are associated with reduced myogenesis or muscle mass, such as muscle atrophy, muscular dystrophy, polymyositis, myopathy, inflammatory muscle disease, MCAD and other fatty acid oxidation disorders. For example, carnitine/acylcarnitine translocase deficiency (CACT), carnitine palmitoyl transferase deficiency type II (CPT II), carnitine palmitoyl transferase deficiency type 1A (CPT1A), carnitine uptake defect (CUD), glutaric aciduria type II (GA II)/multiple acyl-CoA dehydrogenase deficiency (MADD), isobutyryl-CoA dehydrogenase deficiency (IBCD), medium chain acyl-CoA dehydrogenase deficiency (MCAD), long chain 3-hydroxyacyl-CoA dehydrogenase deficiency (LCHAD), short chain acyl-CoA dehydrogenase deficiency (SCAD), medium/short chain L-3-hydroxyacyl-CoA dehydrogenase deficiency (M/SCHAD), trifunctional protein deficiency (TFPD), and very long chain acyl-CoA dehydrogenase deficiency (VLCAD). “Enumerated disorders” further include conditions in which there is undesirably low insulin expression, insulin secretion, glucose metabolism, insulin sensitivity, Lcn-2 expression, and beta cell proliferation, such as prediabetic conditions.

The terms “therapeutically effective amount” or “an effective amount,” which are used interchangeably, mean an amount sufficient to mitigate, decrease or prevent the symptoms associated with the conditions disclosed herein, including diseases associated with diabetes, metabolic syndrome, obesity, and other related conditions contemplated for therapy with the compositions of the present invention. The phrases can mean an amount sufficient to produce a therapeutic result. Generally, the therapeutic result is an objective or subjective improvement of a disease or condition, achieved by inducing or enhancing a physiological process, blocking or inhibiting a physiological process, or in general terms performing a biological function that helps in or contributes to the elimination or abatement of the disease or condition. For example, eliminating or reducing or mitigating the severity of a disease or set of one or more symptoms. The full therapeutic effect does not necessarily occur by administration of one dose and may occur only after administration of a series of doses. Thus, a therapeutically effective amount further includes an amount effective to increase pancreatic β-cell proliferation, increase insulin expression, increase insulin sensitivity, increase glucose tolerance, decrease weight gain, decrease fat mass, increase weight loss, increase bone mass, increase adiponectin serum levels, and decrease serum resistin levels.

“Treating” a disease means taking steps to obtain beneficial or desired results, including clinical results, such as mitigating, alleviating or ameliorating one or more symptoms of a disease; diminishing the extent of disease; delaying or slowing disease progression; ameliorating and palliating or stabilizing a metric (statistic) of disease; causing the subject to experience a reduction, delayed progression, regression or remission of the disorder and/or its symptoms. “Treatment” refers to the steps taken. In one embodiment, recurrence of the disorder and/or its symptoms is prevented. In the preferred embodiment, the subject is cured of the disorder and/or its symptoms. “Treatment” or “treating” can also refer to therapy, prevention and prophylaxis and particularly refers to the administration of medicine or the performance of medical procedures with respect to a patient, for either prophylaxis (prevention) or to cure (if possible) or reduce the extent of or likelihood of occurrence of the infirmity or malady or condition or event in the instance where the patient is afflicted. More particularly, as related to the present invention, “treatment” or “treating” is defined as the application or administration of a therapeutic agent to a patient, or application or administration of a therapeutic agent to an isolated tissue or cell line from a patient, who has a disease, a symptom of disease or a predisposition toward development of a disease. Treatment can slow, cure, heal, alleviate, relieve, alter, mitigate, remedy, ameliorate, improve or affect the disease, a symptom of the disease or the predisposition toward disease. In the present invention, the treatments using the agents described may be provided to prevent diabetes, metabolic syndrome, and obesity or obesity-related diseases. More preferably, the goal is the treatment of type 1 or type 2 diabetes including disorders or complications associated with them.

The term “mitigating” means reducing or ameliorating a disease or symptom of a disease. For example, mitigation can be achieved by administering a therapeutic agent before the phenotypic expression of the disease (i.e., prior to the appearance of symptoms of the disease). Mitigation includes making the effects of diseases less severe by avoiding, containing, reducing or removing it or a symptom of it. Mitigating an enumerated disease as described herein comes within the definition of “treating” an enumerated disease before symptoms occur. Amounts of therapeutic agents that mitigate a disease are herein referred to as “therapeutically effective amounts.”

“cDNA” or “complementary DNA” is DNA synthesized from a messenger RNA (mRNA) template in a reaction catalyzed by the enzyme reverse transcriptase and the enzyme DNA polymerase. In this application cDNA can be used to generate antisense nucleic acids for use in reducing expression of FoxO1.

“Antisense oligonucleotides” are single strands of DNA or RNA that are complementary to a chosen sequence. In the case of antisense RNA they prevent protein translation of certain messenger RNA strands by binding to them. Antisense DNA can be used to target a specific, complementary (coding or non-coding) RNA. If binding takes places this DNA/RNA hybrid can be degraded by the enzyme RNase H.

“Small interfering RNA” or “siRNA”—sometimes known as short interfering RNA or silencing RNA—is a class of double-stranded RNA molecules, 20-25 nucleotides in length, that play a variety of roles in biology. The most notable role of siRNA is its involvement in the RNA interference (RNAi) pathway, where it interferes with the expression of a specific gene.

“MiR” also “micro RNA” means a newly discovered class of small non-coding RNAs that are key negative regulators of gene expression. Like conventional protein-encoding RNA, miRs are transcribed by RNA polymerase II and their expression is controlled by transcriptional factors. The mature miRs inhibit target mRNA translation or promote their degradation by directly binding to specific miR binding sites in the 3′-untranslated region (3′-UTR) of target genes.

The term “Lipocalin-2” (Lcn-2) also known as oncogene 24p3 or neutrophil gelatinase-associated lipocalin (NGAL), is a protein that in humans is encoded by the Lcn-2 gene. The Lipocalin family comprises a diverse group of mostly secreted soluble proteins that bind hydrophobic ligands and act as transporters, carrying small molecules to specific cells. Lipocalins are related by possessing an 8-stranded β-barrel structure. Lcn-2 is a component of granules in neutrophils from tissues that are normally exposed to microorganisms and is upregulated during inflammation. Lcn-2 can form homodimers and can heterodimerize with the neutrophil gelatinase MMP-9.

As used herein, a “bone loss disorder” refers to a loss of bone density, either localized or non-specific. “Osteopenia” in the context of this invention refers to general loss of bone density below normal, where the bone loss is not site-specific. “Osteoporosis” is a type of osteopenia where bone loss is more advanced and is diagnosed based on common clinical standards. “Diabetes” refers to high blood sugar or ketoacidosis, as well as chronic, general metabolic abnormalities arising from a prolonged high blood sugar status or a decrease in glucose tolerance. “Diabetes” encompasses both type 1 and type 2 (Non Insulin Dependent Diabetes Mellitus or NIDDM) forms of the disease. The risk factors for diabetes include the following factors: waistline of more than 40 inches for men or 35 inches for women, blood pressure of 130/85 mmHg or higher, triglycerides above 150 mg/dl, fasting blood glucose greater than 100 mg/dl or high-density lipoprotein of less than 40 mg/dl in men or 50 mg/dl in women.

The term “insulin resistance” (IR) means a state in which a normal amount of insulin produces a less than normal biological response relative to the biological response in a subject that does not have insulin resistance. It is a physiological condition in which the natural hormone insulin becomes less effective at lowering blood sugar. The resulting increase in blood glucose may raise blood glucose levels outside of their normal range and cause adverse health effects, depending on dietary conditions. Insulin resistance normally refers to reduced glucose-lowering effects of insulin. However, other functions of insulin can also be affected. For example, insulin resistance in fat cells reduces the normal effects of insulin on lipids and results in reduced uptake of circulating lipids and increased hydrolysis of stored triglycerides. Increased mobilization of stored lipids in these cells elevates free fatty acids in the blood plasma. Elevated blood fatty-acid concentrations (associated with insulin resistance and diabetes mellitus type 2), reduced muscle glucose uptake, and increased liver glucose production all contribute to elevated blood glucose levels. High plasma levels of insulin and glucose due to insulin resistance are a major component of the metabolic syndrome. If insulin resistance exists, more insulin needs to be secreted by the pancreas. If this compensatory increase does not occur, blood glucose concentrations increase and type 2 diabetes occurs.

The term “insulin sensitivity” refers to tissue responsiveness to insulin, meaning how successfully the receptor operates to permit glucose clearance. An “insulin resistance disorder” as discussed herein, refers to any disease or condition that is caused by or contributed to by insulin resistance. Examples include: diabetes, obesity, metabolic syndrome, insulin-resistance syndromes, syndrome X, insulin resistance, high blood pressure, hypertension, high blood cholesterol, dyslipidemia, hyperlipidemia, dyslipidemia, atherosclerotic disease including stroke, coronary artery disease or myocardial infarction, hyperglycemia, hyperinsulinemia and/or hyperproinsulinemia, impaired glucose tolerance, delayed insulin release, diabetic complications, including coronary heart disease, angina pectoris, congestive heart failure, stroke, cognitive functions in dementia, retinopathy, peripheral neuropathy, nephropathy, glomerulonephritis, glomerulosclerosis, nephrotic syndrome, hypertensive nephrosclerosis some types of cancer (such as endometrial, breast, prostate, and colon), complications of pregnancy, poor female reproductive health (such as menstrual irregularities, infertility, irregular ovulation, polycystic ovarian syndrome (PCOS)), lipodystrophy, cholesterol related disorders, such as gallstones, cholecystitis and cholelithiasis, gout, obstructive sleep apnea and respiratory problems, osteoarthritis, and prevention and treatment of bone loss, e.g., osteoporosis.

“Glucose Intolerance” is a pre-diabetic state in which the blood glucose is higher than normal but not high enough to warrant the diagnosis of diabetes.

“Metabolic Syndrome” means a disease characterized by spontaneous hypertension, dyslipidemia, insulin resistance, hyperinsulinemia, increased abdominal fat and increased risk of coronary heart disease. As used herein, “metabolic syndrome” shall mean a disorder that presents risk factors for the development of type 2 diabetes mellitus and cardiovascular disease and is characterized by insulin resistance and hyperinsulinemia and may be accompanied by one or more of the following: (a) glucose intolerance, (b) type 2 diabetes, (c) dyslipidemia, (d) hypertension and (e) obesity.

“Obesity” means a condition in which the body weight of a mammal exceeds medically recommended limits by at least about 20%, based upon age and skeletal size. “Obesity” is characterized by fat cell hypertrophy and hyperplasia. “Obesity” may be characterized by the presence of one or more obesity-related phenotypes, including, for example, increased body mass (as measured, for example, by body mass index, or “BMI”), altered anthropometry, basal metabolic rates, or total energy expenditure, chronic disruption of the energy balance, increased fat mass as determined, for example, by DEXA (Dexa Fat Mass percent), altered maximum oxygen use (VO2), high fat oxidation, high relative resting rate, glucose resistance, hyperlipidemia, insulin resistance, and hyperglycemia. See also, for example, Hopkinson et al. (1997) Am J Clin Nutr 65(2): 432-8 and Butte et al. (1999) Am J Clin Nutr 69(2): 299-307. “Overweight” individuals are generally having a body mass index (BMI) between 25 and 30. “Obese” individuals or individuals suffering from “obesity” are generally individuals having a BMI of 30 or greater. Obesity may or may not be associated with insulin resistance.

An “obesity-related disease” or “obesity related disorder” or “obesity related condition,” which are all used interchangeably, refers to a disease, disorder, or condition, which is associated with, related to, and/or directly or indirectly caused by obesity, including coronary artery disease/cardiovascular disease, hypertension, cerebrovascular disease, stroke, peripheral vascular disease, insulin resistance, glucose intolerance, diabetes mellitus, hyperglycemia, hyperlipidemia, dyslipidemia, hypercholesteremia, hypertriglyceridemia, hyperinsulinemia, atherosclerosis, cellular proliferation and endothelial dysfunction, diabetic dyslipidemia, HIV-related lipodystrophy, peripheral vessel disease, cholesterol gallstones, cancer, menstrual abnormalities, infertility, polycystic ovaries, osteoarthritis, sleep apnea, metabolic syndrome (Syndrome X), type H diabetes, diabetic complications including diabetic neuropathy, nephropathy, retinopathy, cataracts, heart failure, inflammation, thrombosis, congestive heart failure, and any other cardiovascular disease related to obesity or an overweight condition and/or obesity related asthma, airway and pulmonary disorders.

“Osteocalcin,” also known as bone gamma-carboxyglutamic acid-containing protein (BGLAP), is a noncollagenous protein found in bone and dentin. In humans, the osteocalcin is encoded by the BGLAP gene. Osteocalcin is secreted solely by osteoblasts and thought to play a role in the body's metabolic regulation and is pro-osteoblastic, or bone-building, by nature. It is also implicated in bone mineralization and calcium ion homeostasis. Osteocalcin acts as a hormone in the body, causing β cells in the pancreas to release more insulin, and at the same time directing fat cells to release the hormone adiponectin, which increases sensitivity to insulin.

An individual “at risk” may or may not have detectable disease, and may or may not have displayed detectable disease prior to the treatment methods described herein. “At risk” denotes that an individual who is determined to be more likely to develop a symptom based on conventional risk assessment methods or has one or more risk factors that correlate with development of diabetes, metabolic syndrome, or obesity or an obesity-related disease, or a disease for which Lcn-2 administration provides a therapeutic benefit. An individual having one or more of these risk factors has a higher probability of developing diabetes, metabolic syndrome, obesity, or an obesity-related disease, than an individual without these risk factors. Examples (i.e., categories) of risk groups are well known in the art and discussed herein.

“FoxO1” or Forkhead box protein O1, also known as forkhead in rhabdomyosarcoma, is a protein that in humans is encoded by the FoxO1 gene. is a transcription factor that plays important roles in regulation of gluconeogenesis and glycogenolysis by insulin signaling, and is also central to the decision for a preadipocyte to commit to adipogenesis. It is primarily regulated through phosphorylation on multiple residues; its transcriptional activity is dependent on its phosphorylation state.

A “peroxisome proliferator activated receptor” or “PPAR” is a member of a family of nuclear receptors, distinguished in alpha (α), delta (δ), and gamma (γ) subtypes as described herein. As used herein the term “PPAR” refers to a peroxisome proliferator-activated receptor as recognized in the art. As indicated above, the PPAR family includes PPARα (also referred to as PPARα or PPARalpha), PPARδ (also referred to as PPARd or PPARdelta), and PPARγ (also referred to as PPARg or PPARgamma). PPARs are members of the nuclear receptor superfamily, that function as ligand-regulated nuclear transcription factors that control the expression of target encodingenzymes involved in lipid metabolism and differentiation of adipocytes.

The concept of “combination therapy” means administering two or more agents that target the same pathogen or biochemical pathway sometimes results in greater efficacy and diminished side effects relative to the use of the therapeutically relevant dose of each agent alone. Additive or synergistic effects can be achieved. The two compounds can be delivered simultaneously, e.g. concurrently, or sequentially.

Resistin also known as adipose tissue-specific secretory factor (ADSF) or C/EBP-epsilon-regulated myeloid-specific secreted cysteine-rich protein (XCP1) is a cytokine found to be produced and released from adipose tissue and serves endocrine functions likely involved in insulin resistance.

A “kit” is any manufacture (e.g., a package or container) comprising at least one reagent, e.g., a medicament for treatment of a disease, or a probe for specifically detecting a biomarker gene or protein of the invention. In certain embodiments, the manufacture is promoted, distributed, or sold as a unit for performing the methods of the present invention.

“Therapeutic agents” and “Active agents” are used interchangeably and include Lcn-2, insulin (preferably recombinant human insulin), incretins, sulfonylureas, meglitinides, D-Phenylalanine Derivatives (nateglinides), biguanides, thiazolidinediones, alpha-glucose inhibitors, GLP-1, GLP-1 analogues such as liraglutide, exendin-4 LY5448806 and CJC-1131, as well as dipeptidyl peptidase N inhibitors. Sulfonylureas are exemplified by glimepiride, glyburide, chlorpropamide, acetohexamide, glipizide, tolbutamide, and tolazamide. Meglitinides are exemplified by Repaglinide. Biguanides are exemplified by metformin and metformin hydrochloride. Thiazolidinediones are exemplified by pioglitazone and rosiglitazone. Other agents include anti-coagulants, vasodilators, drugs used to treat atherosclerosis, vitamin K inhibitors, statins, and beta blockers.

In the following description, for the purposes of explanation, numerous specific details are set forth in order to provide a thorough understanding of the present invention.

It will be apparent, however, to one skilled in the art that the present invention may be practiced without these specific details. In other instances, well-known structures and devices are shown in block diagram form in order to avoid unnecessarily obscuring the present invention.

### BACKGROUND

Skeletal functions have recently been shown to expand beyond regulation of bone mass homeostasis to affect whole body physiology. In particular the skeleton has been shown to regulate energy metabolism³⁻⁵ and hematopoiesis. In its metabolic functions the skeleton, through the osteoblast-secreted hormone osteocalcin, favors n-cell proliferation, insulin secretion and sensitivity and energy expenditure. In the hematopoietic system, osteoblasts maintain the hematopoietic stem cell pool and their dysfunction can induce neoplastic changes in heterotypic cells, supporting a concept of niche-driven oncogenesis. It has now been discovered that the osteoblast-expressed protein, Lcn-2, induced a dramatic increase in pancreatic cell proliferation and insulin production. Lcn-2 was also discovered to be a potent regulator of myogenesis and muscle sensitivity. Administration of Lcn-2-both in vitro and in vivo—resulted in a dramatic increase in insulin 1 and insulin 2 production and secretion in Ins 1 pancreatic β cells and an increase in lean muscle mass, an increase in expression of genes promoting myogenesis, fatty acid oxidation and mitochondrial activity. Lcn-2 is preferentially expressed (i) in bone as compared to white adipose tissue; (ii) in osteoblasts as compared to adipocytes; and (iii) in bone marrow derived stromal cells as compared to adipocytes. Diseases associated with impaired glucose metabolism, such as diabetes, metabolic syndrome, and obesity can be both treated (mitigated and prevented) by administering Lcn-2.

As will be described in detail, ablation of osteoblasts in adult mice profoundly affected glucose metabolism. A partial ablation of this cell population resulted in hypoinsulinemia, hyperglycemia, glucose intolerance, and decreased insulin sensitivity, as is the case in osteocalcin deficiency. However, and unlike what is seen in osteocalcin-deficient mice, osteoblast ablation also decreased gonadal fat, increased energy expenditure and food intake, and increased the expression of resistin, an adipokine proposed to mediate insulin resistance. While, administration of osteocalcin reversed, fully, the glucose intolerance and reinstated normal blood glucose and insulin levels, it only partially restored insulin sensitivity and did not affect the improved gonadal fat weight and energy expenditure in osteoblast-depleted mice.

Without being bound by theory, these observations not only strengthen the notion that osteoblasts are necessary for glucose homeostasis and energy expenditure, but they also suggest that in addition to osteocalcin, other osteoblast-derived hormones such as Lcn-2, contribute to the function of the skeleton as a regulator of energy metabolism. Indeed osteoblasts may affect insulin signaling and glucose-regulating functions of pancreas, liver, white adipose tissue, and muscle by a multifactorial process mediated by the actions of more than one osteoblast-derived hormone that could act either in synergy with osteocalcin, or by counteracting osteocalcin in some of its metabolic functions.

The results of experiments described below show that the osteoblast-derived hormone Lcn-2 performs a wide-range of actions including an increase and/or decrease in expression of genes and proteins in myocytes, INS-1 cells, adipocytes, adipose tissue, muscle, osteoblasts and liver. These actions ultimately improve glucose tolerance, increase insulin, increase insulin sensitivity, decrease fat mass, increase lean mass, increase energy expenditure and activity, and decrease food intake. The overall outcome and the purpose of these interactions is glucose homeostasis, which in turn also increased myogenesis. Because administration of Lcn-2 showed a dramatic improvement in glucose tolerance and improved insulin sensitivity, certain embodiments of the invention are directed to methods for treating and preventing diseases associated with impaired glucose metabolism such as diabetes, metabolic syndrome, and obesity, or obesity-related diseases.

### Embodiments of the Invention

Diseases such as diabetes, metabolic syndrome, and obesity, or obesity-related diseases are a definitive consequence of impaired glucose metabolism. In an effort to investigate regulation of glucose metabolism, glucose sensitivity, and glucose tolerance, these conditions have been reproduced in both in vitro and in vivo models. With the use of these models, it has now been discovered that the enumerated diseases affecting glucose metabolism can be both treated and prevented by administration of the osteoblast-secreted hormone Lcn-2.

The animal studies described herein, involved Lcn-2 administered intraperitoneally to mice at 50, 150 and 500 micrograms/kg (equivalent to ng) body weight. The most effective amount was 150 micrograms/kg in mice. The therapeutically effective amount in humans is not necessarily the same as in mice, however, extrapolating the animal dose to a human dose would generate a range of from about 4 to about 60 micrograms/kg (about 4 to about 40 micrograms/kg for adults and 6 to 60 micrograms/kg for children who have a higher metabolic rate and therefore typically receive correspondingly higher amounts). The range of effective doses in humans can be still broader than this depending on factors such as the type of disease, the severity of the disease, the route of administration and the formulation, as is discussed below. The therapeutically effective amount is an amount that significantly increases serum Lcn-2 levels. “Significantly increases” or “significantly higher” is at least about a 15% increase over control levels or pretreatment levels. Similarly, “significantly decreases” or “significantly lower” is at least about a 15% decrease over control levels or pretreatment levels.

As is described in the Summary of the Invention, based on the ability of Lcn-2 to induce a dramatic increase in insulin production by pancreatic cells, some embodiments are directed to methods for identifying a subject having or at risk of developing a disorder selected from the group consisting of type 1 or type 2 diabetes, metabolic syndrome, obesity or obesity-related disease, and administering a therapeutically effective amount of Lcn-2 or a biologically active fragment or variant thereof (hereafter collectively “Lcn-2”), either alone or in combination with other active agents known to treat these conditions to treat or prevent the disorder. Lcn-2 is administered in an amount effective to produce an effect selected from the group consisting of an increase in pancreatic β-cell proliferation, an increase in insulin expression, an increase in insulin secretion, an increase in insulin sensitivity, an increase in glucose tolerance, a decrease in weight gain, a decrease in fat mass, an increase in weight loss and an increase in serum adiponectin levels, or a decrease in serum resistin levels as compared to pretreatment levels. Preferably, the subject in all methods described herein is a human. In certain embodiments, dose ranges of Lcn-2 are for use in humans is from about 4 to 60 micrograms/kg (from about 4 to 40 micrograms/kg for adults and 6 to 60 micrograms/kg for children). The dosage range is higher because children have higher metabolic rates. The formula used to calculate conversions between species includes a variant for metabolic rates.

In other embodiments of the invention, methods are directed to administering Lcn-2 or a biologically active fragment or variant to a subject in an amount that causes an effect selected from the group consisting of a significant increase in pancreatic β-cell proliferation, insulin expression, insulin sensitivity, glucose tolerance, a decrease weight gain, a decrease in fat mass, an increase weight loss, an increase in bone mass, an increase in adiponectin serum levels, and a decrease in serum resistin levels. The subject can be a human.

Formulations of Lcn-2 alone or with other active agents are described below in detail. Where disorders of glucose metabolism such as diabetes, metabolic syndrome, and obesity, or obesity-related diseases are being treated with a therapeutically effective amount of Lcn-2 or a biologically active fragment or variant in a subject, an embodiment of the method further comprises co-administration of one or more agents (e.g., anti-diabetic drugs) or drugs to treat complications or conditions associated with diabetes either in the same or in separate pharmaceutical compositions and kits, either on the same day or on different days. Certain embodiments are directed to formulations of Lcn-2 alone or with other active agents (such as anti-diabetic drugs) that have a therapeutic use to treat or prevent one or more of the disorders described. Such agents include anti-coagulants, vasodilators, drugs used to treat atherosclerosis, vitamin K inhibitors, statins, beta blockers, in amounts effective to provide therapeutic benefit of the drug in the combination therapy. Therapeutically effective amounts of Lcn-2 or a biologically active fragments or variants may be administered with resistin, adiponectin, and therapeutic oligonucleotides that reduce the expression or biological activity of FoxO1.

In certain embodiments of the invention, the therapeutic amount produces an effect selected from the group consisting of a significant increase in pancreatic β-cell proliferation, insulin expression, insulin sensitivity, glucose tolerance, a decrease weight gain, a decrease in fat mass, an increase weight loss, an increase in bone mass, an increase in adiponectin serum levels, and a decrease in serum resistin levels. The subject can be a human.

Anti-diabetic agents that can be formulated or administered in combination with Lcn-2 include for example, insulin (preferably recombinant human insulin), incretins, sulfonylureas, meglitinides, D-Phenylalanine Derivatives (nateglinides), biguanides, thiazolidinediones, alpha-glucose inhibitors, GLP-1, GLP-1 analogues such as liraglutide, exendin-4 LY5448806 and CJC-1131, as well as dipeptidyl peptidase IV inhibitors. Sulfonylureas are exemplified by glimepiride, glyburide, chlorpropamide, acetohexamide, glipizide, tolbutamide, and tolazamide. Meglitinides are exemplified by Repaglinide. Biguanides are exemplified by metformin and metformin hydrochloride. Thiazolidinediones are exemplified by pioglitazone and rosiglitazone. Other agents that treat diabetes include (nateglinides), biguanides, thiazolidinediones, alpha-glucose inhibitors, GLP-1, GLP-1 analogues such as liraglutide, exendin-4 LY5448806 and CJC-1131, as well as dipeptidyl peptidase IV inhibitors.

Another embodiment is directed to a method comprising identifying a subject having or at risk of developing type 1 or type 2 diabetes, metabolic syndrome, obesity, or obesity-related diseases and administering to the subject a therapeutically effective amount of an agent that reduces Forkhead Box Protein O1 (FoxO1) expression or FoxO1 activity (either alone or together with Lcn-2), preferably in osteoblasts, in an amount that significantly increases serum Lcn-2 levels. We have previously reported that the transcription factor, FoxO1, acts on osteoblasts to regulate whole body glucose metabolism (Rached et al. J. Clinical Investigation, 2010). In certain embodiments the active agent for reducing FoxO1 expression or activity in this method is an isolated nucleic acid selected from the group comprising antisense DNAs, antisense RNAs, micro RNAs (miRNAs), ribozymes, and small interfering RNAs (siRNAs). For example, the miRNA may be miR-182 that targets Fox01 specifically in osteoblasts (Kim K. M. et al. J Bone Mineral Research 2012) and T-cells, et al. Nature Immunology 2010). Other non-osteoblast-specific microRNAs for use in the present methods include miR-96 and miR-183 (Xie L et al Blood 2012), miR-135b (Matsuyama H. Blood 2011). Expression of miR-9, miR-27, miR-96, miR-153, miR-182, miR-183, or miR-186, but not miR-29a, miR-128, miR-152, or miR-486 mimetics in HEC-1B cells were sufficient to significantly reduce the abundance of FoxO1. The miRNA can be selected from the group consisting of miR-182, miR-96, miR-183, and miR-135b.

Other embodiments are directed to methods comprising identifying a subject having or at risk of developing a disorder of the bone in a subject by administering therapeutically effective amounts of Lcn-2 or biologically active fragments or variants, or an inhibitory oligonucleotide that reduces Fox01 expression or FoxO1 activity. Such methods are effective in treating or preventing the bone disorder. The bone disorder includes osteoporosis, osteopenia, osteomyelitis, and osteoarthritis. Bone disorders may be the result of bone loss due to any disease or treatment for disease causing bone loss, including, but not limited to treatment for cancer.

In certain other embodiments, methods are directed to identifying a subject having or at risk of developing a muscle disorder and administering a therapeutically effective amount of Lcn-2 or a biologically active fragment or variant and/or or an inhibitory oligonucleotide that reduces FoxO1 expression or activity to the subject in an amount that increases or maintain myogenesis and thereby treats or prevents the muscle disorder. The muscle disorder is selected from the group consisting of muscle atrophy, muscular dystrophy, fibromyalgia, myositis, polymyositis, myopathy, rhabdomyolysis, inflammatory muscle disease, MCAD, and other fatty acid oxidation disorders and caritine/acylcarnitine translocase deficiency or CACT.

Muscle tissue may be compromised by diseases and disorders such as but not limited to muscular dystrophy and muscular atrophy, for example due to excessive irradiation in cancer treatment, polymyositis, myopathy, rhabdomyolysis, inflammatory muscle disease, MCAD and other fatty acid oxidation disorders. For example, carnitine/acylcarnitine translocase deficiency (CACT), carnitine palmitoyl transferase deficiency type II (CPT II), carnitine palmitoyl transferase deficiency type 1A (CPT1A), carnitine uptake defect (CUD), glutaric aciduria type II (GA II)/multiple acyl-CoA dehydrogenase deficiency (MADD), isobutyryl-CoA dehydrogenase deficiency (IBCD), medium chain acyl-CoA dehydrogenase deficiency (MCAD), long chain 3-hydroxyacyl-CoA dehydrogenase deficiency (LCHAD), short chain acyl-CoA dehydrogenase deficiency (SCAD), medium/short chain L-3-hydroxyacyl-CoA dehydrogenase deficiency (M/SCHAD), trifunctional protein deficiency (TFPD), and very long chain acyl-CoA dehydrogenase deficiency (VLCAD).

Other embodiments of the invention include methods for increasing beta-cell area or beta-cell numbers in pancreatic cells in a subject in need, by contacting pancreatic beta cells in vivo or in vitro with a therapeutically effective amount of an agent. Preferably, the agent is Lcn-2 or a biologically active fragment or variant. The beta cells are human cells.

### Overview

As it stands, current treatments for diabetes do not represent a cure. A dominant feature of diabetes is impaired β-cell function. One abnormality that occurs early in the disease progression in both type 1 diabetes and 2 diabetes, is the loss of eating-induced rapid insulin response. This loss causes the liver to continue to produce glucose, in addition to the glucose that is ingested and absorbed from the basic components of a meal. Type 2 diabetics typically exhibit a delayed response to increases in blood glucose levels. Normal individuals usually begin to release insulin within 2-3 minutes following the consumption of food. But type 2 diabetics may not secrete endogenous insulin until blood glucose begins to rise. As a result, endogenous glucose production is not shut off and continues after consumption. The patient then experiences hyperglycemia (elevated blood glucose levels). Another characteristic of type 2 diabetes is impaired insulin action, termed insulin resistance. To handle a given glucose load, more insulin is required and that increased insulin concentration must be maintained for a longer period of time. Consequently, the diabetic patient is also exposed to elevated glucose concentrations for prolonged periods of time, which further aggravates insulin resistance. Ultimately, prolonged elevated blood glucose levels are themselves toxic to β cells. See Richardson, et al., U.S. Pat. No. 8,119,593. Genetic defects can also play a role in β-cell deterioration (Clee, S. M., et al. Nature Genetics 38:688-693, 2006). Eventually, the pancreas becomes overwhelmed, and eventually individuals progress to develop insulin deficiency similar to people with type 1 diabetes. See Richardson et al., U.S. Pat. No. 8,119,593.

Insulin therapy is the standard protocol for treatment of type 1 diabetes. While type2 diabetes can be treated with diet and exercise, most early stage type 2 diabetics are currently treated with oral antidiabetic agents. This treatment is met with limited success. Patients generally transition to insulin therapy as the disease progresses. In a typical progression, the first oral antidiabetic agent used is metformin. Metformin is a suppressor of hepatic glucose output. The use of metformin is not associated with weight gain or hypoglycemia. If metformin treatment is insufficient to control hyperglycemia, an insulin secretagogue, usually a sulfonylurea, can be added to the treatment regimen. Secretagogues raise the basal level of insulin in order to lower average blood glucose levels. The use of sulphonylureas is associated with weight gain and can lead to hypoglycemia; however, severe hypoglycemia is unusual. If this combination of two oral antidiabetic agents is inadequate to control hyperglycemia either a third oral agent, such as a glitazone, or a long-acting, basal insulin can be added to the regimen. As the disease progresses, insulin therapy can be intensified by the addition of intermediate and short (rapid) acting insulin preparations administered in association with at least some of the day's meals. See Richardson, et al., U.S. Pat. No. 8,119,593.

Metabolic syndrome consists of a collection of health risks that increase the chance of an individual developing heart disease, stroke and diabetes. Metabolic syndrome is not a disease in and of itself. Instead, the name is given to a cluster of metabolic disorders including high blood pressure, high insulin levels, excess body weight, and abnormal cholesterol levels. Each or these conditions is considered to be a risk factor for certain other diseases, however, combined together, these conditions indicate a significantly higher likelihood of developing a life threatening disease. According to some surveys, more than one in five Americans has metabolic syndrome with a greater preponderance of the syndrome present in people of higher age. See Dobak, John D. U.S. Pat. No. 8,145,299.

The indicators of metabolic syndrome include obesity. Specifically, obesity is located around the waist. A waistline of 40 inches or more for men and 35 inches or more for women would qualify. Another indicator is high blood pressure such as a blood pressure of 130/85 mmHg or greater. Yet another factor is one or more abnormal cholesterol levels including a high density lipoprotein level (HDL) less than 40 mg/dl for men and under 50 mg/dl for women. A triglyceride level above 150 mg/dl may also be an indicator. Finally, a resistance to insulin is an indicator of metabolic syndrome which may be indicated by a fasting blood glucose level greater than 100 mg/dl. See Dobak, John D., U.S. Pat. No. 8,145,299; see also, “Assessing Your Weight and Health Risk” (website published by the National Institutes of Health) and “What is Metabolic Syndrome?” (2007 publication by the American Heart Association)

According to the American Heart Association, three groups of people are often afflicted with metabolic syndrome. The first group includes people with diabetes who cannot maintain a proper glucose level. The second group includes people without diabetes who have high blood pressure and also secrete large amounts of insulin to maintain glucose levels (hyperinsulinemia). Finally, a third group includes people who have survived a heart attack and have hyperinsulinemia without glucose intolerance. See Dobak, John D., U.S. Pat. No. 8,145,299; see also, “What is Metabolic Syndrome?” (2007 Publication by the American Heart Association).

The fundamental cause of metabolic syndrome is thought to be insulin resistance. Insulin loses its ability to make one's body cells absorb glucose from the blood. When this happens, glucose levels remain high after eating. As a result, the pancreas begins to excrete insulin in response to high glucose levels. The body then reacts to this condition by stimulating the pancreas to generate more and more insulin. This generation of more insulin is in an effort to achieve a normal level of glucose absorption. Ultimately, the pancreas cannot keep up the levels of insulin necessary to maintain proper glucose absorption. Glucose accumulates in the blood leading to type 2 diabetes.

High levels of insulin and glucose may cause a variety of negative effects such as damage to the lining of arteries which can lead to heart attack or stroke. These abnormal levels can also cause changes in the ability of the kidneys to remove salt, leading to high blood pressure, heart disease and stroke. Other consequences include an increase in triglyceride levels. Elevated triglyceride levels can lead to an increased risk of developing cardiovascular disease as well as a slowing of insulin production, which can indicate the onset of type 2 diabetes, which in turn can cause heart attack, stroke, as well as damage to the eyes, nerves or kidneys. See Dobak, John D., U.S. Pat. No. 8,145,299.

Obesity, which is defined in general terms as an excess of body fat relative to lean body mass, is now a world-wide epidemic, and is one of the most serious contributors to increased morbidity and mortality. Obesity causes metabolic abnormalities such as insulin resistance and Type 2 diabetes (non-insulin-dependent diabetes mellitus (NIDDM)), hyperlipidemia, and endothelial dysfunction. These abnormalities predispose the vasculature to injury, cellular proliferation and lipid oxidation, with resulting atherosclerosis leading to heart attack, stroke, and peripheral vascular diseases.

As described above, the prevalence and seriousness of diabetes, metabolic syndrome, and obesity or obesity-related diseases are cause for concern. A need exists for a better understanding of how the body regulates glucose homeostasis so that new therapies and therapeutic agents to treat and prevent these diseases associated with impaired glucose metabolism can be developed.

### SUMMARY OF RESULTS

The results herein show that administration of therapeutically effective amounts of Lcn-2 can treat and prevent diseases in a mammal that are related to impaired glucose metabolism, such as diabetes, metabolic syndrome, obesity, and obesity-related disorders, and Lcnn-2 can increase or maintain myogenesis. The following is a summary of results of experiments described in the Examples:

Lcn-2 Exhibits Preferential Expression in Bone.

1. Lcn-2 expression is upregulated in the femur of 1-month old female FoxO1_{osb}−/− mice;
2. Lcn-2 serum levels increase in FoxO1_{osb}−/− mice of different genetic backgrounds;
3. Lcn-2 is preferentially expressed in the bone as compared to white adipose tissue;
4. Lcn-2 is preferentially expressed in osteoblasts as compared to adipocytes;
5. Lcn-2 is preferentially expressed in bone marrow derived stromal cells as compared to adipocytes; and
6. Lcn-2 is an osteoblast-specific secreted molecule; these results support methods that target inhibitory FoxO1 oligonucleotides to osteoblasts as a way of increasing endogenous Lcn-2 expression in a subject having an enumerated disease.

Lcn-2 Exhibits a Wide-Range of Action on Expression of Genes and Proteins in Certain Cells, Tissues, and Organs.

7. Recombinant Lcn-2 increases Ins1 and Ins2 expression in INS-1 cells;
8. Recombinant Lcn-2 increases expression of cyclin D2 and cdk-4 in INS-1 cells;
9. Recombinant Lcn-2 increases expression of PPARα in C2C12 myocytes;
10. Recombinant Lcn-2 increases expression of PGC-1a in C2C12 myocytes;
11. Recombinant Lcn-2 increases expression of Nrf-1 in C2C12 monocytes;
12. Recombinant Lcn-2 increases expression of Mcad in C2C12 myocytes;
13. Recombinant Lcn-2 increases expression of Mrf-4 in C2C12 myocytes;
14. Recombinant Lcn-2 increases expression of Adiponectin in 3T3-L1 adipocytes;
15. Recombinant Lcn-2 decreases the expression of Resistin in 3T3-L1 adipocytes;
16. Lcn-2 improves glucose tolerance, increases insulin, increases insulin sensitivity, decreases fat mass, increases lean mass, increases energy expenditure and activity, and decreases food intake, which supports those methods that treat an enumerated disease by administering Lcn-2, or an agent that reduces expression of FoxO1 preferably in osteoblasts;
17. Lcn-2 suppresses the expression of Tgl, Perillipin, Lpl, C/EBPα, and PPARγ genes in white adipose tissue;
18. Lcn-2 treated mice demonstrated a decrease in expression of RBP-4;
19. Lcn-2 increases expression of acylcoA, the first and rate-limiting enzyme in the peroxisomal fatty acid β-oxidation pathway;
20. Lcn-2 increases expression of Nrf-1 and Mcad in muscle;
21. Lcn-2 increases expression of UCP2;
22. Lcn-2 treatment upregulates all key transcription factors such as myoG, myoD, myf-5, and mrf-4 in muscle. This result plus the evidence that Lcn-2 increases insulin sensitivity support methods for treating muscle diseases associated with reduced muscle mass or myogenesis by administering Lcn-2;
23. Lcn-2 treatment failed to cause any inflammatory responses shown by a lack of change in expression of cytokines TNF α, IL-1α, IL-1β, and IL-6 in liver. This result supports the therapeutic use of Lcn-2 as it does not cause an adverse inflammatory side effect;
24. Serum levels of Lcn-2 decreased 50% in mice lacking 50% of osteoblasts; and
25. Lcn-2 expression was significantly decreased in osteoblasts obtained from diabetic patients.

Details of these results are set forth below and are also described in the Examples.

### Therapeutic Oligonucleotides

Certain embodiments of the present invention encompass the use of inhibitory oligonucleotides such as antisense DNAs and RNAs or chimeras thereof, miRNAs, and siRNAs that reduce FoxO1 expression. Based on known sequences of the targeted mRNA and the genes encoding FoxO1, therapeutic oligonucleotides can be engineered using methods known in the art. Different combinations of these therapeutic agents can be formulated for administration to a subject using methods known in the art.

Antisense-RNAs and anti-sense DNAs have been used therapeutically in mammals to treat various diseases. See for example Agrawal, S, and Zhao, Q. (1998) Curr. Opin. Chemical Biol. Vol. 2, 519-528; Agrawal, S. and Zhang, R. (1997) CIBA Found. Symp. Vol. 209, 60-78; and Zhao, Q, et al., (1998), Antisense Nucleic Acid Drug Dev. Vol 8, 451-458. An antisense PS-oligodeoxyribonucleotide for treatment of cytomegalovirus retinitis in AIDS patients is the first antisense oligodeoxyribonucleotide approved for human use in the US. Anderson, K. O., et al., (1996) Antimicrobiol. Agents Chemother. Vol. 40, 2004-2011, and U.S. Pat. No. 6,828,151 by Borchers, et al., entitled “Antisense modulation of hematopoietic cell protein tyrosine kinase expression,” describe methods for making and using antisense nucleic acids and their formulation. In an embodiment of the invention, the nucleic acid is conjugated to a phosphate group or other targeting ligand to facilitate uptake by osteoblasts. Details for making cDNAs, antisense DNAs, antisense RNAs, miRNAs, ribozymes, and small interfering RNAs are set forth in Karsenty, Gerard et al., U.S. Application Serial No. 20100190697.

The siRNA Oblimersen (Genasense^{R}) has been given to patients for up to six cycles of 7 days at a 3 mg/kg/day dose with no severe adverse effects. Oligonucleotides are relatively safe, and have been administered in amounts ranging from about 0.1 mg/kg to about 50 mg/kg. In an embodiment the oligonucleotides are delivered for example, intravenously. MOLECULAR THERAPY Vol. 13, No. 4, April 2006. Administration of the therapeutic agents or compositions in embodiments of the invention, may be accomplished using any of the conventionally accepted modes of administration, and doses will vary as described below.

The DNA, mRNA, and cDNA sequences of FoxO1 suitable for designing Fox01-specific inhibitory oligonucleotides are in the public domain and are Gene Bank Nos.:
Mus musculus FoxO1 cDNA: NM_{—}019739; DNA: NC_{—}000069
Homo sapiens FoxO1 cDNA: NM_{—}002015; DNA: NC_{—}000013

The oligonucleotides that may be used as agents herein are synthesized in vitro and do not include compositions of biological origin. Based on these known sequences of the targeted mRNAs and the genes encoding them, therapeutic oligonucleotides can be engineered using methods known in the art.

Antisense oligonucleotides can also inhibit mRNA translation into protein. In the case of antisense constructs, these single stranded deoxynucleic acids have a complementary sequence to that of the target protein mRNA and can bind to the mRNA by Watson-Crick base pairing. This binding either prevents translation of the target mRNA and/or triggers RNase H degradation of the mRNA transcripts. Consequently, antisense oligonucleotides have tremendous potential for specificity of action (i.e., down-regulation of a specific disease-related protein). To date, these compounds have shown promise in several in vitro and in vivo models, including models of inflammatory disease, cancer, and HIV (reviewed in Agrawal, Trends in Biotech. 14:376-387 (1996)). Antisense can also affect cellular activity by hybridizing specifically with chromosomal DNA. Advanced human clinical assessments of several antisense drugs are currently underway.

It is desirable to optimize the stability of the phosphodiester internucleotide linkage and minimize its susceptibility to exonucleases and endonucleases in serum. (Zelphati, O., et al., Antisense. Res. Dev. 3:323-338 (1993); and Thierry, A. R., et al., pp 147-161 in Gene Regulation: Biology of Antisense RNA and DNA (Eds. Erickson, R P and Izant, J G; Raven Press, NY (1992)).

Therapeutic nucleic acids being currently being developed do not employ the basic phosphodiester chemistry found in natural nucleic acids, because of these and other known problems. Modifications have been made at the internucleotide phosphodiester bridge (e.g., using phosphorothioate, methylphosphonate or phosphoramidate linkages), at the nucleotide base (e.g., 5-propynyl-pyrimidines), or at the sugar (e.g., 2′-modified sugars) (Uhlmann E., et al. Antisense: Chemical Modifications. Encyclopedia of Cancer, Vol. X., pp 64-81 Academic Press Inc. (1997)). Others have attempted to improve stability using 2′-5′ sugar linkages (see, e.g., U.S. Pat. No. 5,532,130).

Small interfering RNA (siRNA) has essentially replaced antisense and ribozymes as the next generation of targeted oligonucleotide drugs under development. SiRNAs are RNA duplexes normally 16-30 nucleotides long that can associate with a cytoplasmic multi-protein complex known as RNAi-induced (RISC). RI Silencing complex loaded with siRNA mediates the degradation of homologous mRNA transcripts; therefore siRNA can be designed to knock down protein expression with high specificity. Unlike other antisense technologies, siRNA functions through a natural mechanism evolved to control gene expression through non-coding RNA. This is generally considered to be the reason why their activity is more potent in vitro and in vivo than either antisense or ribozymes. A variety of RNAi reagents, including siRNAs targeting clinically relevant targets, are currently under pharmaceutical development, as described, e.g., in de Fougerolles, A. et al., Nature Reviews 6:443-453 (2007).

While the first described RNAi molecules were RNA: RNA hybrids comprising both an RNA sense and an RNA antisense strand, it has now been demonstrated that DNA sense: RNA antisense hybrids, RNA sense: DNA antisense hybrids, and DNA: DNA hybrids are capable of mediating RNAi (Lamberton, J. S, and Christian, A. T., (2003) Molecular Biotechnology 24: 111-119). Thus, the invention includes the use of RNAi molecules comprising any of these different types of double-stranded molecules. In addition, it is understood that RNAi molecules may be used and introduced to cells in a variety of forms.

Accordingly, as used herein, RNAi molecules encompasses any and all molecules capable of inducing an RNAi response in cells, including, but not limited to, double-stranded oligonucleotides comprising two separate strands, i.e. a sense strand and an antisense strand, e.g., small interfering RNA (siRNA); double-stranded oligonucleotide comprising two separate strands that are linked together by non-nucleotidyl linker; oligonucleotides comprising a hairpin loop of complementary sequences, which forms a double-stranded region, e.g., shRNAi molecules, and expression vectors that express one or more polynucleotides capable of forming a double-stranded polynucleotide alone or in combination with another polynucleotide.

A “single strand siRNA compound” as used herein, is a siRNA compound which is made up of a single molecule. It may include a duplexed region, formed by intra-strand pairing, e.g., it may be, or include, a hairpin or pan-handle structure. Single strand siRNA compounds may be antisense with regard to the target molecule.

A single strand siRNA compound may be sufficiently long that it can enter the RISC and participate in RISC mediated cleavage of a target mRNA. A single strand siRNA compound is typically at least 14, and in other embodiments at least 15, 20, 25, 29, 35, 40, or 50 nucleotides in length. In certain embodiments, it is less than 200, 100, or 60 nucleotides in length.

Hairpin siRNA compounds will have a duplex region equal to or at least 17, 18, 19, 29, 21, 22, 23, 24, or 25 nucleotide pairs. The duplex region will may be equal to or less than 200, 100, or 50, in length. In certain embodiments, ranges for the duplex region are 15-30, 17 to 23, 19 to 23, and 19 to 21 nucleotides pairs in length. The hairpin may have a single strand overhang or terminal unpaired region. In certain embodiments, the overhangs are 2-3 nucleotides in length. In some embodiments, the overhang is at the sense side of the hairpin and in some embodiments on the antisense side of the hairpin.

A “double stranded siRNA compound” as used herein, is a siRNA compound which includes more than one, and in some cases two, strands in which interchain hybridization can form a region of duplex structure.

The antisense strand of a double stranded siRNA compound may be equal to or at least, 14, 15, 16 17, 18, 19, 25, 29, 40, or 60 nucleotides in length. It may be equal to or less than 200, 100, or 50, nucleotides in length. Ranges may be 17 to 25, 19 to 23, and 19 to 21 nucleotides in length. As used herein, term “antisense strand” means the strand of a siRNA compound that is sufficiently complementary to a target molecule, e.g. a target mRNA.

The sense strand of a double stranded siRNA compound may be equal to or at least 14, 15, 16 17, 18, 19, 25, 29, 40, or 60 nucleotides in length. It may be equal to or less than 200, 100, or 50, nucleotides in length. Ranges may be 17 to 25, 19 to 23, and 19 to 21 nucleotides in length.

The double strand portion of a double stranded siRNA compound may be equal to or at least, 14, 15, 16 17, 18, 19, 20, 21, 22, 23, 24, 25, 29, 40, or 60 nucleotide pairs in length. It may be equal to or less than 200, 100, or 50, nucleotides pairs in length. Ranges may be 15-30, 17 to 23, 19 to 23, and 19 to 21 nucleotides pairs in length.

In many embodiments, the siRNA compound is sufficiently large that it can be cleaved by an endogenous molecule, e.g., by Dicer, to produce smaller siRNA compounds, e.g., siRNA agents. The sense and antisense strands may be chosen such that the double-stranded siRNA compound includes a single strand or unpaired region at one or both ends of the molecule. Thus, a double-stranded siRNA compound may contain sense and antisense strands, paired to contain an overhang, e.g., one or two 5′ or 3′ overhangs, or a 3′ overhang of 1-3 nucleotides. The overhangs can be the result of one strand being longer than the other, or the result of two strands of the same length being staggered. Some embodiments will have at least one 3′ overhang. In one embodiment, both ends of a siRNA molecule will have a 3′ overhang. In some embodiments, the overhang is 2 nucleotides.

In certain embodiments, the length for the duplexed region is between 15 and 30, or 18, 19, 20, 21, 22, and 23 nucleotides in length, e.g., in the ssiRNA compound range discussed above. ssiRNA compounds can resemble in length and structure the natural Dicer processed products from long dsiRNAs. Embodiments in which the two strands of the ssiRNA compound are linked, e.g., covalently linked are also included. Hairpin, or other single strand structures which provide the required double stranded region, and a 3′ overhang are also within the invention. The siRNA compounds described herein, including double-stranded siRNA compounds and single-stranded siRNA compounds can mediate silencing of a target RNA, e.g., mRNA, e.g., an mRNA transcript of a gene that encodes a protein. A gene may also be targeted.

In one embodiment, an siRNA compound is “sufficiently complementary” to a target RNA, e.g., a target mRNA, such that the siRNA compound silences production of protein encoded by the target mRNA. In another embodiment, the siRNA compound is “exactly complementary” to a target RNA, e.g., the target RNA and the siRNA compound anneal, for example to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, in certain embodiments, the siRNA compound specifically discriminates a single-nucleotide difference. In this case, the siRNA compound only mediates RNAi if exact complementary is found in the region (e.g., within 7 nucleotides of) the single-nucleotide difference.

### Pharmaceutical Formulations and Administration

The present invention encompasses pharmaceutical formulations of therapeutically effective amounts of Lcn-2 and at least one other active agent described herein, for administration to a subject in an amount sufficient to treat or prevent an enumerated disease or disorder.

A therapeutically effective amount of an active agent administered to treat or prevent type 1 or type 2 diabetes or metabolic syndrome in an animal is an amount that ameliorates one or more symptoms of the disease, typically by producing at least one effect selected from the group comprising increasing insulin express and/or secretion, increasing glucose tolerance, insulin sensitivity, glucose metabolism, weight loss, pancreatic β-cell proliferation, or glucose tolerance or by, decreasing fat mass, increasing serum adiponectin levels or decreasing serum resistin levels, which indicia can be measured or determined using standard methods known in the art.

A therapeutically effective amount of Lcn-2 or other active ingredient such as a protein or polypeptide (small molecule) for use in the present invention typically varies and can be an amount sufficient to achieve serum therapeutic agent levels typically of between about 0.5 nanograms per milliliter and about 100 micrograms per milliliter in the subject, or between about 0.5 nanograms per milliliter to about 15 micrograms per milliliter. Other preferred serum therapeutic agent levels include about 0.1 nanograms per milliliter to about 3 micrograms per milliliter, about 0.5 nanograms per milliliter to about 1 microgram per milliliter, about 1 nanogram per milliliter to about 750 nanograms per milliliter, about 5 nanograms per milliliter to about 500 nanograms per milliliter, and about 5 nanograms per milliliter to about 100 nanograms per milliliter.

Expressed as a daily dose, for Lcn-2, this amount can be between about 0.01 nanograms per kilogram body weight per day and about 40 milligrams per kilogram body weight per day, and between about 1-10 milligrams per kilogram body weight per day. Other preferred daily dosages include about 0.5 nanograms-40 milligrams per kilogram body weight per day, about 5 nanograms-5 milligrams per kilogram body weight per day, about 20 nanograms per-500 micrograms per kilogram body weight per day, and about 500 nanograms—100 micrograms per kilogram body weight per day. However, the skilled artisan will appreciate that certain factors may influence the dosage required to effectively treat a subject, including but not limited to the severity of the condition, previous treatments, the general health and/or age of the subject, and other disorders or diseases present.

Therapeutic amounts of inhibitory oligonucleotides that have been administered to humans varies, ranging from about 0.1 mg/kg to about 50 mg. Active agents to combine into pharmaceutical formulations with Lcn-2 include therapeutic oligonucleotides as described herein include the following compounds. The 2005 Physician's Desk Reference (PDR) describes administering an oral formulation or rosiglitazone in amounts of from about 8 mg/day to about 20 mg/day for treating diabete's (page 1442), and pioglitazone in amounts of from about 8 mg/day to about 45 mg/day (page 3185). Pioglitazone is preferred as it has fewer deleterious effects on blood lipids. Alpha-glucose inhibitors are exemplified by miglitol and acarbose. Dipeptidyl peptidase 4 (DPP4) inhibitors are exemplified by vildagliptin, sitagliptin and saxagliptin. Generally, there are six categories of insulins: rapid-acting, short-acting, intermediate acting, long acting, very long acting, and premixed. Insulin can be administered in children and adults subcutaneously 0.5 to 1 units/kg/day. Dosages may be adjusted according to advice of a health-care practitioner to achieve premeal and bedtime blood glucose levels of 80 to 140 mg/dL. Incretins are a type of gastrointestinal hormone that cause an increase in the amount of insulin released from the beta-cells of the islets of Langerhans after eating, even before blood glucose levels become elevated. Incretins are exemplified by glucagon-like peptide-1 (GLP-1) and Gastric inhibitory peptide (aka glucose-dependent Insulinotropic peptide or GIP). Metformin hydrochloride may be administered orally in adults as a tablet or oral solution at an initial dosage of 500-850 mg once daily in the morning.

Anticoagulants useful in the invention are exemplified by vitamin K antagonists, heparin and derivatives of heparin, and direct thrombin inhibitors. Vitamin K antagonists are exemplified by warfarin (also known under the brand names COUMADIN®, JANTOVEN®, MAREVAN®, and WARAN®), warfarin derivatives, acenocoumarol, phenprocoumon as well as phenindione. Heparin and derivatives of heparin are exemplified by low molecular weight heparin and fondaparinux. Direct thrombin inhibitors are exemplified by argatroban, lepirudin, bivalirudin and ximelagatran.

Vasodilators may be useful when co-administered with Lcn-2 in the present invention. Vasodilators are exemplified by adenosine, amyl nitrite and other nitrites, L-arginine, atrial natriuretic peptide (ANP), bradykinin, ethanol, endothelium-derived hyperpolarizing factor (EDHF), histamine, complement proteins C3a, C4a and C5a, niacin (nicotinic acid), nitric oxide, glyceryl trinitrate (commonly known as nitroglycerin), isosorbide mononitrate & isosorbide dinitrate, pentaerythritol tetranitrate (PETN), sodium nitroprusside, PDE5 inhibitors, sildenafil, tadalafil, vardenafil, platelet activating factor (PAF), prostacyclin (PGI.sub.2) as well as other prostaglandins, tetrahydrocannabinol (THC), theobromine, and papaverine.

Anticoagulants useful in the invention are exemplified by vitamin K antagonists, heparin and derivatives of heparin, and direct thrombin inhibitors. Vitamin K antagonists are exemplified by warfarin (also known under the brand names COUMADIN®, JANTOVEN®, MAREVAN®, and WARAN®), warfarin derivatives, acenocoumarol, phenprocoumon as well as phenindione. Heparin and derivatives of heparin are exemplified by low molecular weight heparin and fondaparinux. Direct thrombin inhibitors are exemplified by argatroban, lepirudin, bivalirudin and ximelagatran.

Vasodilators may be useful when co-administered with Lcn-2 in the present invention. Vasodilators are exemplified by adenosine, amyl nitrite and other nitrites, L-arginine, atrial natriuretic peptide (ANP), bradykinin, ethanol, endothelium-derived hyperpolarizing factor (EDHF), histamine, complement proteins C3a, C4a and C5a, niacin (nicotinic acid), nitric oxide, glyceryl trinitrate (commonly known as nitroglycerin), isosorbide mononitrate & isosorbide dinitrate, pentaerythritol tetranitrate (PETN), sodium nitroprusside, PDE5 inhibitors, sildenafil, tadalafil, vardenafil, platelet activating factor (PAF), prostacyclin (PGI.sub.2) as well as other prostaglandins, tetrahydrocannabinol (THC), theobromine, and papaverine.

Drugs used to treat the diabetic complication atherosclerosis are exemplified by statins, scilostazol, benzothiazepines, phenylalkylamines, dihydropyridines, epoprostenol, vitamin B3, and aspirin. Statins are further exemplified by atorvastatin, cerivastatin, fluvastatin, lovastatin, mevastatin, pitavastatin, pravastatin, rosuvastatin, and simvastatin. Benzothiazepines are exemplified by diltiazem. Phenylalkylamines are exemplified by verapamil. Dihydropyridines are exemplified by amlodipine, felodipine, isradipine, lacidipine, lercanidipine, nicardipine, nifedipine, nimodipine, nisoldipine, and nitrendipine.

Beta blockers are used to treat high blood pressure (hypertension), congestive heart failure (CHF), abnormal heart rhythms (arrhythmias), and chest pain (angina) associated with the enumerated disorders. Beta blockers are sometimes used in heart attack patients to prevent future heart attacks.

Brand Names and generic claims of beta blockers commonly used in the United States are: Betapace (sotalol), Blocadren (timolol), Brevibloc (esmolol), Cartrol (carteolol), Coreg (carvedilol), Corgard (nadolol), Inderal (propranolol), Inderal-LA (propranolol), Kerlone (betaxolol), Levatol (penbutolol), Lopressor (metoprolol), Normodyne (labetalol), Sectral (acebutolol), Tenormin (atenolol), Toprol-XL (metoprolol), Trandate (labetalol), Visken (pindolol), Zebeta (bisoprolol). Commonly Used Brand Names in Canada are: Apo-Atenolol (atenolol), Apo-Metoprolol (metoprolol), Apo-Propranolol (propranolol), Apo-Timol (timolol), Betaloc (metoprolol), Blocadren (timolol), Corgard (nadolol), Inderal (propranolol), Lopressor (metoprolol), Monitan (acebutolol), Novo-Atenol (atenolol), Novometoprol (metoprolol), Novo-Pindol (pindolol), Novo-Timol (timolol), Sectral (acebutolol), Sotacor (sotalol), Tenormin (atenolol), Trandate (labetalol), Trasicor (oxprenolol), Visken (pindolol).

To determine the efficacy of treatment, in some embodiments insulin sensitivity can be measured by the insulin tolerance test (ITT) or euglycemic hyperinsulinemic clamp. Glucose tolerance can be measured by glucose tolerance test (GTT). Insulin secretion can be measured by the glucose stimulated insulin secretion test (GSIS).

The traditional method of testing blood sugar involves pricking a finger with a lancet (a small, sharp needle), putting a drop of blood on a test strip and then placing the strip into a meter that displays a blood sugar level. These meters can also calculate an average blood sugar level over a period of time. Some meters also feature software kits that retrieve information from the meter and display graphs and charts of your past test results. In 1998, a laser to draw blood was approved by the U.S. Food and Drug Administration (FDA). Other methods of determining blood sugar levels include continuous glucose monitoring systems where a small plastic catheter (very small tube) is inserted just under the skin. It collects small amounts of fluid and measures the sugar content over 72 hours. In 2001, the FDA approved the GlucoWatch, a watch-like device that helps people with diabetes measure their blood sugar via tiny electric currents. The GlucoWatch draws small amounts of fluid from the skin and measures blood sugar levels three times per hour for up to 12 hours.

“Biological samples” include solid and body fluid samples. The biological samples of the present invention may include tissue, organs, cells, protein or membrane extracts of cells, blood or biological fluids such as blood, serum, ascites fluid or brain fluid (e.g., cerebrospinal fluid). Assays for detecting the levels of protein expression are well known to those of skill in the art. Such assays include, for example, antibody-based immunoassays. See Karsenty, Gerard et al., U.S. Application No. 20100190697.

“Pharmaceutically acceptable carrier” is intended to include any and all solvents, binders, diluents, disintegrants, lubricants, dispersion media, coatings, antibacterial and antifungal agents, isotonic and absorption delaying agents, and the like, compatible with pharmaceutical administration. The use of such media and agents for pharmaceutically active substances is well known in the art. As long as any conventional media or agent is compatible with the active compound, such media can be used in the compositions of the invention and supplementary active compounds or therapeutic agents can also be incorporated into the compositions. A pharmaceutical composition of the invention is formulated to be compatible with its intended route of administration. Examples of routes of administration include parenteral, e.g., intravenous, intradermal, intranasal, subcutaneous, oral, inhalation, transdermal (topical), transmucosal, and rectal administration.

The term “administer” is used in its broadest sense and includes any method of introducing the compositions of the present invention into a subject. An embodiment of the present invention includes producing Lcn-2 in vivo by transcription or translation of polynucleotides encoding Lcn-2 that have been exogenously introduced into a subject. Thus, polypeptides or nucleic acids produced in the subject from the exogenous compositions are encompassed in the term “administer.”

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylene diamine tetra acetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where the therapeutic agents are water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringability exists. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., Lcn-2 protein) in the required amount in an appropriate solvent with one or a combination of the ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. Depending on the specific conditions being treated, pharmaceutical compositions of the present invention for treatment of diabetes, obesity or the other elements of metabolic syndrome can be formulated and administered systemically or locally. Techniques for formulation and administration can be found in “Remington: The Science and Practice of Pharmacy” (20th edition, Gennaro (ed.) and Gennaro, Lippincott, Williams & Wilkins, 2000). For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL®, or corn starch; a lubricant such as magnesium stearate or STEROTES®; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. If appropriate, the compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to particular cells with, e.g., monoclonal antibodies) can also be used as pharmaceutically acceptable carriers. These can be prepared according to methods known to those skilled in the art, for example, as described in U.S. Pat. No. 4,522,811.

It is especially advantageous to formulate oral or parenteral compositions in unit dosage form for ease of administration and uniformity of dosage. “Unit dosage form” as used herein refers to physically discrete units suited as unitary dosages for the subject to be treated; each unit containing a predetermined quantity of active compound calculated to produce the desired therapeutic effect in association with the required pharmaceutical carrier. The specification for the unit dosage forms of the invention are dictated by and directly dependent on the unique characteristics of the active compound and the particular therapeutic effect to be achieved, and the limitations inherent in the art of compounding such an active compound for the treatment of individuals.

As previously noted, the agent may be administered continuously by pump or frequently during the day for extended periods of time. It will also be appreciated that the effective dosage may increase or decrease over the course of a particular treatment. Changes in dosage may result and become apparent from monitoring the level of Lcn-2 and/or insulin and/or monitoring glycemia control in a biological sample from the patient, preferably blood or serum. Glucose tolerance and insulin sensitivity can also be measured.

In an embodiment of the invention, the agent can be delivered by subcutaneous, long-term, automated drug delivery using an osmotic pump to infuse a desired dose of the agent for a desired time. Insulin pumps are widely available and are used by diabetics to automatically deliver insulin over extended periods of time. Such insulin pumps can be adapted to deliver the agent. The delivery rate of the agent to control glucose intolerance, diabetes types 1 or 2 can be readily adjusted through a large range to accommodate changing insulin requirements of an individual (e.g., basal rates and bolus doses). New pumps permit a periodic dosing manner, i.e., liquid is delivered in periodic discrete doses of a small fixed volume rather than in a continuous flow manner. The overall liquid delivery rate for the device is controlled and adjusted by controlling and adjusting the dosing period. The pump can be coupled with a continuous blood glucose monitoring device and remote unit, such as a system described in U.S. Pat. No. 6,560,471. In such an arrangement, the hand-held remote unit that controls the continuous blood glucose monitoring device could wirelessly communicate with and control both the blood glucose monitoring unit and the fluid delivery device delivering therapeutic agents of the present invention.

In other embodiments of the invention, a pharmaceutical formulation of the present invention can be a sustained release formulation such as a tablet. In some embodiments, the agent is continuously released from the controlled release formulation for up to between about 2-24 hours.

In certain embodiments, the methods comprise identifying a patient in need of treatment. Type 1 diabetes is usually diagnosed in children and young adults, and was previously known as juvenile diabetes. In type 1 diabetes, the body does not produce insulin. Conditions associated with type 1 diabetes include hyperglycemia, hypoglycemia, ketoacidosis and celiac disease. Type 2 diabetes is the most common form of diabetes in which either the body does not produce enough insulin or the cells ignore the insulin. Conditions associated with type 2 diabetes include hyperglycemia and hypoglycemia. Disorders associated with impaired energy metabolism include diabetes, glucose intolerance, decreased insulin sensitivity, decreased pancreatic beta-cell proliferation, decreased insulin secretion, weight gain, increased fat mass and decreased serum adiponectin.

### Screening Methods

A method is provided for testing an agent's effectiveness in increasing both Lcn-2 expression and secretion in osteoblasts and insulin expression or secretion in pancreatic beta cells, comprising: (a) co-culturing the osteoblasts and pancreatic β cells, (b) contacting the osteoblasts with a candidate agent, (c) determining whether the candidate agent significantly increases the level of both Lcn-2 expression or secretion in the osteoblasts and insulin expression or secretion above a respective control level measured in a control co-culture in which osteoblasts are not contacted with the candidate agent, and (d) if the candidate agent significantly increases both levels above the control level, then selecting the candidate agent as an agent that increases insulin expression or secretion in pancreatic beta cells. “Significantly increased” or “significantly higher” is at least about a 15% increase over control levels or pretreatment levels. This assay can be done other ways by studying osteoblasts alone, for example, to determine the agent's effect on Lcn-2 levels. A person of skill in the art knows how to vary this method.

A method is provided for determining the ability of a candidate agent to treat or prevent in an animal metabolic syndrome or a phenotype associated with metabolic syndrome that is selected from the group comprising predisposition to type 1 or 2 diabetes, glucose intolerance, decreased insulin production, decreased insulin sensitivity, decreased glucose tolerance, atherosclerosis and increased fat mass, comprising: (a) providing a test animal and a control animal, (b) administering the candidate agent to the test animal, (c) comparing the level of Lcn-2 in the test animal to the level of Lcn-2 in the control animal, and (d) selecting the candidate agent if the level of Lcn-2 is significantly higher in the test animal than in the control animal. “Significantly increased” or “significantly higher” is at least about a 15% increase over control levels or pretreatment levels. In a specific embodiment of the invention the level of Lcn-2 is measured in osteoblasts. The candidate agent may be bound to a phosphate group that facilitates its uptake by osteoblasts.

The term “agent” or “exogeneous compound” as used herein includes any molecule, e.g., protein, oligopeptide, small organic molecule, polysaccharide, polynucleotide, lipid, etc., or mixtures thereof, with the capability of directly or indirectly altering the bioactivity of Lcn-2. Generally a plurality of assay mixtures is run in parallel with different agent concentrations to obtain a differential response to the various concentrations. Typically, one of these concentrations serves as a negative control, i.e., at zero concentration or below the level of detection.

Known and novel pharmacological agents identified in screens may be further subjected to directed or random chemical modifications, such as acylation, alkylation, esterification, amidification to produce structural analogs. The agent may be a protein. By “protein” in this context is meant at least two covalently attached amino acids, which includes proteins, polypeptides, oligopeptides and peptides. The protein may be made up of naturally occurring amino acids and peptide bounds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acids” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations

The agent may be a naturally occurring protein or fragment or variant of a naturally occurring protein. Thus, for example, cellular extracts containing proteins, or random or directed digests of proteinaceous cellular extracts, may be used. In this way, libraries of prokaryotic and eukaryotic proteins may be made for screening. Libraries of bacterial, fungal, viral, and mammalian proteins, with the latter being preferred, and human proteins being especially preferred may be used. Agents may be peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized agent bioactive proteinaceous agents. Further variations and details are set forth in Karsenty US application 20100190697.

### Biologically Active Fragments or Variants of Lcn-2

As used herein, Lcn-2 includes biologically active fragments or variants. “Biologically active” means increasing at least one effect selected from the group comprising increasing pancreatic beta-cell proliferation, increasing insulin expression and secretion, increasing insulin sensitivity, increasing glucose tolerance, decreasing weight gain, decreasing fat mass, increasing weight loss, increasing serum adiponectin levels or decreasing resistin levels and increasing muscle mass or myogenesis. Fragments can be discrete (not fused to other amino acids or polypeptides) or can be within a larger polypeptide. Further, several fragments can be comprised within a single larger polypeptide. In one embodiment, a fragment designed for expression in a host can have heterologous pre- and pro-polypeptide regions fused to the amino terminus of the Lcn-2 fragment and/or an additional region fused to the carboxyl terminus of the fragment. It will be understood that a biologically active fragment or variant of human Lcn-2 may contain a different number of amino acids than native human Lcn-2. Accordingly, the position number of the amino acid residues corresponding to certain positions of mature human Lcn-2 may differ in the fragment or variant. One skilled in the art would easily recognize such corresponding positions from a comparison of the amino acid sequence of the fragment or variant with the amino acid sequence of mature human Lcn-2.

The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

“Lcn-2 Variants” can be naturally-occurring or can be made by recombinant means, or chemical synthesis, to provide useful and novel characteristics for Lcn-2. For example, the variant Lcn-2 polypeptides may have reduced immunogenicity, increased serum half-life, increased bioavailability and/or increased potency. “Variants” refers to Lcn-2 peptides that contain modifications in their amino acid sequences such as one or more amino acid substitutions, additions, deletions and/or insertions but that are still biologically active. In some instances, the antigenic and/or immunogenic properties of the variants are not substantially altered, relative to the corresponding peptide from which the variant was derived. Such modifications may be readily introduced using standard mutagenesis techniques, such as oligonucleotide directed site-specific mutagenesis as taught, for example, by Adelman et al. (DNA, 2:183, 1983) or by chemical synthesis. Variants and fragments are not mutually exclusive terms. Fragments also include peptides that may contain one or more amino acid substitutions, additions, deletions and/or insertions such that the fragments are still biologically active. Fully functional variants typically contain only conservative variation or variation in non-critical residues or in non-critical regions. Functional variants can also contain substitutions of similar amino acids, which results in no change, or an insignificant change, in function. Alternatively, such substitutions may positively or negatively affect function to some degree. The activity of such functional Len-2 variants can be determined using assays such as those described herein.

Some variants are also derivatives of the Lcn-2 and Lcn-2 fragments. Derivatization is a technique used in chemistry which transforms a chemical compound into a product of similar chemical structure, called derivative. Generally, a specific functional group of the compound participates in the derivatization reaction and transforms the educt to a derivate of deviating reactivity, solubility, boiling point, melting point, aggregate state, functional activity, or chemical composition. Resulting new chemical properties can be used for quantification or separation of the educt or can be used to optimize the compound as a therapeutic agent. The well-known techniques for derivatization can be applied to the above-described Lcn-2 and Lcn-2 fragments. Thus, derivatives of the Lcn-2 and Lcn-2 fragments described above will contain amino acids that have been chemically modified in some way so that they differ from the natural amino acids.

Provided also are Lcn-2 mimetics. “Mimetic” refers to a synthetic chemical compound that has substantially the same structural and functional characteristics of a naturally or non-naturally occurring polypeptide, and includes, for instance, polypeptide- and polynucleotide-like polymers having modified backbones, side chains, and/or bases. Peptide mimetics are commonly used in the pharmaceutical industry as non-peptide drugs with properties analogous to those of the template peptide. Generally, mimetics are structurally similar (i.e., have the same shape) to a paradigm polypeptide that has a biological or pharmacological activity, but one or more polypeptide linkages are replaced. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity.

By way of example, example, Cho et al., 1993, Science 261:1303-5 discloses an “unnatural biopolymer” comprising chiral aminocarbonate monomers substituted with a variety of side chains, synthesis of a library of such polymers, and screening for binding affinity to a monoclonal antibody. Similarly, Cho et al, 1998, J. Am. Chem. Soc. discloses libraries of linear and cyclic oligocarbamate libraries and screening for binding to the integrin GPIIb/IIIa. Simon et al., Proc. Natl. Acad. Sci. 89:9367-71 discloses a polymer comprising N-substituted glycines (“peptoids”) with diverse side chains. Schumacher et al, 1996, Science 271:1854-7 discloses D-peptide ligands specific for Src homology domain 3 (SH3 domain) by screening phage libraries of L-peptides against a proteins (SH3) synthesized with D-amino acids and then synthesizing a selected L-peptide using D-amino acids. Brody et al., 1999, Mol. Diagn. 4: 381-8 describes generation and screening of hundreds to thousands of aptamers.

Lcn-2 molecules that fall within the scope of the invention include proteins substantially homologous to human Lcn-2 including proteins derived from another organism, i.e., an ortholog.

Orthologs of Lcn2

| Species | Human | Mouse |
|---|---|---|
| Entrez | 3934 | 16819 |
| Ensembl | ENSG00000148346 | ENSMUSG00000026822 |
| UniProt | P80188 | Q3UE34 |
| RefSeq | NM_005564 | NM_008491 |
| (mRNA) | | |
| RefSeq | NP_005555 | NP_032517 |
| (protein) | | |
| Location | Chr 9: | Chr 2: |
| (UCSC) | 129.95-129.96 Mb | 32.21-32.21 Mb |

As used herein, two proteins are substantially homologous, or identical, when their amino acid sequences are at least about 70-75%, typically at least about 80-85%, and most typically at least about 90-95%, 97%, 98% or 99% or more homologous. “Homology” between two amino acid sequences or nucleic acid sequences can be determined by using the algorithms disclosed herein. These algorithms can also be used to determine percent identity between two amino acid sequences or nucleic acid sequences. Methods for determining sequence homology are well known.

The invention also encompasses polypeptides having a lower degree of identity but which have sufficient similarity so as to perform one or more of the same functions performed by Lcn-2. Similarity is determined by considering conserved amino acid substitutions. Such substitutions are those that substitute a given amino acid in a polypeptide by another amino acid of like characteristics. Conservative substitutions are likely to be phenotypically silent. Guidance concerning which amino acid changes are likely to be phenotypically silent is found in Bowie et al., Science 247:1306-1310 (1990).

Examples of conservative substitutions are the replacements, one for another, among the hydrophobic amino acids Ala, Val, Leu, and Ile; interchange of the hydroxyl residues Ser and Thr; exchange of the acidic residues Asp and Glu; substitution between the amide residues Asn and Gln; exchange of the basic residues Lys, His and Arg; replacements among the aromatic residues Phe, Trp and Tyr; exchange of the polar residues Gln and Asn; and exchange of the small residues Ala, Ser, Thr, Met, and Gly.

The comparison of sequences and determination of percent identity and homology between two Lcn-2 polypeptides can be accomplished using a mathematical algorithm. For example, Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H G., eds., Humana Press, New Jersey, 1994; Sequence Analysis in Molecular Biology, van Heinje, G., Academic Press, 1987; and Sequence Analysis Primer, Gribskov, M. and Devereux, J., eds., M Stockton Press, New York, 1991. A non-limiting example of such a mathematical algorithm is described in Karlin et al. (1993) Proc. Natl. Acad. Sci. USA 90: 5873-5877. The percent identity or homology between two Lcn-2 amino acid sequences may be determined using the Needleman et al. (1970) (J. Mol. Biol. 48:444-453) algorithm. Another non-limiting example of a mathematical algorithm that may be utilized for the comparison of sequences is the algorithm of Myers and Miller, CABIOS (1989).

A substantially homologous Lcn-2, according to the present invention, may also be a polypeptide encoded by a nucleic acid sequence capable of hybridizing to the human Lcn-2 nucleic acid sequence under highly stringent conditions, e.g., hybridization to filter-bound DNA in 0.5 M NaHPO₄, 7% sodium dodecyl sulfate (SDS), 1 mM EDTA at 65 degrees Celsius, and washing in 0.1.times SSC/0.1% SDS at 68 degrees Celsius. (Ausubel F. M. et al. eds., 1989, Current Protocols in Molecular Biology, Vol. I, Green Publishing Associates, Inc., and John Wiley & sons, Inc., New York, at p. 2.10.3) and encoding a functionally equivalent gene product; or under less stringent conditions, such as moderately stringent conditions, e.g., washing in 0.2.times, SSC/0.1% SDS at 42 degrees Celsius (Ausubel et al., 1989 supra), yet which still encodes a biologically active Lcn-2.

A substantially homologous Lcn-2, according to the present invention, may also be a polypeptide encoded by a nucleic acid sequence capable of hybridizing to a sequence having at least 70-75%, typically at least about 80-85%, and most typically at least about 90-95%, 97%, 98% or 99% identity to the human Lcn-2 nucleic acid sequence, under stringent conditions, e.g., hybridization to filter-bound DNA in 0.5 M NaHPO₄, 7% sodium dodecyl sulfate (SDS), 1 mM EDTA at 65 degrees Celsius, and washing in 0.1.times.SSC/0.1% SDS at 68 degrees Celsius. (Ausubel F. M. et al., eds., 1989, Current Protocols in Molecular Biology, Vol. I, Green Publishing Associates, Inc., and John Wiley & sons, Inc., New York, at p. 2.10.3) and encoding a functionally equivalent gene product; or under less stringent conditions, such as moderately stringent conditions, e.g., washing in 0.2×SSC/0.1% SDS at 42 degrees Celsius. (Ausubel et al., 1989 supra), yet which still encodes a biologically active Lcn-2.

Peptides corresponding to fusion proteins in which full length Lcn-2, mature Lcn-2, or an Lcn-2 fragment or variant is fused to an unrelated protein or polypeptide are also within the scope of the invention and can be designed on the basis of the Lcn-2 nucleotide and amino acid sequences disclosed herein using routine methods known in the art. The Gene Bank Accession number for the mouse Lcn-2 gene (mRNA) is gene bank NM008491, the mouse cDNA is gene bank NM0084991.1, the mouse amino acid sequence/protein sequence is NP032517; the human gene (mRNA) is gene bank NM005564, and the amino acid/protein sequence is gene bank NP005555.

Such fusion proteins include fusions to an enzyme, fluorescent protein, or luminescent protein which provides a marker function. In a preferred embodiment of the invention, Lcn-2 is fused to a targeting the pancreas and the inhibitory oligonucleotide targeting FoxO1 is fused to a polypeptide targeting osteoblasts. For example, Lcn-2 polypeptide sequences may be fused to a ligand molecule capable of targeting the fusion protein to a cell expressing the Lcn2 receptor or to pancreatic β cells to enhance insulin expression and secretion. Lcn-2 can also be made as part of a chimeric protein for drug screening or use in making recombinant protein. These comprise an Lcn-2 peptide sequence operatively linked to a heterologous peptide having an amino acid sequence not substantially homologous to the Lcn-2. “Operatively linked” in this context indicates that the Lcn-2 peptide and the heterologous peptide are fused in-frame. The heterologous peptide can be fused to the N-terminus or C-terminus of Lcn-2 or can be internally located. In one embodiment, the fusion protein does not affect Lcn-2 function. For example, the fusion protein can be a GST-fusion protein in which the Lcn-2 sequences are fused to the N- or C-terminus of the GST sequences. Other types of fusion proteins include, but are not limited to, enzymatic fusion proteins, for example beta-galactosidase fusions, yeast two-hybrid GAL-4 fusions, poly-His fusions and Ig fusions. Such fusion proteins, particularly poly-His fusions, can facilitate the purification of recombinant Lcn-2. In certain host cells (e.g., mammalian host cells), expression and/or secretion of a protein can be increased by using a heterologous signal sequence. Therefore, the fusion protein may contain a heterologous signal sequence at its N-terminus.

EP-A 0 464 533 discloses fusion proteins comprising various portions of immunoglobulin constant regions (Fc regions) using known methods. (Bennett et al. (1995) J. Mol. Recog. 8: 52-58 (1995) and Johanson et al. J. Biol. Chem. 270:9459-9471). Thus, various embodiments of this invention also utilize soluble fusion proteins containing an Lcn-2 polypeptide and various portions of the constant regions of heavy or light chains of immunoglobulins of various subclasses (e.g., IgG, IgM, IgA, IgE, IgB).

A chimeric or fusion protein can be produced by standard recombinant DNA techniques.

Polypeptides often contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art. Common modifications that occur naturally in polypeptides are described below.

Lcn-2 polypeptides of the present invention also encompass derivatives which contain a substituted amino acid residue that is not one encoded by the genetic code, in which a substituent group is included, in which the mature polypeptide is fused with another compound, such as a compound to increase the half-life of the polypeptide (for example, polyethylene glycol), or in which the additional amino acids are fused to the Lcn-2 polypeptide, such as a leader or secretory sequence or a sequence for purification of the Lcn-2 polypeptide or a pro-protein sequence.

Lcn-2 can be modified according to known methods in medicinal chemistry to increase its stability, half-life, uptake or efficacy. Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Acylation of the N-terminal amino group can be accomplished using a hydrophilic compound, such as hydroorotic acid or the like, or by reaction with a suitable isocyanate, such as methylisocyanate or isopropylisocyanate, to create a urea moiety at the N-terminus. Other agents can also be N-terminally linked that will increase the duration of action of the Lcn-2 derivative as known in this art.

Reductive amination is the process by which ammonia is condensed with aldehydes or ketones to form imines which are subsequently reduced to amines. Reductive amination is a useful method for conjugating Lcn-2 and its fragments or variants to PEG. Covalent linkage of poly(ethylene glycol) (PEG) to Lcn-2 and its fragments and variants may result in conjugates with increased water solubility, altered bioavailability, pharmacokinetics, immunogenic properties, and biological activities. See, e.g., Bentley et al., J. Pharm. Sci. 1998 November; 87(11):1446-9.

Several particularly common modifications that may be applied to Lcn-2 and its fragments and variants such as glycosylation, lipid attachment, sulfation, hydroxylation and ADP-ribosylation are described in most basic texts, such as Proteins—Structure and Molecular Properties, 2nd ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al. (1990) Meth. Enzymol. 182: 626-646) and Rattan et al. (1992) Ann. NY: Acad. Sci. 663: 48-62.

Modifications can occur anywhere in the Lcn-2 and its fragments and variants, including the peptide backbone, the amino acid side-chains and the amino or carboxyl termini. Blockage of the amino or carboxyl group in a polypeptide, or both, by a covalent modification, is common in naturally-occurring and synthetic polypeptides and may be applied to the Lcn-2 or its fragments and variants of the present invention. For instance, the amino terminal residue of polypeptides made in E. coli, prior to proteolytic processing, almost invariably will be N-formylmethionine. Thus, Lcn-2 and its fragments and variants with N-formylmethionine as the amino terminal residue are within the scope of the present invention. A brief description of various protein modifications that come within the scope of this invention are described in Karsenty, Gerard et al., U.S. Application No. 20100190697.

### Recombinant Lcn-2

To practice the methods of the invention, it may be desirable to recombinantly express the Lcn-2 protein. The cDNA sequence and deduced amino acid sequence of human Lcn-2 are available from Gene Bank. The human gene (mRNA) is gene bank NM005564, and the amino acid/protein sequence is gene bank NP005555.

These methods include, for example, in vitro recombinant DNA techniques, synthetic techniques, and in vivo genetic recombination. (See, e.g., the techniques described in Sambrook et al., 1989, supra, and Ausubel et al., 1989, supra).

A variety of host-expression vector systems may be utilized to express the Lcn-2 nucleotide sequences. In a preferred embodiment, the Lcn-2 peptide or polypeptide is secreted and may be recovered from the culture media. Appropriate expression systems can be chosen to ensure that the correct modification, processing and subcellular localization of the Lcn-2 protein occurs. To this end, bacterial host cells are preferred for expression of Lcn-2; as such cells are unable to carboxylate Lcn-2. Further details for making recombinant Lcn-2 are set forth in Karsenty, Gerard et al., U.S. Application No. 20100190697.

The invention has been described with reference to specific embodiments. It will, however, be evident that various modifications and changes may be made thereto without departing from the broader spirit and scope of the invention. The specification and drawings are, accordingly, to be regarded in an illustrative rather than a restrictive sense. The invention is illustrated herein by the experiments described above and by the following examples, which should not be construed as limiting. Although specific terms are employed, they are used as in the art unless otherwise indicated.

### EXAMPLES

### Materials and Methods

Chemical Reagents

All chemical reagents were obtained from SIGMA.

Animals

All procedures involving animals were approved by the Institutional Animal Care and Use Committee at Columbia University. Mice were maintained under appropriate barrier conditions in a 12 hour light-dark cycle and received food and water ad libitum. The animals that were used for this study were C57BL6 mice including FOXO1 knockout and wild type mice. All studies were performed on the different genotypes with littermates as controls.

INS-1 Pancreatic Cells

INS-1 pancreatic cells were provided by Dr. Karsenty's laboratory at Columbia University. The cells were plated in RPMI, 10% FBS supplemented with 1 mM sodium pyruvate, 10 mM Hepes and 50 uM b-mercaptoethanol. Twenty four hours later, cells were washed and starved for 4 hours in RPMI, 0.5% FBS supplemented with 1 mM sodium pyruvate, 10 mM Hepes and 50 uM b-mercaptoethanol followed by treatment with various concentrations of lipocalin or vehicle for 4 hours.

Gene Expression Analyses

All gene expression analyses were performed using real time PCR. DNAse I-treated total RNA was converted to cDNA with the SuperScript III kit (Invitrogen). Real-time PCR were performed using the Taq SYBR Green Supermix with ROX (Biorad) on an MX3000 instrument (Stratagene); beta-actin amplification was used as an internal reference for each sample. All primers were from SuperArray. Gene expression was analyzed by quantitative real-time PCR. Total RNA was isolated from tissues or cultured osteoblastic cells using TRIZOL reagent. Total RNA (2 μg) was reversed transcribed at 42° C. with SuperScript II (Invitrogen). Quantitative real-time PCR was performed using the SYBR Green Master Mix (Bio-Rad). The efficiency of PCR application for each gene was calculated with the standard curve method (E=10^{-(1/log CURVE SLOPE)}). Relative mRNA abundance for each gene measured was calculated using the inverse of PCR efficiency raised to the power ΔCt (gene abundance=1/E^{ΔCt}, where ΔCt=Ct sample-Ct of reference gene) as previously reported (Nolan et al., 2006). β-actin has been used as reference gene.

Statistical Analyses

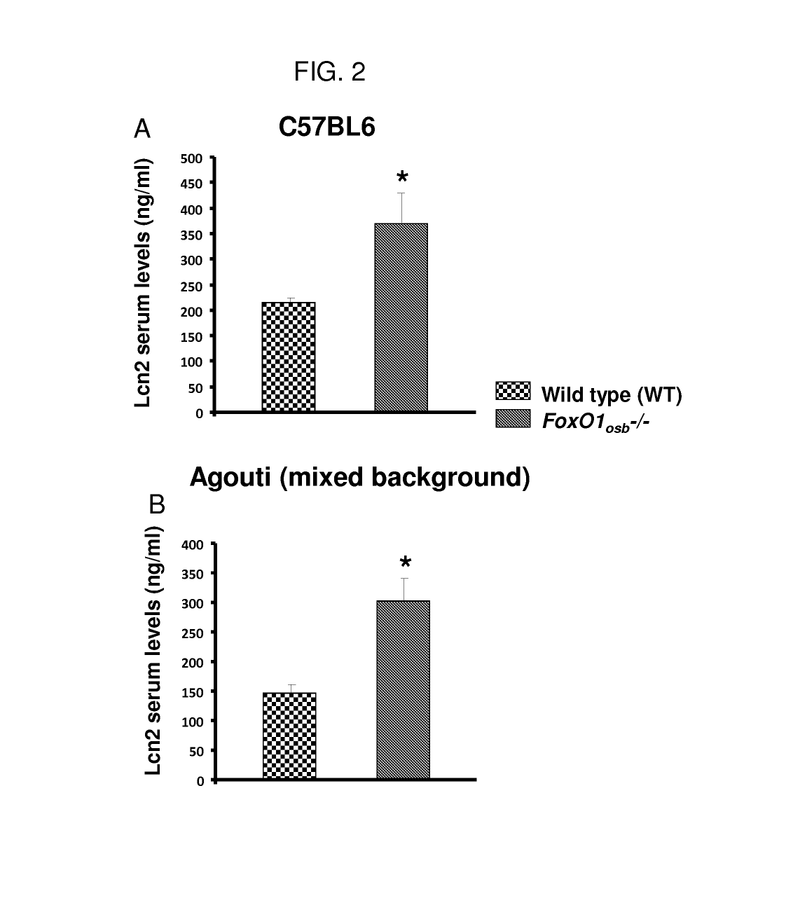

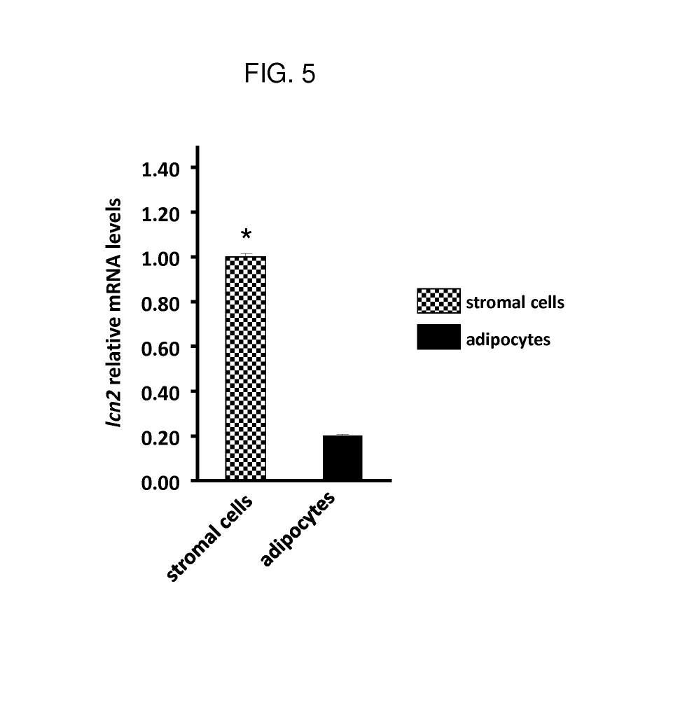

Results are given as means.+−.standard deviations except in FIGS. 2(B) and 5(F) where means.+−.standard errors of the mean are shown. Statistical analyses were performed using unpaired, two-tailed Student's t or ANOVA tests followed by post hoc tests. A p value <0.05 was considered significant and is indicated by a star in all figures unless otherwise indicated.

Method for Making Recombinant Lcn-2

Mouse recombinant Lcn-2 was prepared from murine myeloma cells and was purchased from R&Systems, Inc. (Mineapolis, Minn.), cat#1857-LC. It can also be prepared by expressing it in bacteria and purifying it. For the construction of the bacterial vector expressing lipocalin-2 fused with GST, pGEX lipocalin-2 83-625, the cDNA encoding mature mouse lipocalin-2 was subcloned into the BamHI/NotI sites of the pGEX-4T-3.vector (GE Healthcare). The GST-lipocalin-2 fusion protein was expressed in E. coli strain BL21pLys and purified on glutathione-Sepharose according to standard procedures. After extensive washes, lipocalin-2 was cleaved out from the GST moiety using thrombin. A HiTrap Benzamidine column is subsequently used to deplete the thrombin from the preparation. Purity is assessed by SDS-PAGE stained with Coomassie blue.

Comparative Microarray Analysis of FoxO1 Deficient Osteoblasts

Comparative microarray analysis of FoxO1 deficient osteoblasts was performed using a gene array experiment in osteoblasts derived from wild type mice and mice with osteoblast-specific inactivation of FoxO1. (FoxO1_{osb}−/−). For this purpose calvaria from 3 wild type and 3 FoxO1_{osb}−/− mice were removed and used to isolate osteoblasts as follows. Calvariae isolated from 3 day-old pups were subjected to four sequential, 30-minute long digestions in 1.5 U/ml collagenase-P (Roche) at 37° C. with gentle shaking. Cell fractions were collected, pooled and resuspended in Dulbecco's modified Eagle's medium (DMEM, Life Technologies) containing 10% FBS (Hyclone) and centrifuged. Cells were resuspended in DMEM containing 10% FBS. Total RNA was isolated using the RNeasy Mini kit (Quiagen). 5 μg of total RNA was used for the subsequent one cycle enzymatic synthesis of cRNA (Affymetrix, USA). Hybridization, washing/staining and scanning of Affymetrix Mouse genome 430 2.0 GeneChip Array were performed in triplicate according to the instructions provided by Affymetrix. Further analysis of gene expression was performed using Data Mining Tools (Affymetrix) and Partek genomatic software.

Details for Making Lcn-2 Knockout Mice

For the generation of the Lcn-2 knockout mice, embryonic stem (ES) cells targeted with the genomic clone of inactivated Lcn-2 were obtained from KOMP. KOPM is a mouse repository which carries constructs, ES cells or mice with inactivated alleles of several genes. Regeneron had targeted the Lcn-2 KO ES cells and provided them to KOMP as part of the knocking out the entire genome project

The floxed Lcn-2 construct for cell specific inactivation of Lcn-2 was also made by KOMP. The targeting vector harbors loxP sites within introns 2 and 6 designed to delete a 1.9 kb genomic fragment containing Lcn-2 exons 3-6. The neomycin resistance gene flanked by two FRT sites and driven by the human beta actin promoter was used for positive selection. The diphtheria toxin A gene (DTA) driven by the PGK promoter is incorporated into the 3′ end of the vector allowing for negative selection. The targeting vector was linearized with AsiSI and electroporated into 129/B6 ES cells. Homologous recombinants were identified by Southern blot with 5′ and 3′ PCR generated flanking probes and verified by PCR using primers specific for neo or the Lcn-2 intron 6 sequence-3′ loxP site. Mice are now being generated from the positive ES cells.

C2C12 Myocyte Experiments

C2C12 skeletal muscle cells were plated in DMEM (25 mM glucose) 10% FBS. Twenty four hours later, cells were washed and starved for 4 h in DMEM, 0.5% FBS and treated with various concentrations of recombinant lipocalin or vehicle for 4 h.

The Osteoblast-Deficient Mouse Model

Inducible ablation of osteoblasts was achieved by cross-breeding transgenic mice expressing a tamoxifen-regulated Cre under the control of the human osteocalcin promoter with mice in which an inactive form of the diphtheria toxin A chain (DTA) has been introduced into the ubiquitously expressed ROSA26 locus (DTA^{fl/fl}). The OCN promoter directs strong osteoblast-specific expression of genes in transgenic mice. In the DTA mice, DTA expression is prevented by the presence of a DNA STOP sequence which terminates transcription flanked by two loxP sites. When mice expressing both OCN-Cre and DTA^{fl/fl} are treated with tamoxifen treatment, the Cre recombinase is activated, and the stop cassette removed from the DTA locus, thereby inducing the expression of DTA in osteoblasts and killing them. Using these mouse models we have been able to ablate 50% of osteoblasts.

Mice

The ROSA26-lacZ^{fl/fl} mice were obtained from Jackson Laboratories (GtROSA26^{tm1Sho})⁶. The DTA mice were generated as follows. A Cre-regulated diphtheria toxin A chain (DTA) mini-gene was engineered into the Gt(ROSA)26Sor locus by targeting a ‘foxed STOP’ cassette (3′SS-LoxP-EM7-neo-pgkpolyA-tpA-LoxP) followed by an cDNA encoding for dta-ires-eGFP-βglpA into the XbaI site of the Gt(ROSA)26Sor locus (coordinates 113026025 to 113026030 on Chromosome 6, Ensembl release 60—November 2010). 3′ SS is a 3′ splice region consensus region having a sequence as set forth in Yoshikawa, Y., et al.¹³, EM7 is a prokaryotic promoter, neo is a neomycin phosphotransferase ORF⁷, pgkpA is a polyadenylation region derived from the phosphoglycerate kinase gene⁸, tpA is a polyadenylation region comprised of 3 tandem copies of an SV40-derived polyadenylation region⁶, DTA is an ORF encoding for diphtheria toxin A⁹, IRES is an internal ribosome entry site, eGFP is an enhanced Green Fluorescent Protein, and βglpA is a polyadenylation region derived from the rabbit beta globin gene. The foxed STOP cassette is identical to that utilized previously (Soriano, 1999), except that neo is lacking a mammalian promoter. This engineering modality renders its expression is dependent on integration into a transcriptionally active locus, and can thereby ‘facilitate’ targeting by reducing the number of non-productive integrations. Targeting is guided to the Gt(ROSA)26Sor locus by flanking this cassette and DTA-ires-eGFP-βglpA module with homology arms comprised of ˜2.4 and ˜2.8 kb 5′ and 3′ of the XbaI site. Targeting was performed into CJ7 ES cells as described¹⁰; 6 out of 40 colonies screened were correctly targeted. Of these, VG2128A-H4 and VG2182B-G5 were microinjected to generate mice. Mice harboring this targeted integration (Gt(ROSA)26Sor^{floxed-STOP}^{_{—}}^{dia/+} are termed DTAfl/fl and were physiologically normal, indicating that no DTA protein was expressed prior to removal of the floxed STOP region. The OCN-Cre-ER^{T2} transgene was constructed by fusing the human osteocalcin promoter and a cDNA encoding Cre-ER^{T2}. The plasmid pKB-Cre-ER^{T2 11} was cleaved with NotI and KpnI to isolate the Cre-ER^{T2} cDNA. The Cre-ER^{T2} fragment was then subcloned into pOC, which contains 3,900 bp of the human osteocalcin promoter and the second intron of rabbit β-globin on a pBluescript SK(−) backbone¹², to create pOC-Cre-ER^{T2}. The insert of this plasmid (OC-Cre-ER^{T2}) was excised and microinjected into fertilized eggs (FVB-N mouse strain). The wild-type and DTA^{flox} alleles were detected using PCR with primers having sequences for wild-type and for the DTA^{flox} allele as set forth in Yoshikawa, Y., et al.¹³ Genotyping was performed at 3 weeks of age by PCR analysis of genomic DNA. In all experiments data presented were obtained from male animals.

INS-1 Beta-Cell Line

INS-1 is the β-cell line that was used for the treatments. It is the best among other isolated cell lines in having insulin content (both Ins1 and Ins2) closer to that of normal islets and the ability to secrete insulin in response to glucose concentrations, in the physiological range. However, even the best rodent cell lines are imperfect. INS-1 cells generally show a 2- to 6-fold increase in insulin secretion in response to glucose which is less than the up to 15-fold responses achievable with freshly isolated primary islets. The highest levels of Ins1 and Ins2 gene expression were achieved with recombinant Lcn-2 treatment.

Mice, rat, Xenopus and some fishes, have two non-allelic Insulin genes, Ins1 and Ins2 that encode two very similar proinsulin proteins. Wentworth B M, et al. Characterization of the two nonallelic genes encoding mouse preproinsulin. J Mol Evol 23:305-312, 1986. These genes are both functional and the two proteins are synthesized in the pancreas in a 1:2 ratio for Ins1 and Ins2, respectively. The Ins1 gene arose by retrotransposition of a partially processed Ins2 transcript and is highly similar to it but lacks the second intron. Ins1 maps to the telomeric region of the mouse chromosome 19. The single Ins gene found in human, pig, chicken and other species has a structure similar to that of the mouse Ins2 gene, i.e. two introns and is considered as the ancestral Ins gene. The ancestral Ins gene is located in an evolutionary conserved imprinted chromosome domain, on the chromosome 7 in mouse and 11p15 in human.

Single homozygous null mouse mutants for Ins1 or Ins2 [where they deleted each one of the two genes] were not diabetic, and they had normal plasma insulin levels and normal glucose tolerance tests. Both genes needed to be deleted to have a diabetic phenotype, therefore the two genes appear to be able to compensate for one another. See Leroux L., et al., Compensatory responses in mice carrying a null mutation for Ins1 or Ins2, Diabetes. 2001 February; 50 Suppl 1:S150-3.

Metabolic Studies

Intraperitoneal glucose tolerance tests were performed by administering 2 g of glucose per kg of body weight (BW) intraperitoneally (IP) after an overnight fast. For parallel experiments of intraperitoneal glucose tolerance test (IPGTT) and oral glucose tolerance test (OGTT), 1.5 g of glucose were administered per kg of body weight (BW) either intraperitoneally (IP) or orally by gavage (O) after an overnight fast (22;23). Blood glucose was monitored using blood glucose strips (Diabetes association) and the Accu-Check glucometer. For glucose stimulated insulin secretion test (GSIS), 3 g/kg BW of glucose was injected IP after an overnight fast; plasma was collected from tails using heparinized microcapillaries and insulin measured at 0, 2, 5, 15 and 30 minutes. For insulin tolerance test (ITT) mice were fasted from 4-6 hours, injected IP with insulin (0.5 U/kg BW) and blood glucose levels were measured at indicated times. ITT data are presented as percentage of initial blood glucose concentration.

Energy Balance

Energy expenditure was measured by indirect calorimetric method using a six-chamber Oxymax system (Columbus Instruments, Ohio). Mice were acclimatized for 24 hr before measurements every 14 min for 48 hr. Body composition was measured with NMR (Bruker Optics). For food intake studies, mice were individually housed in metabolic cages (Nalgene, Rochester, N.Y.) and fed ad libitum. Food consumption amount was determined by weighing the powdered chow before and after the 24-hour measurement.

Histological Analysis of Pancreatic Islets, White Adipose Tissue, and Liver

Pancreata were collected, fixed overnight in 10% neutral formalin solution, embedded in paraffin, sectioned at 4 μm and stained with hematoxylin and eosin (H&E). Pancreatic sections were immunostained for β cells using guinea pig anti-swine insulin polyclonal antibody (Dako). Pancreatic sections were deparaffinized in xylene, quenched in 3% H₂O₂/H₂O for 5 min. Antigens were retrieved by boiling in citrate buffer (pH:6.0) for 12 min. Sections were then blocked in 5% goat serum/PBS for 30 min and incubated with the primary rabbit polyclonal antibody (1/500, Vector Laboratories) overnight at 4° C. Sections were developed using 3,3′-Diaminobenzidine (DAB) and counterstained with hematoxylin. To evaluate cell sizes or numbers, 4 to 10 sections (each 15 μm apart) were analyzed using a 40× objective on a Leica microscope outfitted with a CCD camera (SONY) and the Osteomeasure software. β-cell area represents the surface positive for insulin immunostaining divided by the total pancreatic surface. Ten mice were analyzed per condition.

Flow Cytometry Cell Sorting

Peripheral blood mononuclear cells (PBMCs) were isolated by density gradient centrifugation using Ficoll-Hypaque and were counted with Tryptan blue for viability using a hemacytometer. For flow sorting, PBMCs were resuspended in flow staining buffer at 1×10⁶/ml and labeled with polyclonal anti-human osteocalcin. After 30 minutes incubation at 4° C., the cells were washed twice and the following fluorochrome-conjugated primary and secondary antibodies were added: FITC conjugated AffinityPure IgG f(ab′)₂ fragment donkey anti-goat antibody, APC conjugated anti-CD45 and phycoerythrin (PE) conjugated anti-CD146. After 30 minutes incubation, cells were washed twice using flow buffer. Flow sorting was performed using FACSAria (BD). Sorted CD45^{low}/OCN⁺/CD146⁺ cell populations were then stored in RLT buffer at −80° C. for extraction of RNA.

Statistical Analyses

Results are given as means±SEM. Statistical analyses were performed using unpaired two-tailed Student's t or a one way ANOVA (Student-Newman-Keuls) for more than 2 groups.

### Example 1

### Lcn-2 is a Novel Osteoblast-Derived Hormone Regulating Energy Metabolism

We have previously reported that a transcription factor, FoxO1, acts on osteoblasts to regulate whole body glucose metabolism (Rached et al. J. Clinical Investigation, 2010). To identify new osteoblast-derived hormones, comparative microarray analysis in osteoblasts from FoxO1 knockout and wild type mice was conducted. The results taken together show that Lcn-2 is a novel osteoblast-derived hormone regulating energy metabolism. It was discovered that Lcn-2 was 2-fold upregulated in FoxO1-deficient osteoblasts as compared to wild type osteoblasts. Serum osteocalcin levels were also 2-fold upregulated in mice with osteoblast-specific deletion of FoxO1 (FoxO1_{osb}−/−). In addition, it was more highly upregulated in osteoblasts than in any other cell type tested, including adipocytes, where it was first identified. Indeed Lcn-2 was also able to dramatically increase insulin 1 and insulin 2 production and secretion in Ins 1 pancreatic beta cells in vitro.

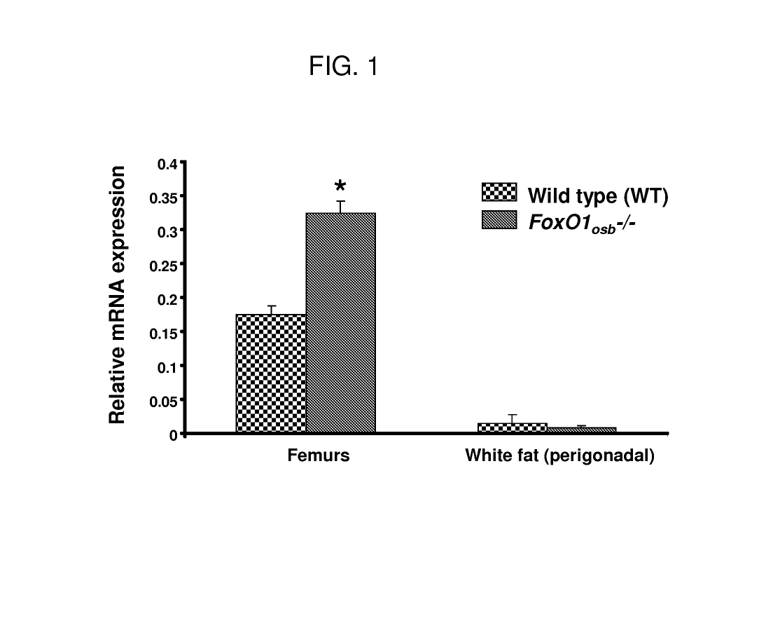

Lcn-2 mRNA expression is almost doubled in the femur of 1-month old female FoxO1_{osb},−/− mice compared to wild type mice. FIG. 1. By contrast only miniscule amounts of Lcn-2 mRNA were detected in white adipose tissue (WAT) of either wild-type or FoxO1_{osb}−/− mice. Consistent with this, is the observation that Len-2 serum levels are increased in FoxO1_{osb},−/− mice of different genetic background: the level of Lcn-2 is about 1.8 times higher in C57BL FoxO1_{osb}−/− mice than in C57BL wild type mice; and it is about double in Agouti mice of mixed background compared to wild type. FIG. 2. Lcn-2 has been identified as a secreted protein expressed in osteoblasts. Lcn-2 levels were measured in the supernatant of calvaria-derived osteoblasts that were placed in culture for 8 hours. This is a very short time point, but even so about 0.110 ng/m of Lcn-2 was measured, which comparable to osteocalcin levels under the same conditions. It is also known that Lcn-2 is a secreted protein and it is elevated in the serum of the FoxO1_{osb}−/− mice.

### Example 2

### Lcn-2 is Preferentially Expressed in Bone

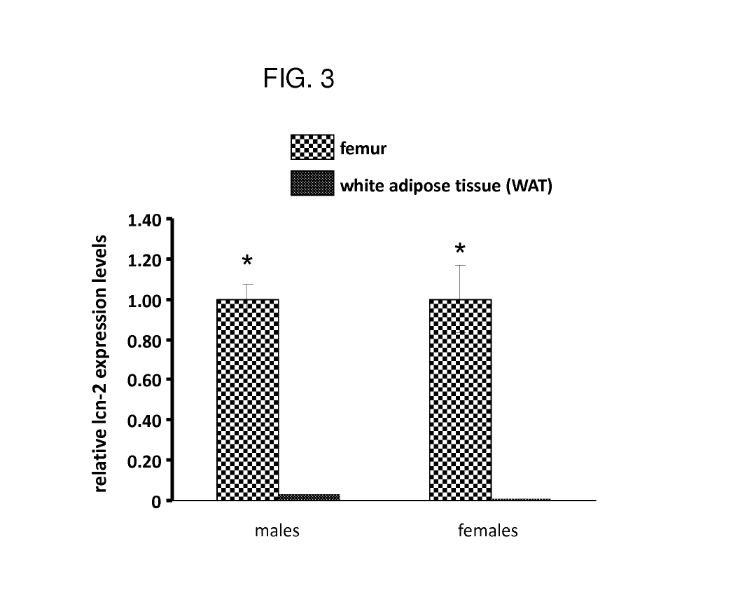

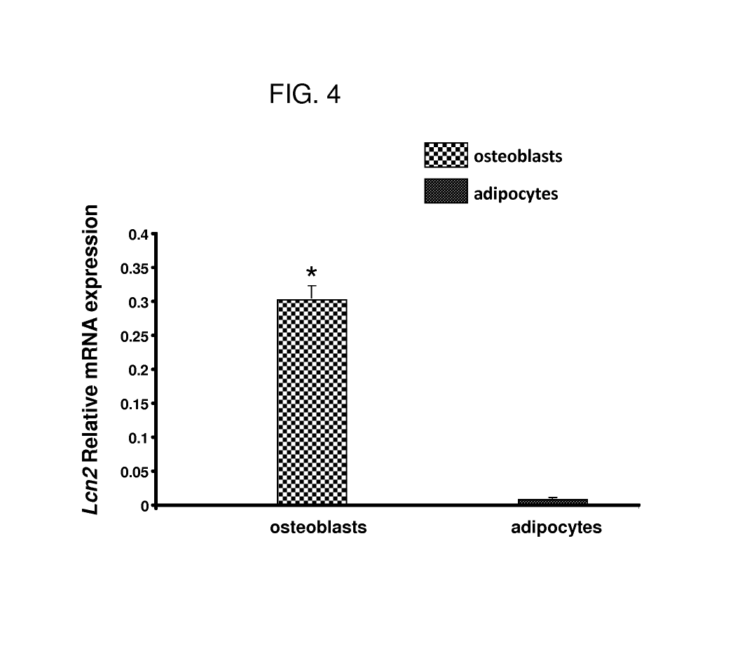

The relative level of Lcn-2 mRNA expression in bone is about 33 times higher compared to WAT in FoxO1−/− mice (FIG. 3); and the relative levels of Lcn-2 mRNA in osteoblasts is about 64-fold higher than it is in WAT (FIG. 4). Whole bone, in addition to osteoblasts, contains osteoclasts and osteocytes. Although the bone marrow was flushed before the tissue was analyzed in gene expression measurements, some bone marrow cells remain, so whole bone represents a heterogeneous cell population. Lipocalin-2 is also preferentially expressed in bone marrow-derived stromal cells from FoxO1−/− mice where the levels are 5-fold higher than in adipocytes of (FIG. 5).

### Example 3

### Lcn-2 Treatment in Ins 1 Pancreatic Cells Affects Expression of Certain Genes

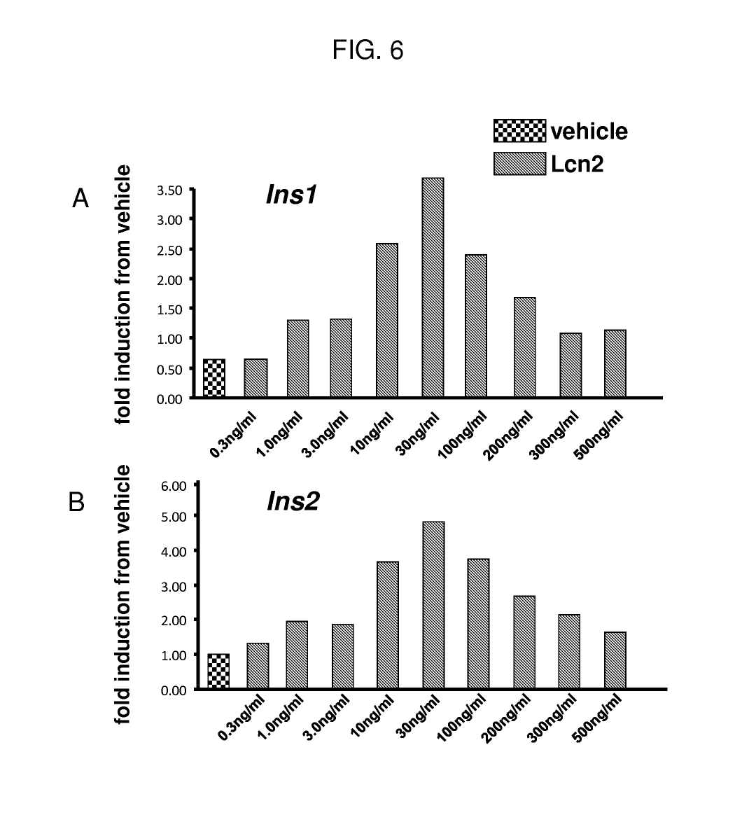

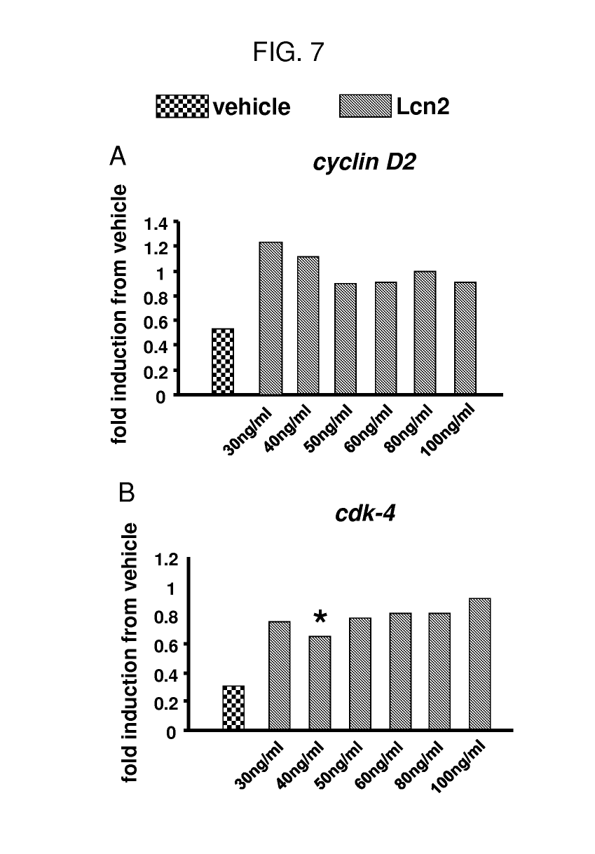

To study the role of Lcn-2 on insulin production, in vitro assays were conducted in which Ins1 pancreatic cells were treated with varying amounts of recombinant Lcn-2 (rLcn-2). rLcn-2 caused a dose-dependent upregulation of both proinsulin Ins1 and Ins2. Increased proinsulin expression was elicited beginning with an amount of 1 ng/ml to a maximum 3.5-fold increase for Ins1 and a 5-fold increase of Ins2 with application of 30 ng/ml Lcn-2. Higher concentrations of 100 to 500 ng/ml progressively reduced Ins1 and Ins2 expression to near baseline levels (FIG. 6). Lcn-2 in an amount of 30 ng/ml also increased expression of cyclin d2 (3-fold) and cdk 4 (3.2-fold), markers of cell proliferation, in Ins1 cells. Further increases in Lcn-2 did not further increase expression of the markers (FIG. 7). FIG. 7 shows increase in the expression of proliferative genes which indicates an increase in cell proliferation in vitro.

### Example 4

### Levels of PPARa and PGC-1a Affected By Lcn-2 Administration

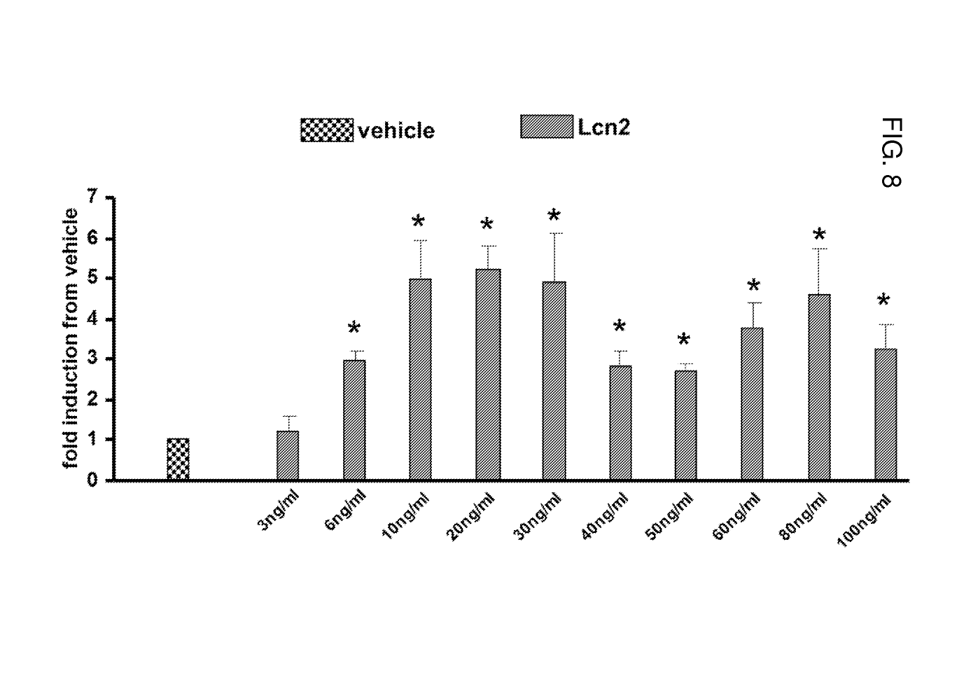

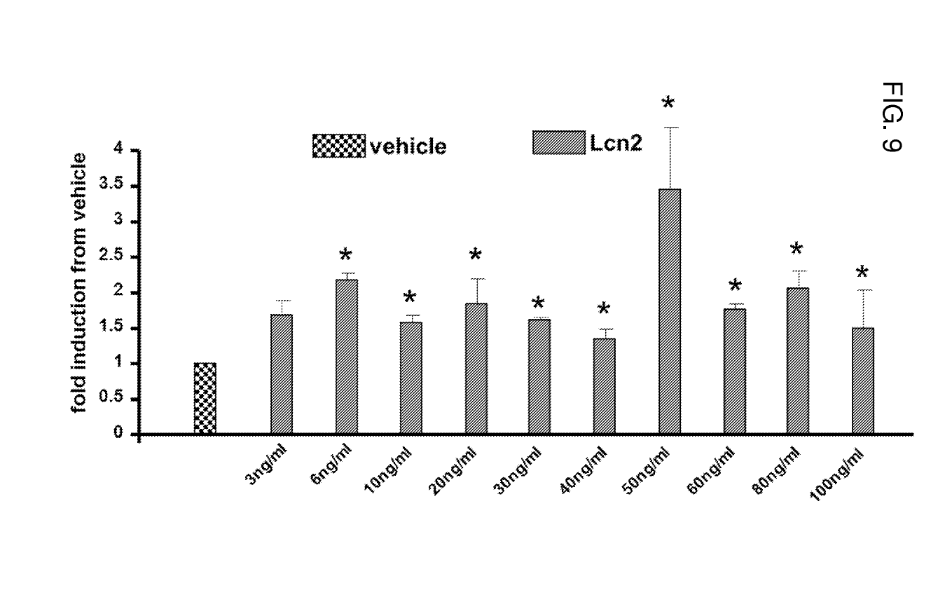

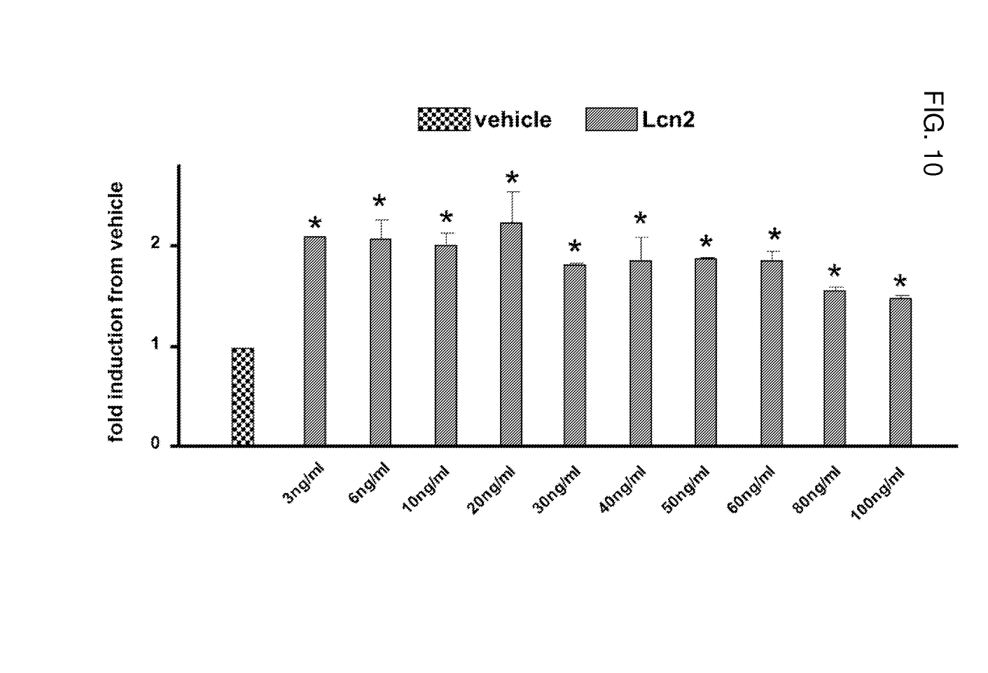

The level of peroxisome proliferator-activated receptor-alpha (PPARα), a key regulator of fatty acid oxidation in skeletal muscle, was increased nearly 3-fold by 6 ng/ml Lcn-2 to a maximum of more than 4-fold by 10-30 ng/ml in C2C12 myocytes in vitro (FIG. 8). The level of PGC-1a, a coactivator of the insulin target PPARa, was also increased in C2C12 myocytes by about 1.7-fold by 3 ng/ml Lcn-2 to a maximum of about 3.5-fold with 50 ng/ml; higher amounts did not further increase, but rather reduced expression. PPARa is highly expressed in metabolically active tissues including brown fat, skeletal muscle and heart; it is also involved in mitochondrial biogenesis and increased mitochondrial respiration (FIG. 9). Further, Nrf-1, a Pgc-1α target, was doubled by 3 ng/ml Lcn-2 in C2C12 myocytes; higher concentrations produced no further increase, but did not reduce expression of Nrf-1 (FIG. 10).

### Example 5

### Lcn-2 Administration Increases Mcad Expression

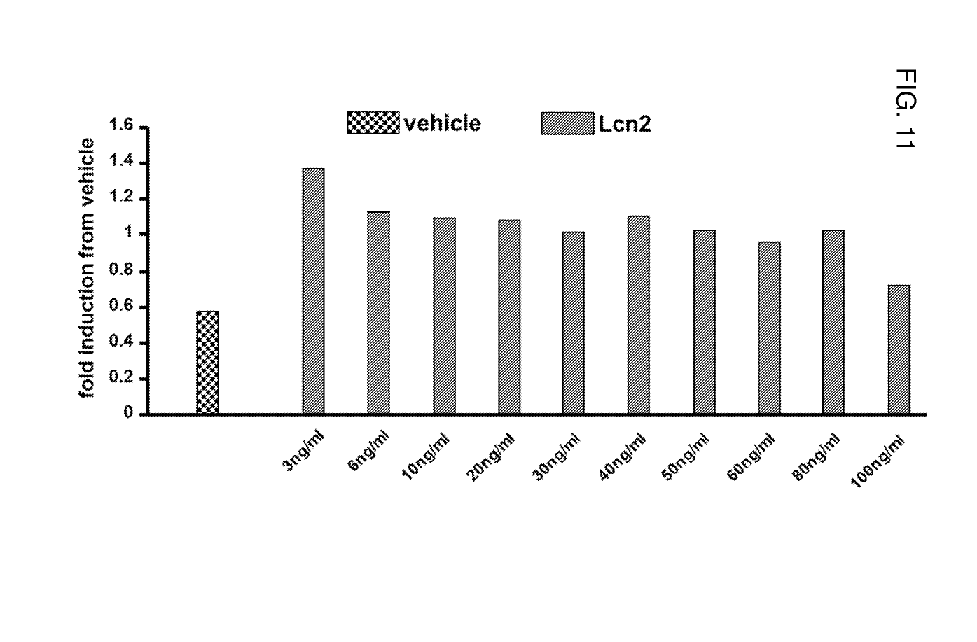

Medium-chain acyl-CoA dehydrogenase (Mcad) was increased more than 3-fold by 3 ng/ml Lcn-2; in C2C12 myocytes; higher concentrations produced no further increase, but did not reduce expression of Mcad. (FIG. 11). MCAD deficiency is a common inborn error of mitochondrial fatty acid oxidation. All three genes (PPARα, PGC-1α and Mcad) indicate an increase in mitochondrial activity which means increased energy expenditure, which means the animal burns more fat. They are also all involved in increased in fatty acid oxidation which leads to a decrease in lipogenesis, increase in insulin sensitivity and improved glucose transport. Specifically MCAD is involved in the first step of the mitochondrial β-oxidation of fatty acids.

Fatty acid oxidation is essential for energy production. This metabolic pathway is complex and comprises as many as 20 individual steps including uptake and activation of fatty acids by cells, the carnitine cycle and the beta-oxidation spiral, with various enzymes required for the oxidation of unsaturated fatty acids. Fatty acid oxidation disorders are a group of inherited metabolic conditions that lead to an accumulation of fatty acids, and a decrease in cell energy metabolism. Each fatty acid oxidation disorder is associated with a specific enzyme defect in the fatty acid metabolic pathway and affects utilization of dietary and stored fat. All of these disorders are inherited in an autosomal recessive pattern. Inherited enzymatic defects in the pathway lead to accumulation of fatty acids or a decrease in cell energy metabolism and result in the clinical manifestations of the disorder.

MCAD is the most common of the fatty acid oxidation disorders with an incidence of approximately one in 10,000 to 20,000 births. LCHAD and VLCAD are rare disorders with an estimated incidence of one in 100,000 births. There is a mild form of SCAD deficiency that appears to be quite common, but the clinical significance of this condition is unclear. Newborn screening includes testing for a panel of acylcarnitines. In some cases, an elevated level of a particular acylcarnitine may indicate the possibility of one of several different fatty acid oxidation disorders; the specific disorder cannot be determined without diagnostic further testing. It has been demonstrated that the following fatty acid oxidation disorders may be detected in newborn dried blood spot samples using this testing panel.
Carnitine/acylcarnitine translocase deficiency (CACT);
Carnitine palmitoyl transferase deficiency type II (CPT II);
Carnitine palmitoyl transferase deficiency type 1A (CPT1A);
Carnitine Uptake Defect (CUD); Glutaric aciduria type II (GA II)/Multiple acyl-CoA dehydrogenase deficiency (MADD);
Isobutyryl-CoA dehydrogenase deficiency (IBCD);
Medium chain acyl-CoA dehydrogenase deficiency (MCAD);
Long chain 3-hydroxyacyl-CoA dehydrogenase deficiency (LCHAD);
Short chain acyl-CoA dehydrogenase deficiency (SCAD);
Medium/Short chain L-3-hydroxyacyl-CoA dehydrogenase deficiency (M/SCHAD);
Trifunctional protein deficiency (TFPD); and
Very long chain acyl-CoA dehydrogenase deficiency (VLCAD).

Affected infants can be diagnosed in the neonatal period. Children with MCAD have a significant risk of death during the first or subsequent clinical episode of hypoglycemia. In most cases, the first episode arises following illness or fasting, and occurs in infancy or early childhood. Fatty acid oxidation disorders can cause recurrent episodes of hypoglycemia. Clinical findings may include lethargy, hypotonia, failure to thrive, persistent vomiting, hepatomegaly, rhabdomyolysis and Reye syndrome-like episodes.

Early diagnosis and treatment is essential for an improved prognosis. If left untreated, these conditions may result in significant disability and, ultimately, death. Most of these conditions are chronic, with life-long episodes of hypoglycemia. In some of the more severe infantile forms, there is a very poor prognosis. For most fatty acid oxidation disorders, including MCAD, management involves avoidance of fasting and aggressive medical management during illness, especially if the child is vomiting or is not receiving adequate nutritional intake. At the time of intercurrent illness, the infant/child should be admitted for medical care, including administration of intravenous dextrose to prevent hypoglycemia. Supplemental carnitine, a low-fat diet and home glucose monitoring, may be prescribed depending on the specific disorder.

### Example 6

### Lcn-2 is Specifically Expressed by Bone Cells

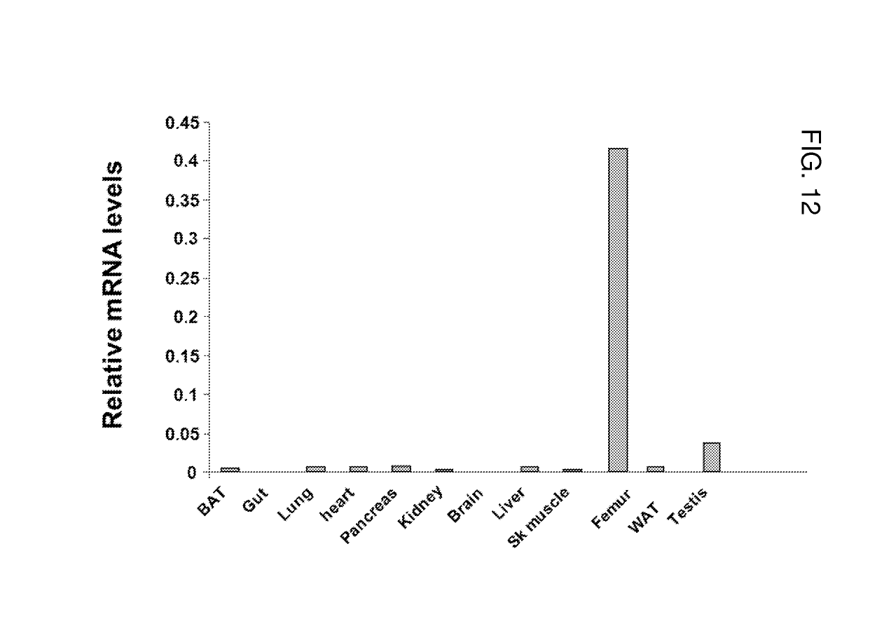

Gene expression analysis by quantitative real time PCR in a variety of mouse tissues indicated that Lcn-2 is specifically expressed by bone cells, with a 10-fold lower expression by the testis (FIG. 12).

### Example 7

### Lcn-2 Administration Increases Mrf-4 and Adiponectin Expression and Decreased Resistin Expression

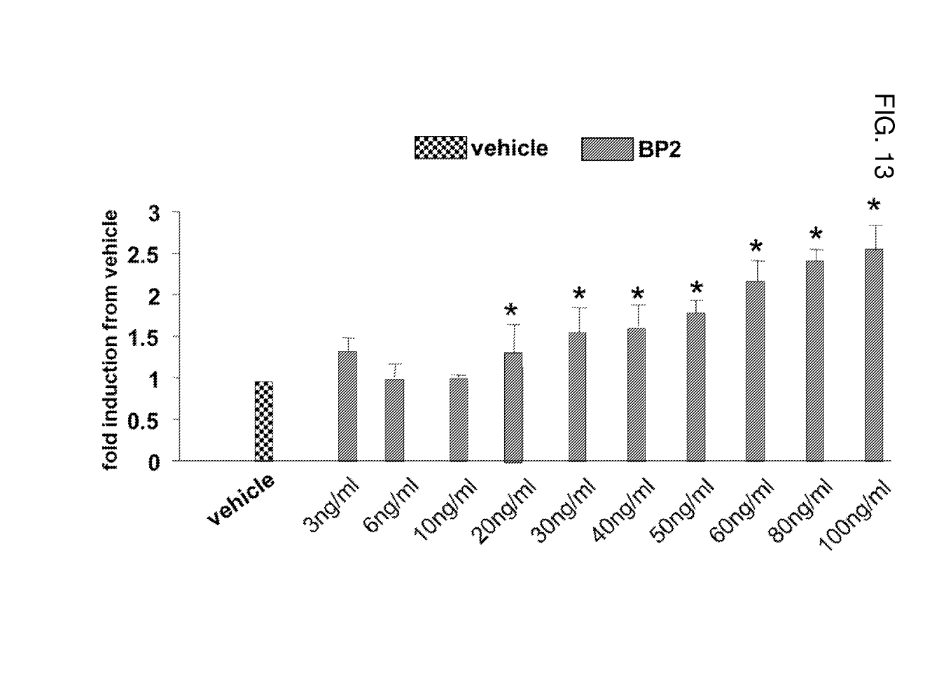

Additional in vitro treatment experiments with recombinant Lcn-2, performed as described earlier in this application, showed that Lcn-2 dose dependently increased the expression of Mrf-4, a gene that regulates myogenesis, in C2C12 myocytes (FIG. 13).

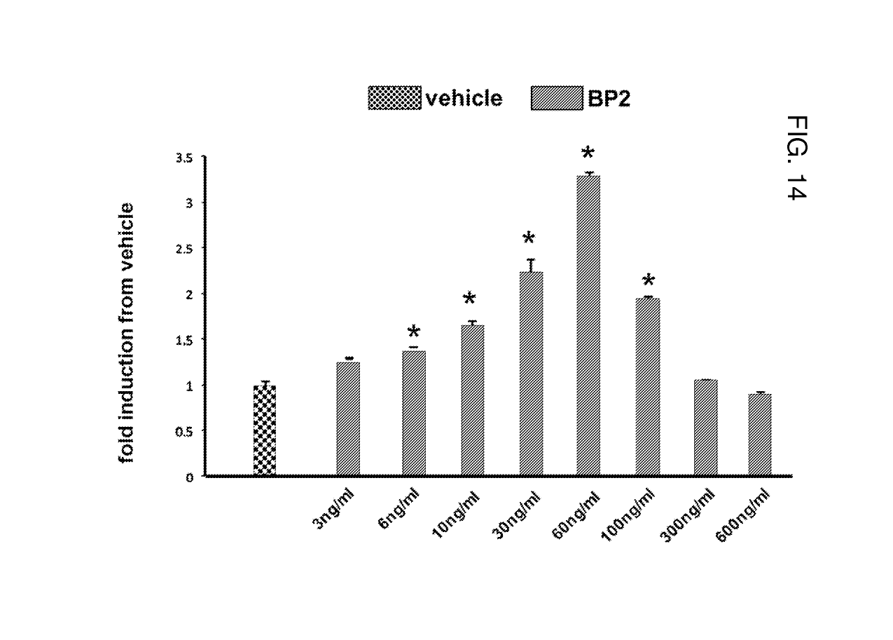

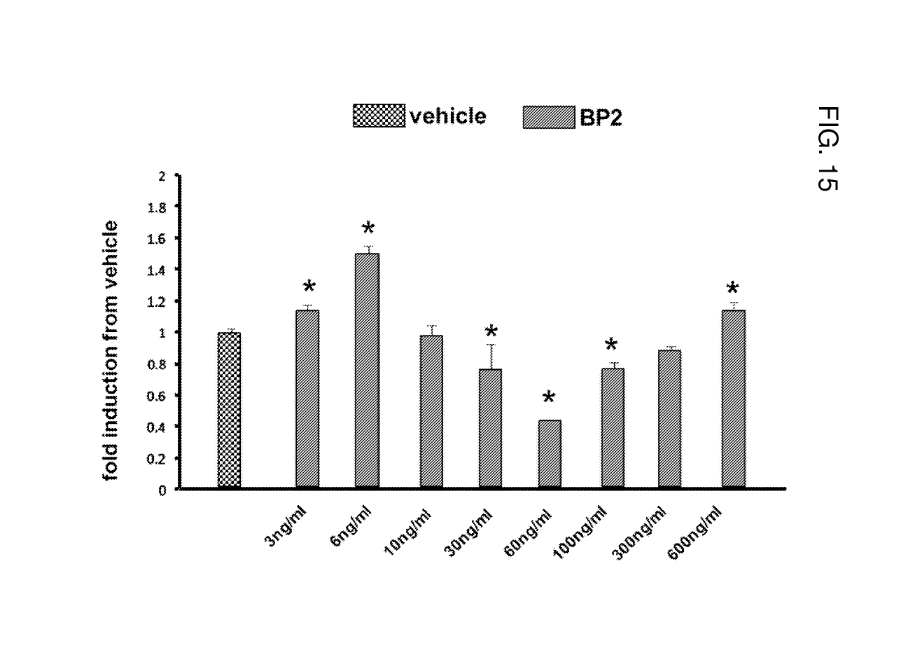

Lcn-2 also increased the expression of the insulin-sensitizing hormone Adiponectin in 3T3-L1 adipocytes (FIG. 14), whereas it decreased the expression of Resistin, an adipocyte-produced hormone that is associated with insulin resistance in 3T3-L1 adipocytes (FIG. 15).

### Example 8

### Improvement of Glucose Tolerance

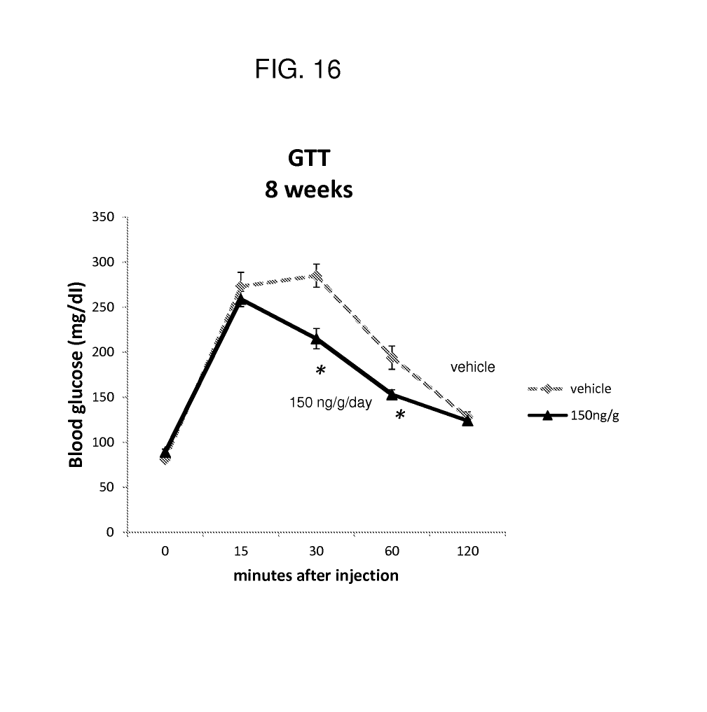

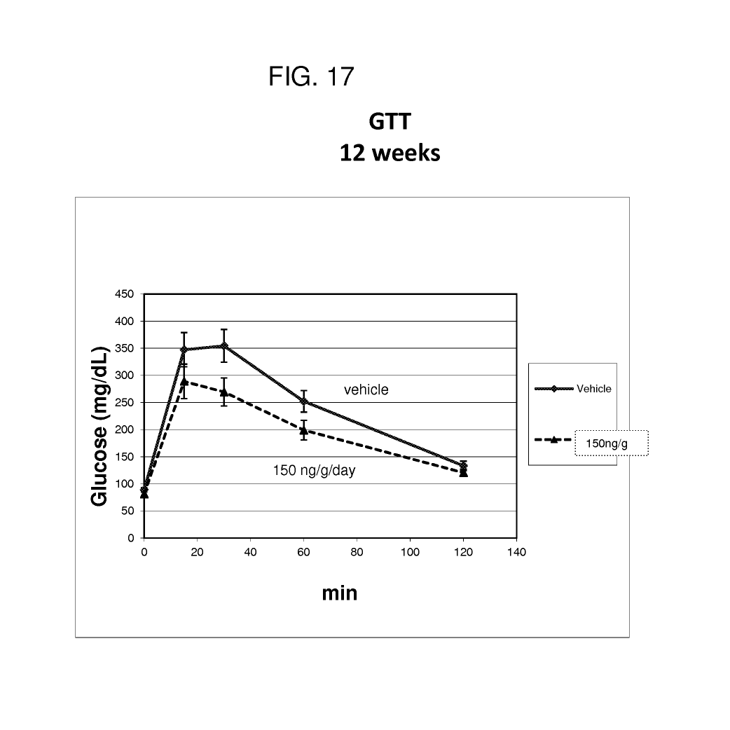

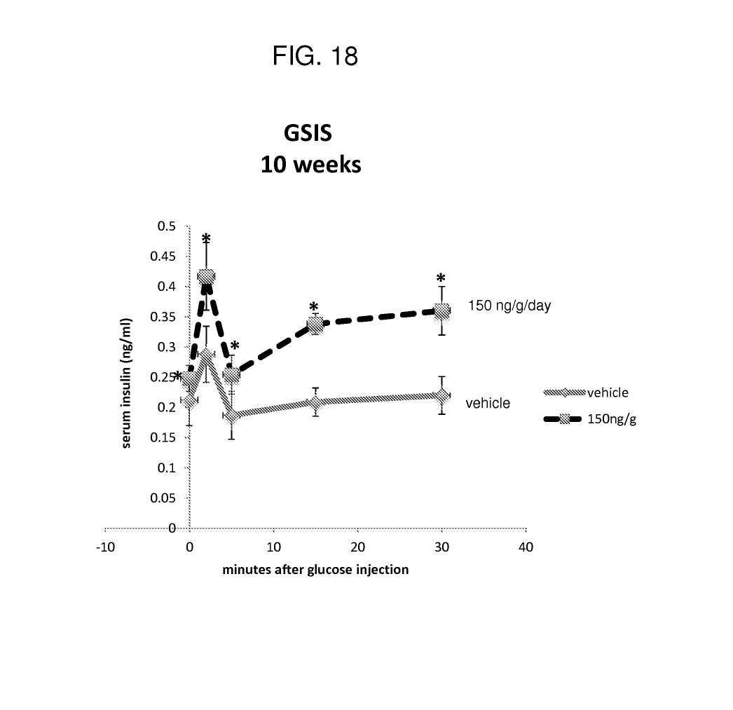

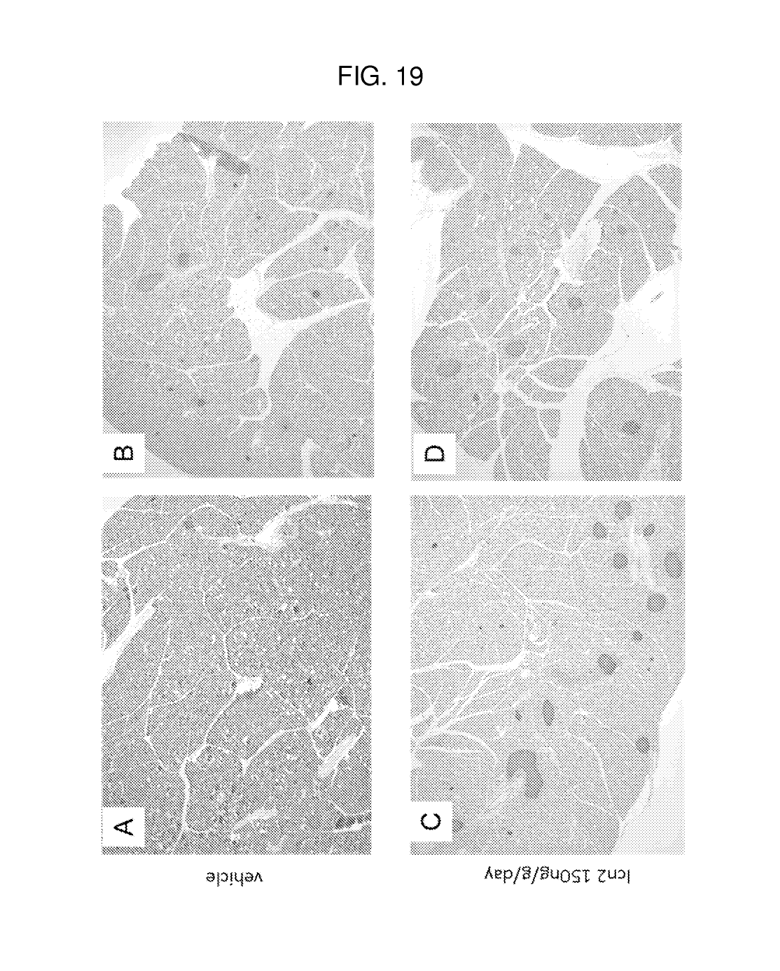

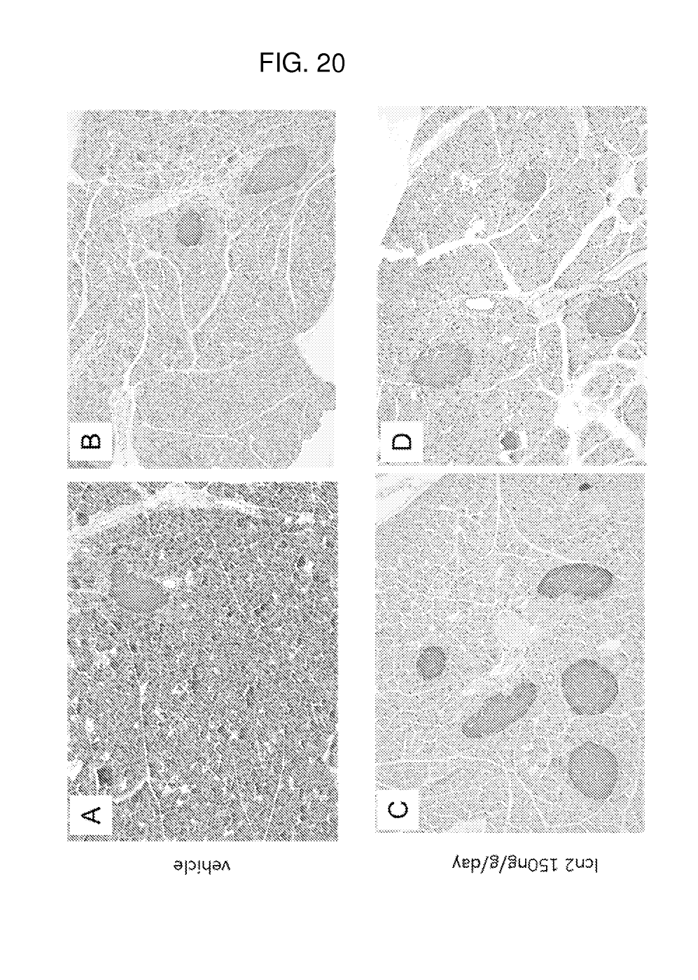

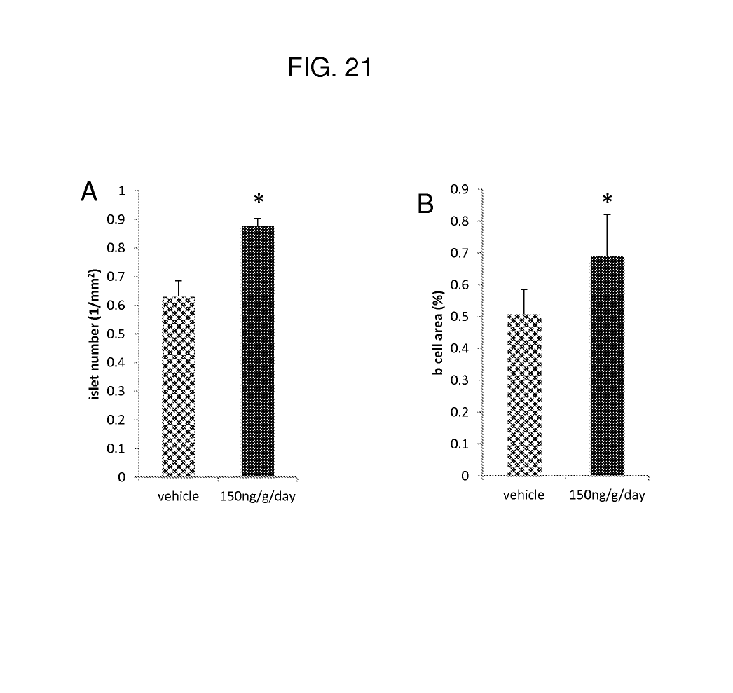

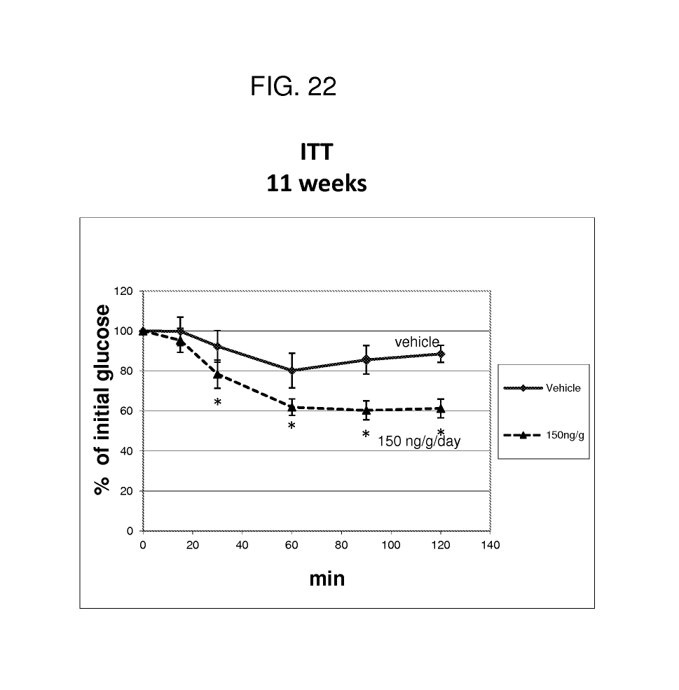

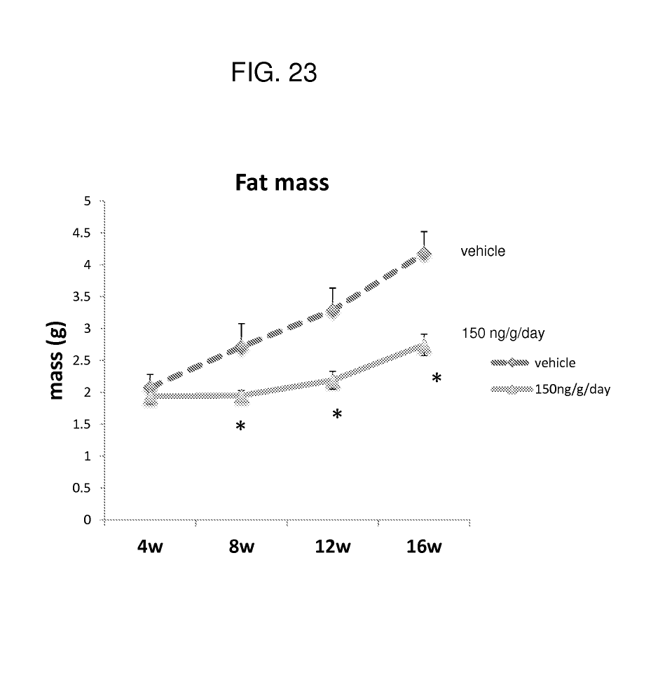

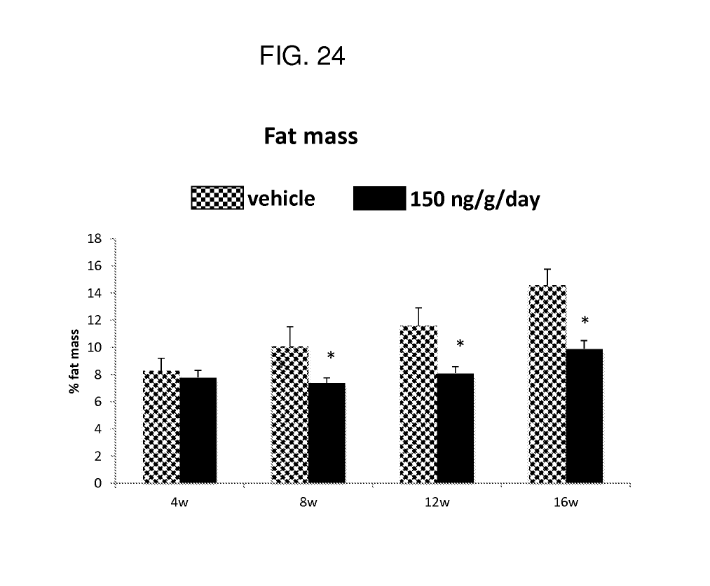

To test the in vivo potency of Lcn-2 in glucose metabolism, recombinant Lcn-1 was administered to healthy, wild-type mice at 8 weeks of age. Lcn-2 was administered by daily intraperitoneal injection. Three doses of Lcn-2: 50, 150 and 500 ng/g body weight were administered. In all end points measured we observed curves similar to those obtained by the in vitro treatments: The response reached a peak at 150 ng/g and then decreased at 500 ng/g. Without being bound by theory, this type of responses may be indicative of the type of Lcn-2 receptor. G-protein coupled receptors are known to mediate such types of responses by hormone ligands. At 8 weeks following treatment a glucose tolerance (GTT) test indicated that mice treated with Lcn-2 had improved glucose tolerance (FIG. 16). The improvement on glucose tolerance was also evident at 12 weeks following initiation of treatment (FIG. 17). Lcn-2 treated mice demonstrated higher insulin levels after glucose challenge at every time point measured. The GSIS test was performed at week 10 of treatment (FIG. 18). In agreement with the increased levels of serum levels in Lcn-2-treated mice, these same animals showed increased β-cell area and β-cell numbers (FIGS. 19-21). In addition, Lcn-2-treated mice showed improved insulin sensitivity as examined by an ITT test (FIG. 22). Starting at 4 weeks and throughout treatment, Lcn-2-treated mice demonstrated lower fat mass as compared to untreated animals (FIGS. 23-24). Whereas aging mice progressively gain fat mass, mice treated with Lcn-2 gained a lot less with a progressive increase in the difference between treated and untreated animals. Fat measurements were performed using an MRI machine. In addition to the beneficial effects in reducing body fat, Lcn-2 treatment also increased lean mass (FIG: 25). The increase was progressive from 4 to 16 weeks of treatment.

### Example 9

### Lcn-2 Administration Increases Energy Expenditure and Activity and Decreases Appetite

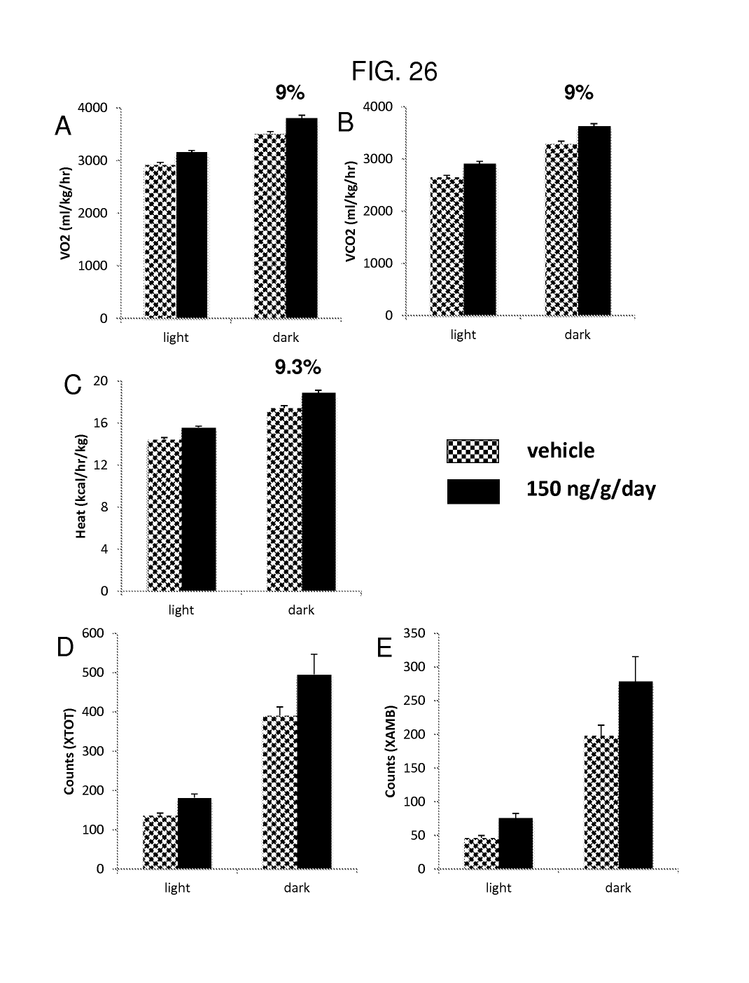

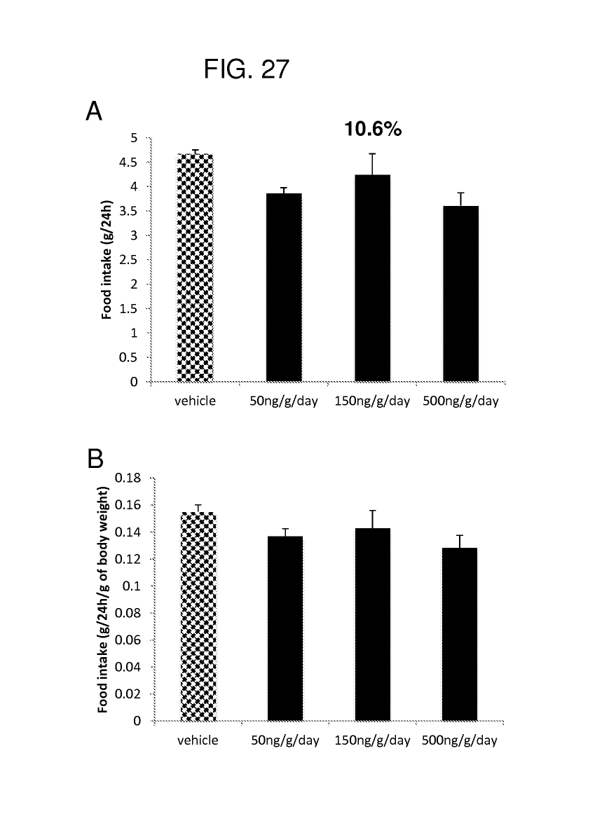

calorimetric measurements also indicated that Lcn-2 increased energy expenditure (measure by the volume of O₂ and CO₂, and by heat production) and also increased activity (FIG. 26). It also decreased appetite (FIG. 27).

### Example 10

### Expression of Perillipin, Triglyceride lipase (Tgl), and Lipoprotein lipase (Lpl) was Decreased by Lcn-2 Treatment

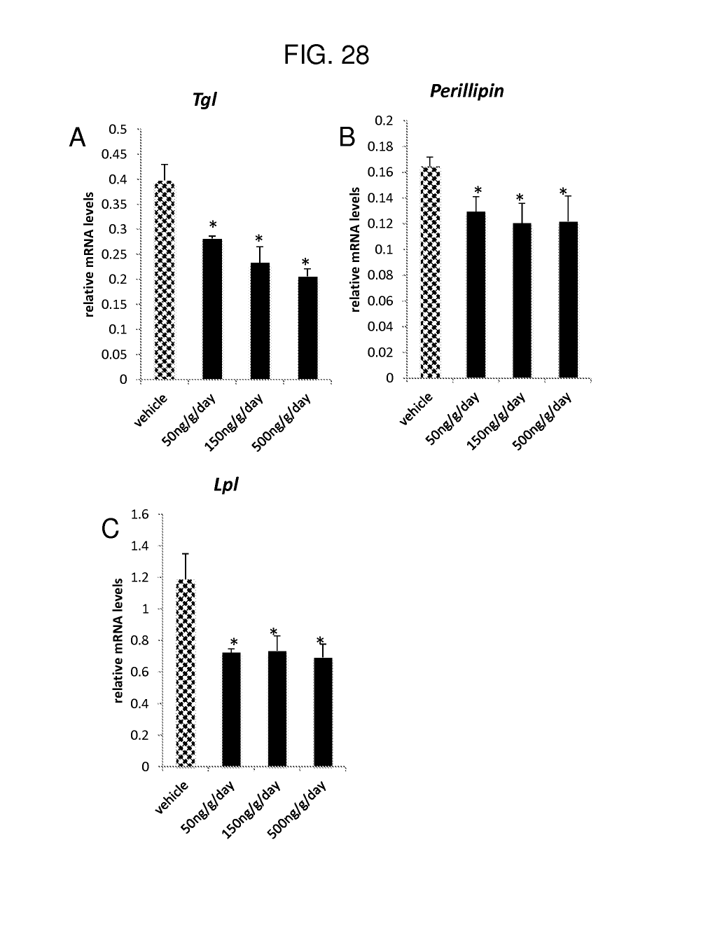

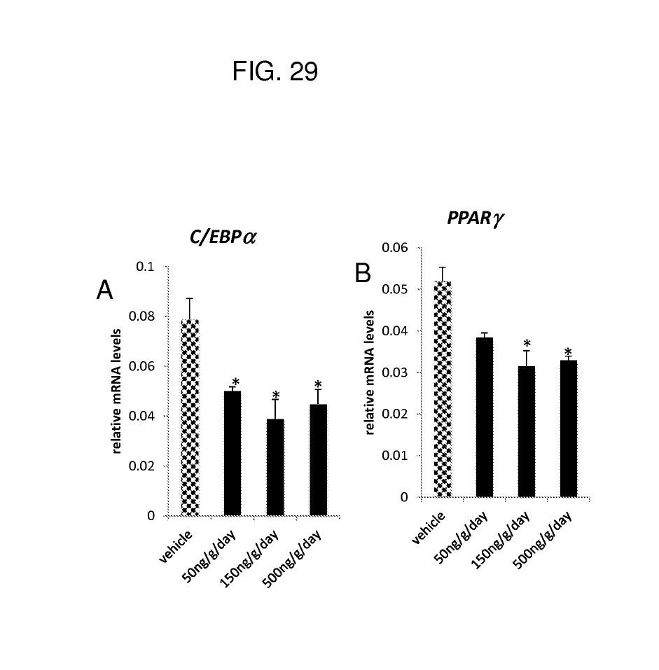

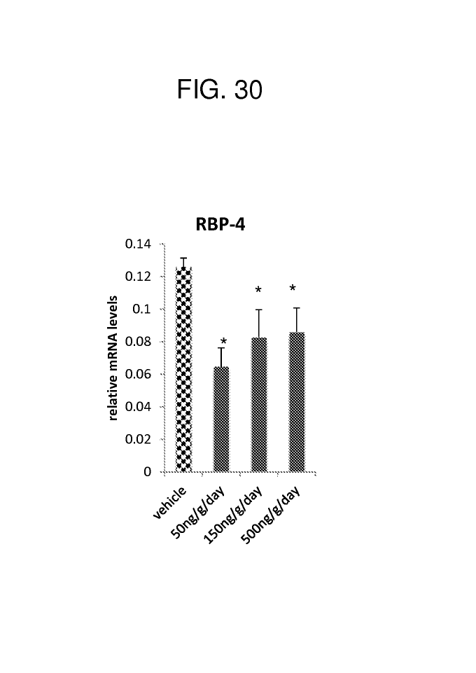

Gene expression analysis in several tissues following harvest of mice after 18 weeks of treatment was performed. We found that expression of two lipolytic genes Perillipin and Triglyceride lipase (Tgl), whose expression is inhibited by insulin, was decreased by Lcn-2 treatment (FIG. 28). Expression of Lipoprotein lipase (Lpl) involved in lipogenesis was also decreased (FIG. 28). These molecular changes indicated that lipolysis and lipogenesis and fatty acid uptake are decreased by Lcn-2 treatment. Lcn-2 also suppressed the expression of the two major genes promoting adipocyte differentiation, C/EBPα and PPARγ (FIG. 29). Lcn-2 treated mice also demonstrated a decrease in the expression of RBP-4 (Retinol binding protein 4) in white adipose tissue, an adipokine shown to promote insulin resistance and associated with obesity and diabetes in mice and humans (FIG. 30).

### Example 11

### Lcn-2 Administration Increases Gene Expression in Muscle

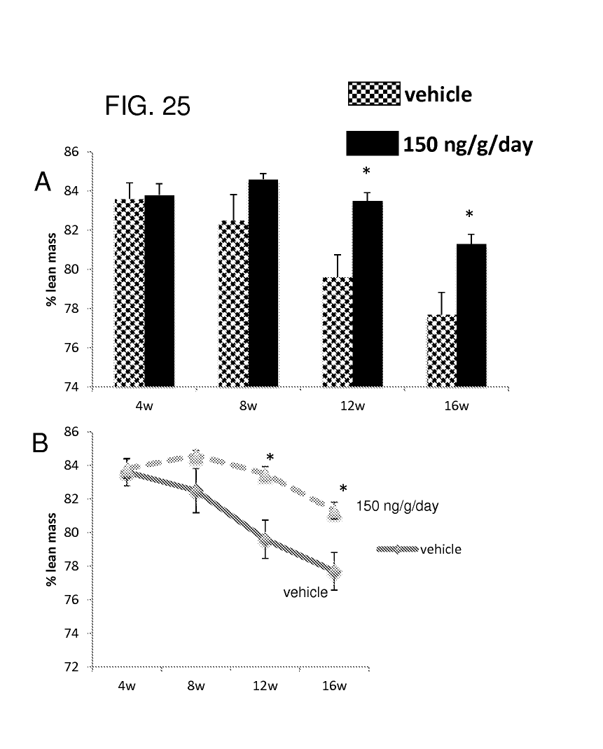

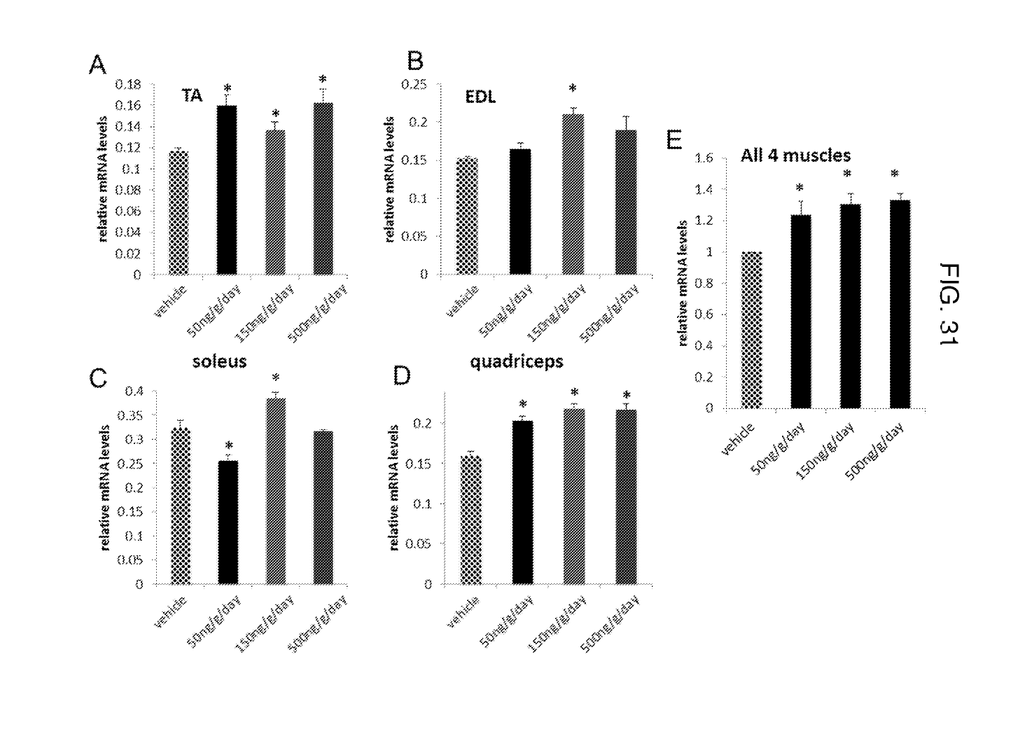

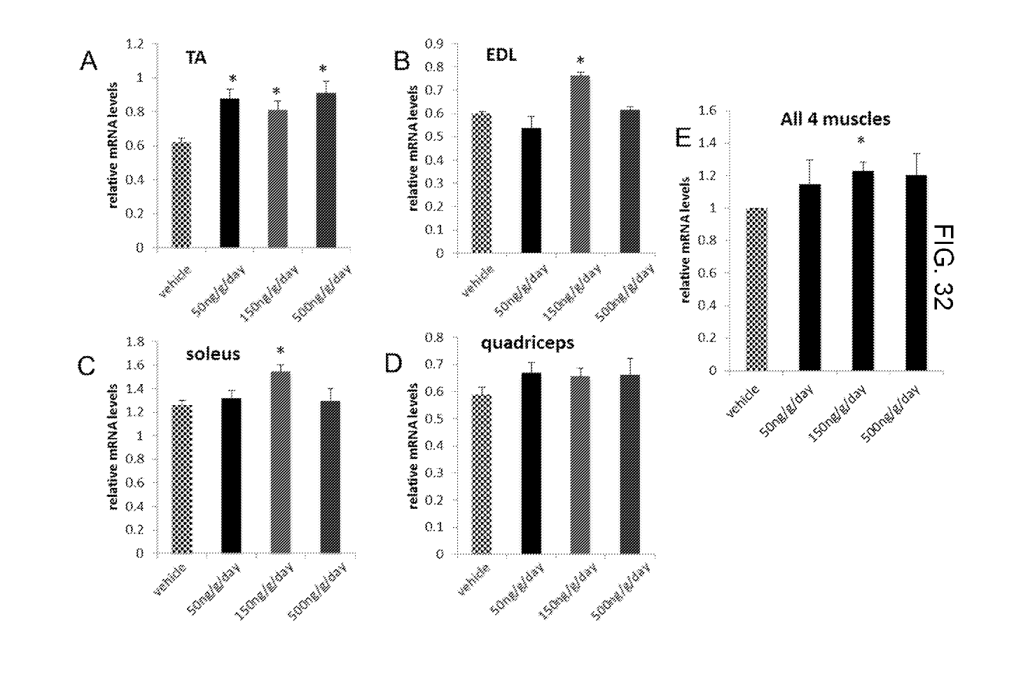

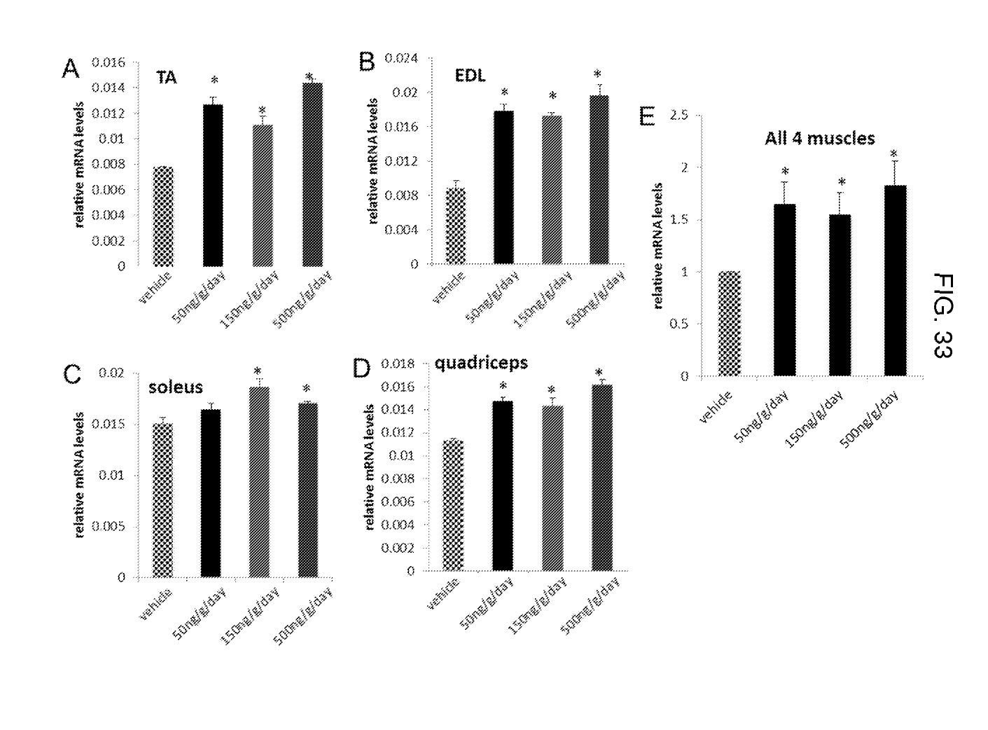

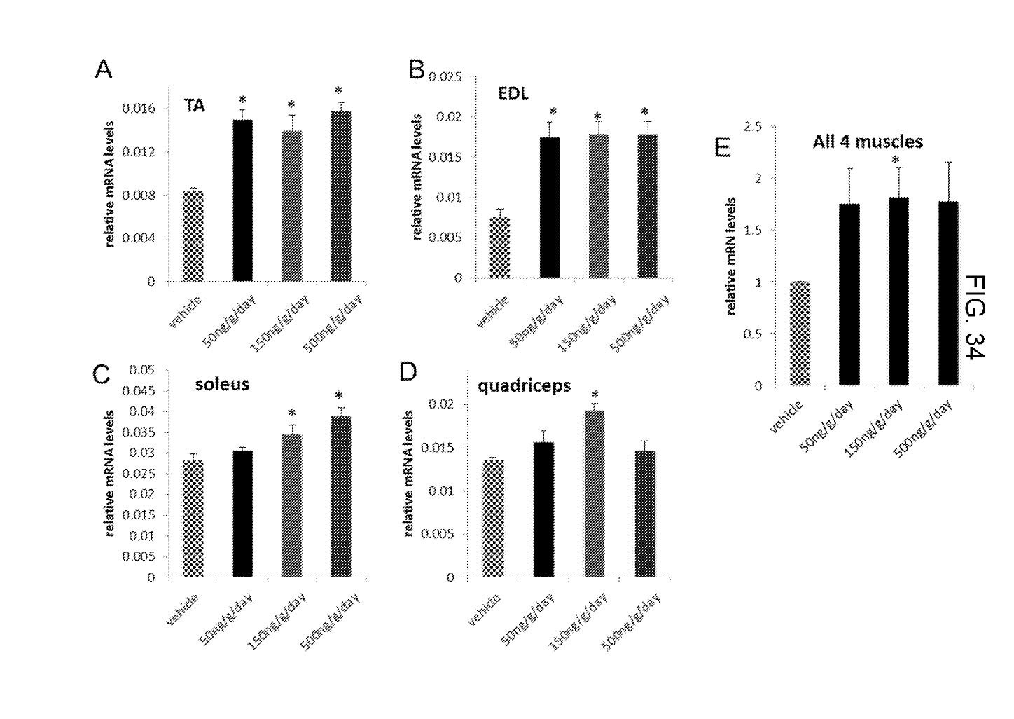

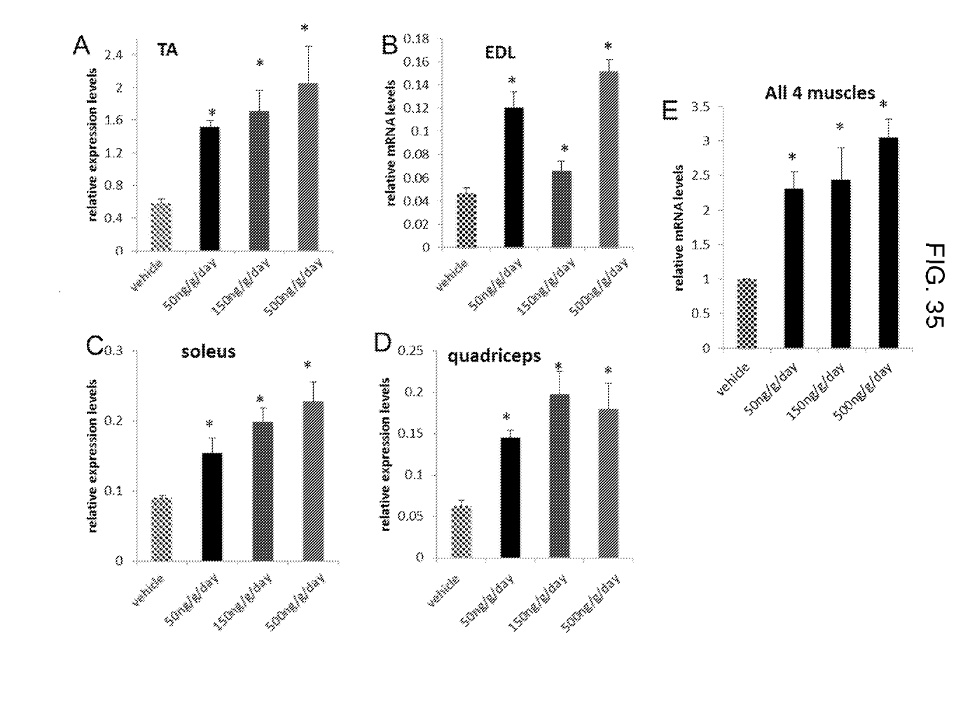

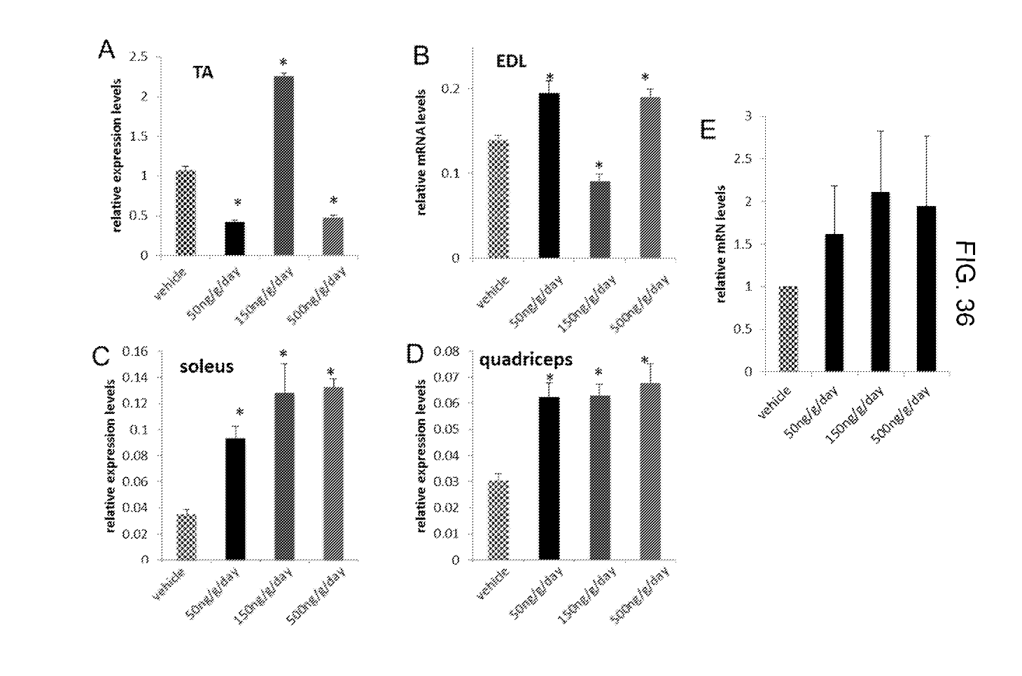

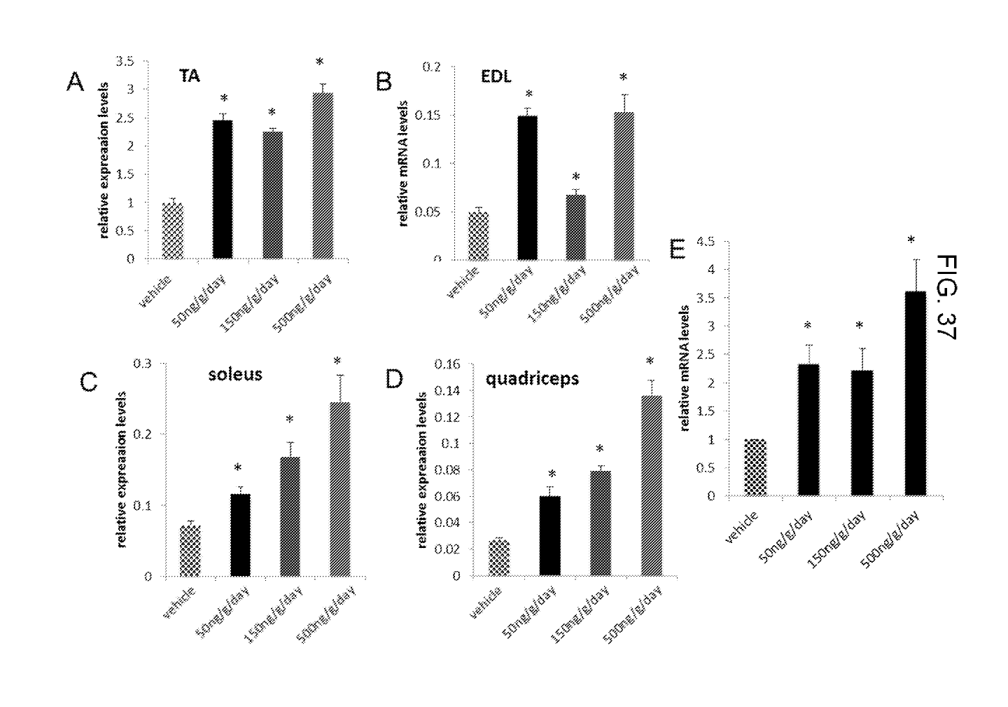

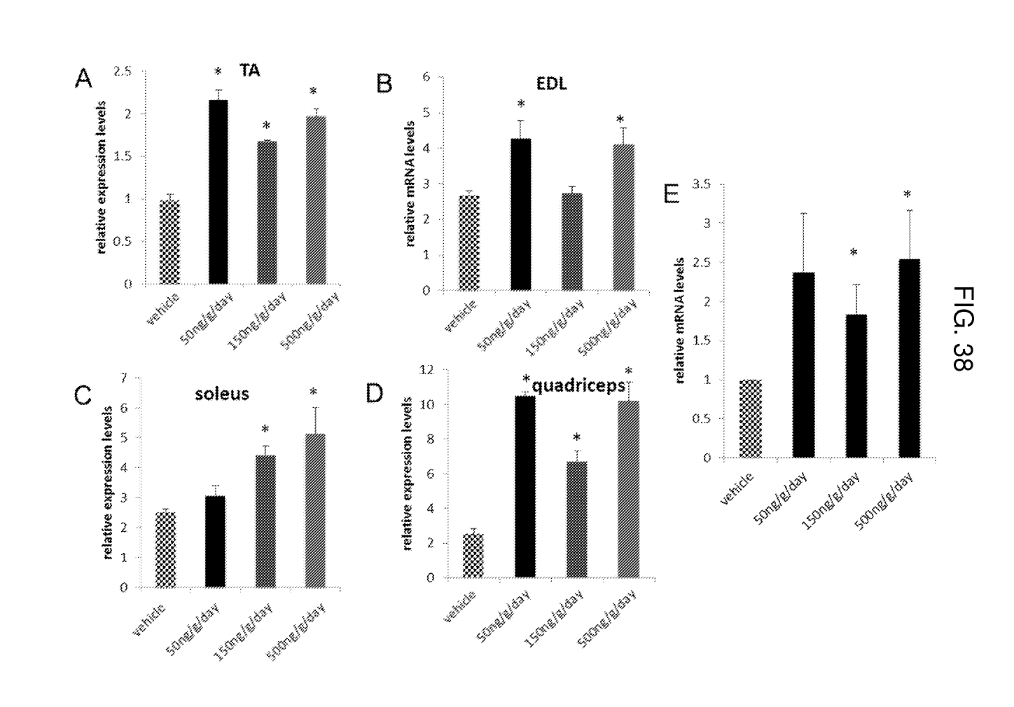

In muscle, Lcn-2 treatment increases the expression of genes that promote myogenesis and fatty acid beta-oxidation. Expression was measured in four 4 muscles: Tibialis anterior, Extensor digitorum longus, soleus and quadriceps. Results were also shown as cumulative data in all 4 muscles. Lcn-2 increased expression of acylcoA, the first and rate-limiting enzyme in the peroxisomal fatty acid beta-oxidation pathway, a PPARα target, stimulated by adiponectin (FIG. 31). It also increased expression of two Pgclα target genes, Nrfl and Mcad (FIGS. 32-33). Lcn-2 increased also the expression of UCP2, Mitochondrial uncoupling protein 2, (FIG. 34) which separates oxidative phosphorylation from ATP synthesis with energy dissipated as heat, and regulates mitochondrial membrane potential. Its increase indicates increase in mitochondrial activity and energy expenditure. All the key transcription factors involved in myogenesis, where upregulated by Lcn-2 treatment. Those include myoG, myoD, myf5 and mrf4 (FIGS. 35-38). Taken together, the in vivo observations in the increase in lean mass (FIG. 25) along with the increases in the expression of genes promoting myogenesis, fatty acid oxidation and mitochondrial activity in vivo, (FIGS. 31-38) as well as in vitro testing suggest that Lcn-2 is a potent regulator of myogenesis and muscle sensitivity.

### Example 12

### Lcn-2 Administration Fails to Induce an Inflammatory Response

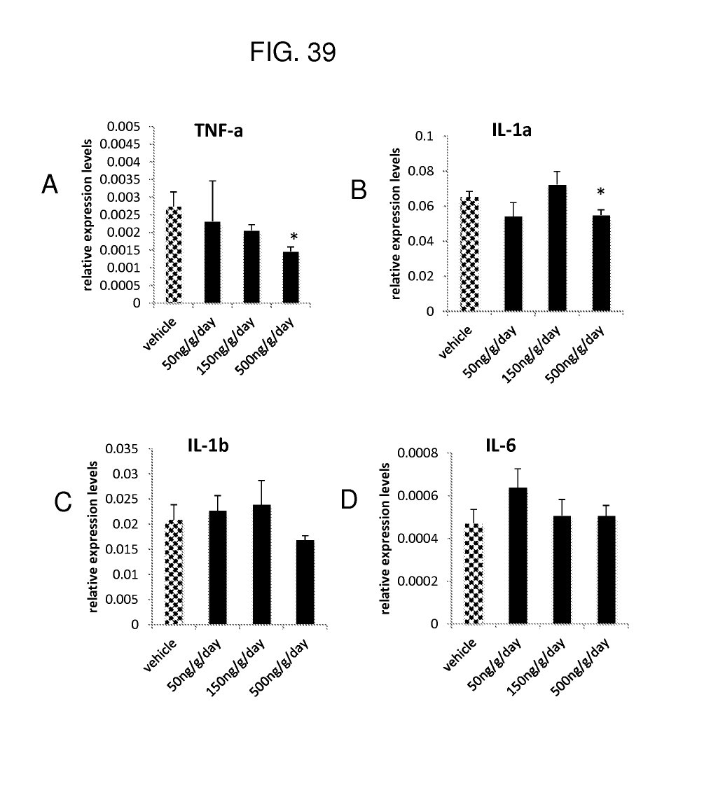

Lcn-2 treatment did not cause any inflammatory responses at either of the 3 concentrations tested as shown by the lack of any changes in the expression of the cytokines TNFα, IL-1a, IL-1b and IL-6 in the liver (FIG. 39).

### Example 13

### Lcn-2 Serum Levels Affected by Osteoblasts

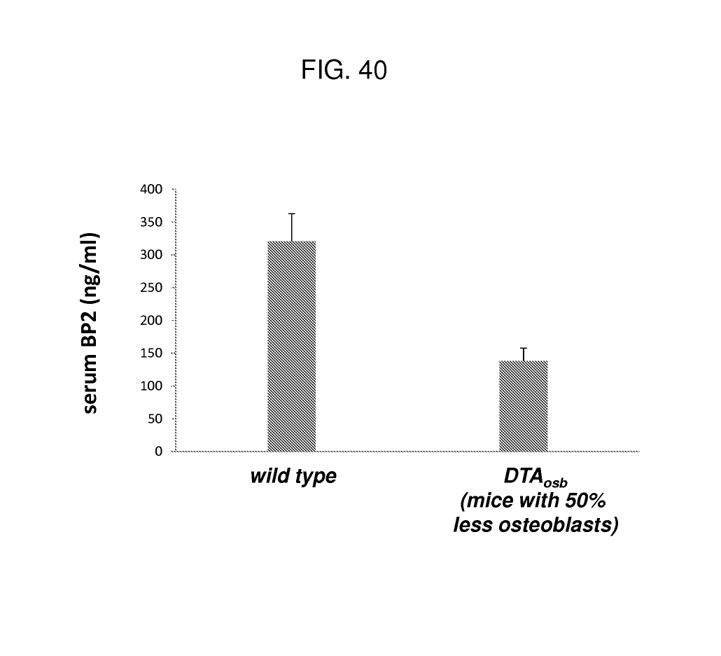

In addition serum levels of Lcn-2 were decreased by 50% in mice lacking 50% of osteoblasts (FIG. 40).

### Example 14

### Lcn-2 Expression Decreased in Diabetic Patients

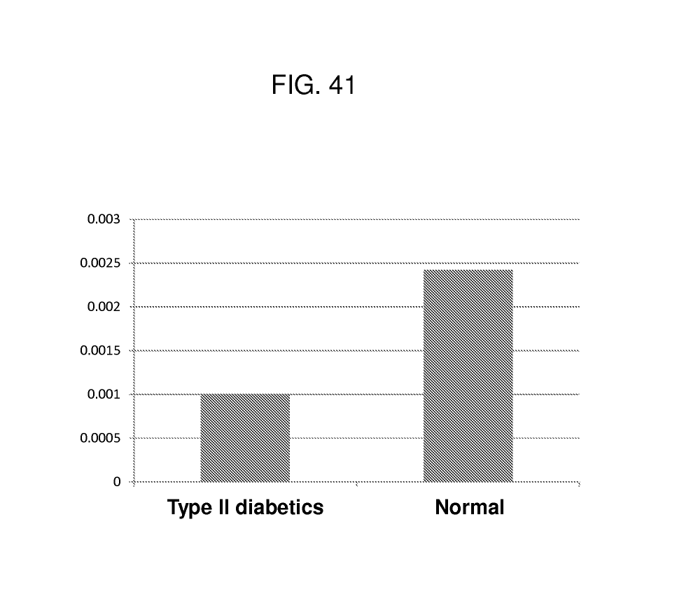

Finally, expression of Lcn-2 was decreased in osteoblastic cells obtained from diabetic (type II diabetes) patients as compared to healthy controls. (FIG. 41). Osteoblastic cells were identified as a CD45low, OCN+/CD146+ population derived from peripheral blood.

### Example 15

### Lcn-2 Injected Mice Exhibit Significantly Lower Glucose Levels and Higher Body Weights Compared to STZ-injected Mice

STZ is a glucosamine-nitrosourea compound that is used clinically as a chemotherapeutic agent in the treatment of pancreatic β-cell carcinoma as it is particularly toxic to pancreatic β-cells resulting in hypoinsulinemia and hyperglycemia. Its selectivity for β-cells is associated with preferential accumulation in β cells after entry through the GLUT2 glucose transporter, where it augments the generation of reactive oxygen species provoking ultimately β-cell death by apoptosis. Insulin producing β cells appear particularly vulnerable to oxidative stress due to their low levels of ROS-scavenging enzymes. To check whether enhanced lipocalin-2 serum levels could ameliorate hyperglycemia caused by pancreatic b-cell failure, 9-week old male mice) were injected with a single high dose of STZ (150 mg/kg of body weight) to induce b-cell death. Eight days later, half of the STZ-injected mice were injected daily with 150 ng/g recombinant Lcn-2. Blood glucose was measured every 48 h with a glucometer. STZ induced diabetes (fed blood glucose>250 mg/dl) in 50% of the mice injected by day 2, 56.25% by day 4 and 62.5% by day 6 which remained stable thereafter, with blood glucose levels ranging from 400-500 mg/dl. 20 days after the STZ injection and 12-18 days after lcn-2 treatment, lcn-2 injected mice had significantly lower glucose levels and higher body weights compared to STZ-injected mice. (Unpublished data).

### Example 16

### Lcn-2 Biologically Active Fragments

In order to identify Lcn2 biologically active fragments, three binding sites that are required for forming the ligand pocket and ligand binding were mutated. These are conserved among mouse, rat and human. The Lcn-2 protein was broken into two fragments: 1-120 amino acids (approximately 11 kDa because it is minus the 20 amino acids of signal sequence; and 121-200 amino acids (approximately 12 kDa). The conserved sites among Lcn2 and GPCR binding ligands were identified. Those have been determined for some GPCR ligands such as glucagon and are approximately 30-50 amino acids=2-3 kDa.

The entire contents of the following references are hereby incorporated by reference as if fully set forth herein, except for terminology that is inconsistent with the terminology used herein.
1. Centers for Disease Control and Prevention. National Diabetes Fact Sheet: national estimates and general information on diabetes and prediabetes in the United States, 2011. Atlanta, Ga.: U.S. Department of Health and Human Services, Centers for Disease Control and Prevention, 2011.
2. Data from the National Health and Nutrition Examination Survey (NHANES): Data Brief, No. 82, January 2012, Ogden, C., et al., “Prevalence of Obesity in the United States, 2009-2011.
3. Lee, N. K., Sowa, H., Hinoi, E., Ferron, M., Ahn, J. D., Confavreux, C., Dacquin, R., Mee, P. J., McKee, M.D., Jung, D. Y. Zhang Z, Kim J K, Mauvais-Jarvis F, Ducy P, Karsenty G 2007, Cell 130:456-469.
4. Ferron, M., Wei, J., Yoshizawa,T., Del, F. A., DePinho, R. A., Teti, A., Ducy, P., and Karsenty, G. 2010, Cell 142:296-308
5. Rached, M. T., Kode, A., Silva, B. C., Jung, D. Y., Gray, S., Ong, H., Paik, J. H., DePinho, R. A., Kim, J. K., Karsenty, G., Kousteni, S. 2010, J. Clin. Invest 120:357-368
6. Soriano P. Generalized lacZ expression with the ROSA26 Cre reported strain. Nat. Genet. 1999; 21: 70-71.
7. Beck, E, Ludwig, G, Auerswald, E A, Reiss, B, Schaller, H. Nucleotide sequence and exact localization of the neomycin phosphotransferase gene from transposon Tn5. Gene. 1982; 19: 327-336.
8. Adra, C N, Boer, P H, McBurney, M W. Cloning and expression of the mouse pgk-1 gene and the nucleotide sequence of its promoter. Gene. 1987; 60: 65-74.
9. Yagi, T, Ikawa, Y, Yoshida, K, et al. Homologous recombination at c-fyn locus of mouse embryonic stem cells with use of diphtheria toxin A-fragment gene in negative selection. Proc Natl Acad Sci USA. 1990; 87: 9918-9922.
10. Valenzuela, D M, Murphy, A J, Frendewey, D, et al. High-throughput engineering of the mouse genome coupled with high-resolution expression analysis. Nat. Biotechnol. 2003; 21: 652-659.
11. Indra, A K, Warot, X, Broard, J, et al. Temporally-controlled site-specific mutagenesis in the basal layer of the epidermis: comparison of the recombinase activity of the tramoxifen-inducible Cre-ER(T) and Cre-ER(T2) recombinases. Nucleic Acids Res. 1999; 27: 4324-4327.
12. Clemens, T L, Tang, H, Maeda, S, et al. Analysis of osteocalcin expression in transgenic mice reveals a species difference in vitamin D regulation of mouse and human osteocalcin genes. J Bone Miner Res. 1997; 12: 1570-1576.
13. Yoshikawa, Y, Kode, A, Xu, L, Mosialou, I, Silva B C, Ferron M, Clemens T L, Economides A N, Kousteni, S. Genetic Evidence Points to an Osteocalcin-Independent Influence of Osteoblasts on Energy Metabolism. J Bone and Miner Res. 2011; 26, No. 9, 2012-2025.

What is claimed is:

1. A method comprising, identifying a subject having or at risk of developing a disorder selected from the group consisting of Type 1 diabetes, Type 2 diabetes, metabolic syndrome, obesity or obesity-related disease, and administering to the subject a therapeutically effective amount of Lcn-2 or a biologically active fragment or variant for treating or preventing the disorder.

2. The method of claim 1, wherein Lcn-2 or a biologically active fragment or variant is administered to the subject in an amount effective to produce an effect selected from the group consisting of a significant increase in pancreatic beta-cell proliferation, insulin expression, insulin sensitivity, glucose tolerance, bone mass, and serum adiponectin levels, and a significant decrease in serum resistin levels, weight gain, fat mass, and weight loss.

3. The method of claim 1, wherein the subject is a human.

4. The method of claim 1, wherein Lcn-2 or a biologically active fragment or variant thereof is administered in combination with another agent known to treat the disorder.

5. The method of claim 1, wherein the therapeutically effective amount of Lcn-2 or a biologically active fragment or variant is from about 4 micrograms/kg to about 60 micrograms/kg of the subject's body weight.

6. A method comprising, administering Lcn-2 or a biologically active fragment or variant to a subject, in an amount that causes an effect selected from the group consisting of significantly increasing pancreatic beta-cell proliferation, insulin expression, insulin secretion, insulin sensitivity, weight loss, bone mass, serum adiponectin levels and glucose tolerance, and significantly decreasing weight gain, fat mass, and serum resistin levels in the subject.

7. The method of claim 6, wherein the subject is human.

8. A pharmaceutical composition comprising a therapeutically effective amount of Lcn-2 or a biologically active fragment or variant to treat or prevent a disorder in a subject selected from the group consisting of diabetes, metabolic syndrome, and obesity or obesity-related disease in a subject, which amount produces an effect selected from the group consisting of a significant increase in pancreatic beta-cell proliferation, insulin expression, insulin sensitivity, weight loss, bone mass, serum adiponectin levels, and glucose tolerance, and a significant decrease in weight gain, fat mass, and serum resistin levels in the subject.

9. The pharmaceutical composition of claim 8 further comprising a therapeutically effective amount of an agent selected from the group consisting of resistin, adiponectin, and therapeutic oligonucleotides that reduce the expression or biological activity of FoxO1.

10. A kit comprising the pharmaceutical composition of claim 9.

11. A method comprising identifying a subject having or at risk of developing Type 1 diabetes, Type 2 diabetes, metabolic syndrome, and obesity or obesity-related disease, and administering a therapeutically effective amount of an agent that reduces FoxO1 expression or FoxO1 activity.

12. The method of claim 11, further comprising administering a therapeutically effective amount of Lcn-2.

13. The method of claim 11, wherein the method reduces FoxO1 expression or FoxO1 activity in osteoblasts.

14. The method of claim 11, wherein the therapeutically effective amount of the agent significantly increases serum Lcn-2 levels.

15. The method of claim 11, wherein the agent is selected from the group consisting of antisense DNA or RNA, micro RNAs, and microRNA mimics, short hairpin RNA(shRNAs), ribozymes, and small interfering RNA (siRNAs).

16. The method of claim 15 wherein said micro RNA is selected from the group consisting of miR-182, miR-96, miR-183 and miR-135b.

17. A method comprising, identifying a subject having or at risk of developing a bone disorder, and administering to the subject a therapeutically effective amount of Lcn-2 or a biologically active fragment or variant thereby for treating or preventing the disorder.

18. The method of claim 17, wherein the disorder of the bone disorder is selected from the group consisting of osteoporosis, osteopenia, osteomyelitis, and osteoarthritis.

19. The method of claim 18, wherein the subject is a human.

20. The method of claim 18, further comprising administering a therapeutically effective amount of an agent that reduces FoxO1 expression or FoxO1 activity.

21. A method comprising identifying a subject having or at risk of developing a muscle disorder, and administering a therapeutically effective amount of Lcn-2 or a biologically active fragment or variant to the subject in an amount that increases or maintains myogenesis thereby treating or preventing the muscle disorder.

22. The method of claim 21, wherein the subject is a human.

23. The method of claim 21, wherein the disorder is selected from the group consisting of muscle atrophy, muscular dystrophy, fibromyalgia, myositis, polymyositis, myopathy, rhabdomyolysis, inflammatory muscle disease, MCAD and other fatty acid oxidation disorders and carnitine/acylcarnitine translocase deficiency (CACT).

24. The method of claim 21, further comprising administering a therapeutically effective amount of an agent that reduces FoxO1 expression or FoxO1 activity.

25. A method comprising contacting pancreatic beta cells in vivo or in vitro with Lcn-2 or a biologically active fragment or variant in an amount that increases beta-cell area or beta-cell numbers.

26. The method of claim 25, wherein the beta cells are human cells.
